(19) United States
(12) Patent Application Publication
(10) Pub. No.: US 2013/0190324 A1
(43) Pub. Date: Jul. 25, 2013

(54) TOPICAL OCULAR DRUG DELIVERY

(71) Applicant: The Regents of the University of Colorado, a body corporate, Denver, CO (US)

(72) Inventors: Uday Kompella, Englewood, CO (US); Sunil Vooturi, Denver, CO (US); Rajendra Kadam, Aurora, CO (US)

(73) Assignee: The Regents of the University of Colorado, a body corporate, Denver, CO (US)

(21) Appl. No.: 13/726,013

(22) Filed: Dec. 22, 2012

Related U.S. Application Data
(60) Provisional application No. 61/580,071, filed on Dec. 23, 2011.

(51) Int. Cl. A61K 47/48

(52) U.S. Cl. CPC A61K 47/48038
USPC 514/253.08; 544/363; 548/377.1; 514/406

(57) ABSTRACT

The present invention provides compositions and methods for increasing the delivery (i.e., bioavailability) of a compound to an ocular cell. Such compositions and methods can be used to treat an ocular clinical condition. Typically, increased bioavailability or delivery of the compound to ocular cells is achieved by utilizing a membrane transporter.

### CROSS-REFERENCE TO RELATED APPLICATIONS

This application claims the priority benefit of U.S. Provisional Application No. 61/580,071, filed Dec. 23, 2011, which is incorporated herein by reference in its entirety.

### STATEMENT REGARDING FEDERALLY FUNDED RESEARCH

This invention was made with government support under grant numbers EY018940 and EY017533 awarded by the National Institutes of Health. The government has certain rights in the invention.

### FIELD OF THE INVENTION

The present invention relates to topical ocular drug delivery compositions and methods for using the same. In particular, the present invention relates to compositions and methods that utilize membrane transporters to increase the amount of compound delivered to ocular cells.

### BACKGROUND OF THE INVENTION

Intraocular drug delivery is major challenge because of unique barrier properties offered by nature to eye as protective mechanism. The most commonly used topical route typically results in less than 5% bioavailability in the anterior segment eye tissue and less than 0.05% in the posterior segment eye tissues due in part to rapid clearance of drug from the ocular surfaces by blinking and tear drainage, and poor permeability across the cornea and conjunctiva. Topically applied drug molecules have access to the intraocular tissues by permeability across ocular barriers either by transporter mediated active transport or passive diffusion which include both paracellular and transcellular routes. Passive permeability of drugs across the cornea and conjunctiva is limited by inter alia very tight epithelial junctions as well as the multilayers of the corneal and conjunctival epithelium.

Due to poor permeability of drug molecules across the ocular barriers and rapid clearance from the ocular surface, frequent multiple eye drops are needed to maintain the therapeutically effective concentration in the target anterior segment tissues. Regardless of frequency and dosage of application, in general topical delivery of drug molecules to the posterior segment ocular tissues including retina and vitreous humor is negligible. Unfortunately, systemic drug delivery to the retina is also limited due to blood retinal barriers, which include RPE (outer) and retinal endothelial cells (inner).

Topically or systemically administered drug must be absorbed through ocular barriers to reach target tissues. Some drugs get absorbed through biological barriers by passive diffusion to some extent and by active carrier mediated transport. Various formulation approaches have been contemplated to enhance the passive diffusion of drug molecules across biological barriers. One of the most commonly used methods is to use permeability enhancer, which compromises the integrity of barriers and enhances the diffusion of drug; however, such a method often has toxic effects as it also results in enhanced permeability to other molecules and antigens.

Accordingly, there is a continuing need for enhanced drug delivery to ocular tissues.

### SUMMARY OF THE INVENTION

Some aspects of the invention provide methods and compositions for drug delivery to the intraocular tissues after topical application. In particular, some aspects of the invention is based on the discovery by the present inventors that transporter mediated delivery of ion-drug pair complex can be used effectively for delivery of the drug to ocular tissues.

In some embodiments, poor permeability of drug across ocular barriers can be overcome by utilizing transporter mediated delivery of drug ion pair complex to topically administer drugs to ocular tissues. In one particular embodiment, a commonly used ocular drug was delivered effectively across ocular barriers using a drug-ion pair complex with amino acids such as, L-arginine (ARG) and L-lysine (LYS) as counterions. Use of such counterions increased the transporter mediated delivery of the drug. As used herein, any rate and/or the level of drug delivery increase in drug-ion pair complex using a particular counterion are relative to the corresponding rate and/or the level of drug delivery without formation of a drug-ion complex and/or use of the counterions disclosed herein.

In some embodiments, methods and compositions of the invention increase delivery of the topically applied ocular drug across the ocular barriers by at least 150%, typically at least 180%, and often at least 220%.

### BRIEF DESCRIPTION OF THE DRAWINGS

FIG. 1 is graphs of comparison of cumulative % transport as function of time of GFX and GFX-ARG drug-ion pair complex across albino rabbit A) cornea and B) SCRPE. Data is expressed as mean±SD for n=4. GFX-ARG ion pair showed enhanced permeability than GFX across rabbit cornea and SCRPE.

FIG. 2 is graphs of comparison of cumulative % transport as a function of time for GFX and GFX-LYS across albino rabbit A) cornea and B) SCRPE. Data is expressed as mean±SD for n=4. GFX-LYS ion pair showed no significant enhancement in permeability than GFX across rabbit cornea and SCRPE.

FIG. 3 is graphs showing the effect of the counterion ARG concentration in GFX-ARG on transport of GFX across albino rabbit A) cornea and B) SCRPE. Data is expressed as mean±SD for n=4. Increasing the counterion ARG concentrations from 1:1 ratio to 1:3 ratio of GFX to ARG in the GFX-ARG ion pair resulted in decreased transport across both cornea and SCRPE, possibly due to competition by the excess ARG for delivery via membrane transporter.

FIG. 4 is graphs showing in vitro cumulative % transport of GFX-ARG in the presence and absence of ATB⁰⁺ inhibitor across albino rabbit A) cornea and B) SCRPE, in the presence and absence of OCT inhibitor across C) cornea and D) SCRPE and in presence and absence of the OCTN inhibitor across E) cornea and F) SCRPE. Data is expressed as mean±SD for n=4. Graphs indicate that active transport of GFX-ARG across rabbit cornea and SCRPE is mediated by organic cation transporters (OCT).

FIG. 5 is graphs showing ocular tissue distribution of GFX at the end of 1 hr following single topical application of the GFX-ARG ion pair and GFX solution in PBS. Data is expressed as mean±SD for n=4. *p<0.05 when compared with GFX. Inserts are magnified version for retina and vitreous. GFX-ARG showed enhanced ocular delivery to all ocular tissues compared to GFX solution after single topical application as an eye drop (30 μl) in pigmented rabbits.

FIG. 6 is a graph of the ratio of drug delivery at 1 hr following single topical dosing with 30 μl of GFX-ARG or GFX in pigmented rabbits. Data are expressed as mean for n=4. Black thick horizontal line at 1 is the reference line for equal delivery between GFX-ARG and GFX groups.

FIG. 7 is a graph showing effects of directionality and transporter specific inhibitors on transport of A) Gly-Sar, B) MPP⁺, C) L-tryptophan, and D) Phenyl acetic acid across human sclera-choroid-RPE. Data are expressed as mean±sd for n=4. Data show that sclera to retina direction transport was significantly higher than retina to sclera transport for gly-sar, MPP+ and L-tryptophan. For phenyl acetic acid, data show that retina to sclera transport was higher. For the first time, this data shows that in human sclera-choroid-RPE, PEPT, OCT, and ATB^{0,+} transporters transport drugs from outside (tear-side) to inside (vitreous-side), suggesting their suitability for inward transport in human eyes. MCT, on the other hand, transports drugs from inside to the outside across human sclera-choroid-RPE. Data show that sclera to retina direction transport of gly-sar, MPP⁺ and L-tryptophan was significantly inhibited in the presence of transporter specific inhibitors. This study confirms the activity of PEPT, OCT, and ATB^{0,+} transporters in human sclera-choroid-RPE. Further, histological studies demonstrated the expression of PEPT-1, PEPT-2, OCT-1, and MCT-3 proteins in the retinal pigment epithelium. Histology also showed evidence for the presence of ATB^{0,+}, PEPT, and MCT in the neural retina.

FIG. 8 is a graph showing effects of an inhibitor on transport of A) Gly-Sar, B) MPP⁺, C) L-tryptophan, and D) Phenyl acetic acid across human cornea. Data are expressed as mean±sd for n=4. Data show that transport of gly-sar, MPP⁺, L-tryptophan, and phenyl acetic acid was significantly inhibited in the presence of an inhibitor. This data indicates that PEPT, OCT, ATB^{0,+} and MCT are active in human cornea in the outside (tear-side) to inside (aqueous humor) direction.

FIG. 9 is a graph showing that cumulative % transport of GFX-OCT prodrug was significantly (p<0.01) higher than GFX across A) cornea, B) conjunctiva, and C) SCRPE. Cornea and SCRPE were from NZW rabbit and conjunctiva was from bovine eyes. GFX-OCT transport was significantly inhibited by MPP+(competitive inhibitor of OCT) across all tissues (p<0.005). Data were expressed as mean±SD for n=4.

FIG. 10 is a graph showing that cumulative % transport of GFX-MCT prodrug was significantly (p<0.01) higher than GFX across A) cornea, B) conjunctiva, and C) SCRPE. Cornea and SCRPE were from NZW rabbit and conjunctiva was from bovine eyes. GFX-MCT transport was significantly inhibited by nicotinic acid (competitive inhibitor of MCT) across conjunctiva. Data were expressed as mean±SD for n=4.

FIG. 11 is a graph showing ocular distribution of GFX and GFX-OCT prodrug at 1 h after their topical eye drop application in pigmented rabbits. Levels of prodrug represent the sum of the GFX formed and unchanged prodrug. GFX-OCT prodrug levels were higher in vitreous (3.6-fold) and CRPE (1.95-fold) compared to GFX (p<0.05). Data are expressed as mean±SD for n=4 animals.

FIG. 12 shows a graph of fold change in ATP-binding cassette (ABC) transporters expression in hypoxic rat choroid-retina when relative to normoxic rat choroid-retina. Values above +1 indicate the up regulation and values below −1 indicate the down regulation of transporters in hypoxic condition. Thick black lines at ±1.5 are cutoff lines for 50% up regulation and down regulation. Data is expressed as mean for three biological replicates.

FIG. 13 shows a graph of fold change in solute carrier transporters (SLC) expression in hypoxic rat choroid-retina relative to normoxic rat choroid-retina. Values above +1 indicate the up regulation and values below −1 indicate the down regulation of transporters in hypoxic condition. MCT-3, GLUT-B, Cystine/Glutamate transporter, CAT4, ENT1, and ENT2 were significantly upregulated in hypoxic rat choroid-retina, suggesting their potential use for enhanced drug delivery. Thick black lines at ±1.5 are cutoff lines for 50% up regulation and down regulation. Data is expressed as mean for three biological replicates.

FIG. 14 shows a graph of fold change in miscellaneous transporter expression in hypoxic rat choroid-retina relative to normoxic rat choroid-retina. Values above +1 indicate the up regulation and values below −1 indicate the down regulation of transporters in hypoxic condition. Thick black lines at ±1.5 are cutoff lines for 50% up regulation and down regulation. Data is expressed as mean for three biological replicates.

FIG. 15 is graphs showing that transport of Gly-Sar, MPP+, and valacylovir is significantly higher across normoxic calf SCRPE than hypoxic calf SCRPE. On the other hand, transport of phenylacetic acid is significantly higher across hypoxic SCRPE than normoxic SCRPE. Transport of all four transporter substrates was significantly inhibited in the presence of inhibitor cocktail. A) Gly-Sar; B) MPP+; C) Valacylovir; and D) Phenylacetic acid. Data are expressed as mean±SD for n=4.

FIG. 16 is graphs showing apparent permeability (Papp) of Gly-Sar, MPP+, and valacylovir is significantly higher across normoxic SCRPE than hypoxic SCRPE. For phenylacetic acid, Papp is significantly higher across hypoxic SCRPE than normoxic SCRPE. Apparent permeability of all four transporter substrates was significantly inhibited in the presence of inhibitor cocktail. Effect of hypoxia and transporter inhibitors on apparent permeability of A) Gly-Sar, B) MPP+, C) Valacylovir, and D) Phenylacetic acid across normoxic and hypoxic calf SCRPE. Data are expressed as mean±SD for n=4. * Significantly different from normoxic at P≦0.05. + Significantly different from hypoxic at P≦0.05

FIG. 17 is graphs showing transport of Gly-Sar, MPP+, and valacylovir is significantly higher across normoxic calf cornea than hypoxic calf cornea. For phenylacetic acid, transport across hypoxic cornea is significantly higher than normoxic cornea. A) Gly-Sar; B) MPP+; C) Valacylovir; and D) Phenylacetic acid. Data are expressed as mean±SD for n=4.

FIG. 18 is graphs showing apparent permeability (Papp) of Gly-Sar, MPP+, and valacylovir is significantly higher across normoxic cornea than hypoxic cornea> For phenylacetic acid, Papp is significantly higher across hypoxic cornea than normoxic cornea. Effect of hypoxia on apparent permeability of A) Gly-Sar, B) MPP+, C) Valacylovir, and D) Phenylacetic acid across calf cornea. Data are expressed as mean±SD for n=4. *Significantly different from normoxic at P≦0.05.

FIG. 19 is graphs showing relative gene expression of PEPT, ATB⁰⁺, OCT, and MCT transporters in hypoxic rat choroid-retina, normalized to normoxic rat choroid-retina. Data are expressed as mean for n=3. Gene expression in normoxic animal was set to 100% and relative change in hypoxic animal was expressed in % up regulation

### DETAILED DESCRIPTION OF THE INVENTION

Drug delivery to the intraocular tissue is hindered by barriers present in the eye. Some aspects of the invention provide methods and compositions to overcome this problem of delivering a therapeutically effective amount of a drug to treat various ocular ailments (i.e., clinical ocular conditions). The present invention is based in part on the discovery by the present inventors of the expression of various solute carrier transporters in human ocular tissues including cornea, conjunctiva, ciliary epithelium, and choroid-retina by immunohistochemistry. The invention is also based in part on the discovery by the present inventors of the activity of transporters of specific substrates across isolated human sclera-choroid-RPE and cornea in the presence and the absence of a corresponding transporter inhibitor. Furthermore, some aspects of the invention are based on the discovery by the present inventors of the directionality of transporters. Additionally, some aspects of the invention are based on the discovery by the present inventors of the up regulation or down regulation of transporters under oxidative stress.

The present inventors have discovered that membrane transporters, such as PEPT-1, PEPT-2, ATB⁰⁺, OCT-1, OCT-2, MCT-1 and MCT-3, are expressed in human ocular tissues. For example, it was found that PEPT-2 showed an abundance of expression in cornea epithelium, conjunctival epithelium, retinal pigmented epithelium (RPE), and outer segment of photoreceptor cells relative to other transporters. PEPT-1 showed the expression in all tissues studied with a relatively higher abundance in ciliary epithelium and outer plexiform layer of retina. Out of two isoforms of organic cation transporter (OCT), OCT-1 showed expression in all ocular tissues, whereas OCT-2 expression appeared to be limited to corneal and conjunctival epithelium. Amino acid transporter (ATB⁰⁺) showed expression in cornea epithelium, conjunctival epithelium, retinal pigmented epithelium (RPE), as well as neural retina. MCT-1 showed expression in all tissues, whereas the expression of MCT-3 appeared to be localized to RPE layer.

In vitro transport study showed the transporter mediated inward transport of Gly-Sar (PEPT substrate), MPP⁺ (OCT substrate), and L-tryptophan (ATB⁰⁺) across cornea as well as SCRPE. Inward transport of Gly-Sar, MPP⁺, and L-tryptophan was significantly inhibited in the presence of a corresponding transporter inhibitor. For phenyl acetic acid (MCT substrate), retina to sclera transport was significantly higher than inward transport. This result indicates MCTs are acting as efflux transporters. For cornea, inward transport of phenyl acetic acid transport was significantly higher than outward transport. Inward transport of phenyl acetic acid was inhibited in the presence on MCT inhibitors.

The present inventors have discovered that peptide transporter (PEPT), OCT and ATB⁰⁺ are influx transporter present in human ocular barriers limiting drug delivery to retina. Accordingly, some aspects of the invention provide methods and compositions utilizing these findings in a transporter guided retinal drug delivery. Compositions of the invention can be administered topically, transsclerally, or suprachoroidally. Additionally since transporters were also identified in the neural retina, transporter guided drugs can also be administered subretinally or intravitreally to enhance delivery to retinal cells. Other local ocular routes and/or systemic delivery can also be employed in conjunction with transporter guided drug compositions to treat various ocular ailments. However, typically methods and compositions of the invention are administered topically.

A safer approach of drug delivery is to use body's own biological mechanisms such as plasma membrane transporter in delivery of drug to the target site. Mammalian cells express various transporters on plasma membrane to aid in delivery of essential nutrients to cells from extracellular matrix. These transporters can be utilized for delivery of drug molecules into cell that has structural resemblance to the transporter substrate.

There are various examples in literature showing the transport of small drug molecule across plasma membrane by solute carrier transporters. In addition, various attempts have been made to enhance the delivery of poorly permeable drugs by making them a substrate for particular transporter using prodrug approach. One of the examples for transporter guided prodrug delivery is valacylovir, an L-valine ester of acyclovir, that results in three to five fold increase in oral bioavailability as compared to parent acyclovir. Transport of valacylovir across human intestinal epithelium is believed to be mediated by oligopeptide and amino acid transporters. Valacyclovir is a compound that has a covalent linkage between L-valine and acyclovir. Thus, in order to use such a “prodrug” delivery system, one requires actual synthesis of a covalently linked drug and a delivery system. Such an approach is feasible based on the current invention. Such synthesis adds additional costs and time for each desired drug modification, and therefore, is not universally applicable. This invention has an additional approach as well for transporter guided delivery.

While transporter guided drug delivery has been studied for oral delivery of drugs for central nervous system (CNS) and other ailments, currently there is a very little, if any, studies or reports in transport guided ocular drug delivery. In addition, to date, no report is available in the literature showing the functional characterization of drug transporters in human ocular tissues.

One can in theory improve the ocular bioavailability of topically applied drug molecules using various formulation approaches. Commonly used approaches include the use of a permeability enhancer that compromises the barrier integrity and enhances drug diffusion. Another method is to use a viscosity enhancer that increases the precorneal retention by reducing the tear drainage. Other attempts have been to use a prodrug approach to chemically modify the active drug molecules to change their physicochemical properties. Modification of physicochemical properties such as lipophilicity, solubility, or pigment binding using the prodrug approach showed significant improvement in ocular delivery of some drugs.

To date there are no reports available which have showed the utilization of drug transporters for drug-ion pair delivery.

Some aspects of the invention are based on the discovery of the transporter mediated enhanced delivery or permeability of ion pair complexes across ocular barriers and characterization or identification of such transporters by the present inventors. The amphiphillic, ophthalmic antibiotic gatifloxacin (GFX) is known to be poorly permeable. GFX is a fourth generation fluoroquinolone antibiotic approved for bacterial conjunctivitis and is commonly used as an off label treatment of vitreal endophthalmitis. However, as stated above, it is well known that GFX suffers from poor permeability across the ocular barriers.

Initially, the present inventors believed that the cationic amino acids arginine (ARG) and lysine (LYS) as counterions of GFX may serve as substrates for the amino acid transporters (ABT⁰⁺). Moreover, the presence of guanidine group in ARG could also serve as a substrate for the organic cation transporter (OCT). Accordingly, the permeability across the isolated rabbit cornea and SCRPE were determined. Transporter mediated transport was evaluated by permeability studies in the presence and absence of specific inhibitors. In vivo ocular delivery was evaluated in rabbits after single topical application of the GFX-ARG ion-pair complex. Computational modeling was also performed to predict GFX-ARG interactions with rabbit OCTs. It should be appreciated that the term “ion-pair” as used herein refers to the presence of an ionic bond between a compound or a drug and the counterion such as ARG, LYS, or other suitable counterion readily known to one skilled in the art having read the present disclosure.

While many ion-pair complexes are highly soluble in water (e.g., sodium chloride and other metallic salts), the aqueous solubility of ion-pair complexes of the invention are about 9.5 mg/mL or less, typically about 11.5 mg/mL or less, and often about 15.0 mg/mL or less. Thus, ion-complexes of the invention generally form a tight ion-pair complex in vitro and in vivo.

Various aspects of the invention provide methods and compositions for enhancing solubility and/or delivery of a compound or a drug (e.g., therapeutically active agent) for treating a clinical ocular condition. As used herein, the terms “clinical ocular condition” and “ocular condition” and “ocular clinical condition” are used interchangeably herein and refer to a non-normal clinical condition of ocular tissue(s) or eye. Exemplary clinical ocular conditions include, but are not limited to, inflammation, infection (e.g., bacterial and/or viral infection), allergy, dry eye, glaucoma, tissues before and after surgery, diabetic retinopathy, retinal degenerative diseases, macular degeneration, vascular occlusions, optic neuropathy, cataracts, posterior capsular opacification, corneal angiogenesis, other neovascular diseases of the eye, thyroid eye disease, retinoblastoma, uveal melanoma, and endophthalmitis.

In some aspects of the invention, a prodrug is used to increase the solubility or drug delivery across ocular barriers by a membrane transporter. As used herein, the term “prodrug” refers to a pharmacologically less active derivative of a parent drug molecule that requires biotransformation, either spontaneous or enzymatic, within the organism to release the active drug. Prodrugs are variations or derivatives of pharmaceutically active compounds that have groups cleavable under metabolic or in vivo conditions. Prodrugs become the pharmaceutically active compounds in vivo, for example, when they undergo solvolysis under physiological conditions or undergo enzymatic degradation. Prodrugs can undergo a number of biotransformation steps required to release the active drug within the desired ocular tissue. Prodrug forms often offer advantages of solubility, enhanced delivery, tissue compatibility, and/or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include acid derivatives that are well known to one skilled in the art, such as, but not limited to, esters prepared by reaction of the parent acids with a suitable alcohol, or amides prepared by reaction of the parent acid compound with an amine, or basic groups reacted to form an acylated base derivative. Moreover, the prodrug of this invention may be combined with other features herein taught to enhance bioavailability, solubility, and/or other desired physical properties.

One particular aspect of the invention provides a composition comprising an ocular drug-ion pair complex. As used herein, the term “ocular drug” refers to any compound or molecule that can be used to treat a clinical ocular condition and includes its salt, prodrug, and a derivative thereof. Compositions of the invention are useful in treating a clinical ocular condition. In some embodiments, the prodrug increases solubility or drug delivery across ocular barriers by a membrane transporter. In some embodiments, the solubility or drug delivery of the prodrug is at least 125%, typically at least 150%, often at least 175%, and more often at least 200% that of the prodrug's parent compound.

Generally, the membrane transporter that is utilized in membrane transporter mediated delivery of the ocular drug-ion pair complex is selected from the group consisting of or comprises an organic cation transporter (OCT), a monocarboxylate transporter (MCT), an amino acid transporter (ATB^{0,+}), a peptide transporter (PEPT), and/or a combination thereof. Exemplary membrane transporters that can be utilized to increase the bioavailability include, but are not limited to, OCT-1, OCT-2, MCT-1, MCT-3, PEPT-1, PEPT-2, ATB⁰⁺, OCTN1, OCTN2, and TAUT.

In some embodiments, by using the ocular drug-ion pair complex, the amount of ocular drug delivery to a desired ocular cells or tissue can be increase by at least 200%, typically at least 300%, and often at least 350% compared to using ocular drug under the same or similar condition in the absence of the counterion. Yet in other embodiments, by using the ocular drug-ion pair complex, the kinetic rate of ocular drug delivery is increase by at least about 150%, typically at least about 200% and often at least about 250%.

In some embodiments, the ocular drug or a therapeutically active agent comprises fluoroquinolones, analogs of prostaglandins, beta-blockers, non-steroidal anti-inflammatory drugs, corticosteroids, anti-angiogenic agents, neuroprotective agents, cell survival agents, anti-proliferative agents, and apoptotic agents, or a combination thereof. Some specific examples of ocular drugs include, but are not limited to, gatifloxacin, besifloxacin, pazopanib, budesonide, celecoxib, diclofenac, ketorolac, nepafenac, bromfenac, nimesulide, timolol, brimonidine, and betaxolol.

Still in other embodiments, the ocular drug is an anti-inflammatory ocular drug, an anti-infective ocular drug, anti-allergy drug, intra ocular pressure lowering drug, anti-angiogenic drug, vascular stabilizing agent, cytoprotective or neuroprotective agent, anti-tumor agent, anti-proliferative agent, or a combination thereof.

Other aspects of the invention provide methods for treating an ocular condition in a subject by administering to a subject in need of such a treatment a therapeutically effective amount of a composition disclosed herein.

In some embodiments, the ocular condition comprises inflammation, microbial infection, allergy, dry eye, glaucoma, surgery, diabetic retinopathy, retinal degeneration, macular degeneration, vascular occlusions, optic neuropathy, cataracts, posterior capsular opacification, corneal angiogenesis, other neovascular diseases, thyroid eye disease, retinoblastoma, uveal melanoma, endophthalmitis, or a combination thereof.

Yet other aspects of the invention provide methods for increasing the solubility or delivery of a therapeutically active compound for treating a clinical ocular condition. In some embodiments, such methods comprise admixing a therapeutically active compound with arginine or other suitable counterion to form a compound-ion pair complex. Such embodiments can increase the solubility and/or the bioavailability by at least 125%, typically at least 150%, and often at least 175% compared to the same compound in the absence of the counterion.

Table below summarizes some of the transporters that were found by the present inventors at various ocular tissues.

Summary of immunohistochemical localization of drug
transporters in ocular tissues of human eye.

| Ocular Tissue | PEPT-1 | PEPT-2 | OCT-1 | OCT-2 | MCT-1 | MCT-3 | ATB⁰⁺ |
|---|---|---|---|---|---|---|---|
| Cornea | + | + | + | + | + | − | + |
| Conjunctiva | + | + | + | + | + | − | + |
| Ciliary Epithelium | + | + | + | − | + | − | + |
| Choroidal smooth muscle | + | + | + | − | − | − | − |
| Retinal pigmented | + | + | + | − | − | + | − |
| epithelium (RPE) | | | | | | | |
| Outer segment of | − | + | − | − | + | − | − |
| photoreceptor cells | | | | | | | |
| Inner segment of | − | + | + | − | − | − | − |
| Photoreceptor cells | | | | | | | |
| Outer nuclear layer | − | − | − | − | − | − | + |
| Inner nuclear layer | − | − | − | − | − | − | + |
| Outer plexiform layer | + | − | − | − | − | − | − |
| Inner plexiform layer | − | − | − | − | − | − | − |
| Ganglion cell layer | + | + | − | − | − | − | + |
| Inner limiting membrane | − | − | − | − | + | − | − |

(+) indicate the presence of transporter and (−) absence of transporter.

Exemplary counterions that are useful in compositions and methods of the invention include, but are not limited to, arginine, arginine oligomers (e.g., having arginine monomeric units of from about 2 to 6), other small molecules comprising a guanidine group or aliphatic or alicylic tertiary amine group, primary amine group, secondary amine group or pyridine group or histidine group or amino acids or aliphatic and alicyclic carboxylic acid group.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Materials:

MPP⁺ iodide (≧98.0%), α-methyl-DL-tryptophan (98%), L-arginine, L-lysine, phenyl acetic acid (98%), L-tryptophan (>98%), Gly-Sar, metformin (˜97%), nicotinic acid sodium salt, nadolol (˜98%) and formic acid were purchased from Sigma-Aldrich (St. Louis, Mo.). H-Pro-Phe-OH (>99%) was purchased from Bachem (Torrance, Calif.). Gatifloxacin (GFX) was purchased from Enzo Life Sciences Inc. (Farmingdale, N.Y.). Moxifloxacin was purchased from Selleck Chemicals LLC (Houston, Tex.). HPLC grade acetonitrile and methanol were purchased from Fisher Scientific (Fair Lawn, N.J.). Ammonium formate (99.9%) was purchased from Fluka BioChemika (USA). All primary antibodies except ATB⁰⁺ antibody were purchased from Santa Cruz Biotech, (Santa Cruz, Calif.). ATB⁰⁺ antibody was purchased from Medical and Biological Laboratories, Japan. All other chemicals and reagents used in this study were of analytical reagent grade.

### Example 1

Determination of Aqueous Solubility of GFX and Ion Pair Complex:

The aqueous solubility of GFX, GFX-ARG, and GFX-LYS was determined in phosphate buffer saline (PBS) pH 7.4 at 37° C. Solubility was measured by adding an excess amount of GFX (10 mg) to 0.5 ml of PBS containing 1.0 and 3.0 mole equivalent amount of ARG or LYS. PBS without amino acid was included as control. Samples were incubated at 37° C. for 24 hr in incubator shaker with constant shaking at 200 rpm. At the end of 24 hr of incubations, samples were filtered through 0.45 μm filter and filtrates were analyzed for drug content. All experiments were performed in triplicate.

In Vitro Transport Across Albino Rabbit Cornea and Sclera-Choroid-RPE:

In vitro transport of GFX, GFX-ARG, and GFX-LYS were carried out across the New Zealand white rabbit cornea and sclera-choroid-RPE (SCRPE). Rabbit eyes were obtained within 24 hr of harvesting from Pel-Freez Biologicals (Rogers, Ark.) and shipped overnight in Hank's balanced salt solution on wet ice. Eyes were used immediately upon arrival. Eyes were washed with assay buffer and cleaned from the muscle and unwanted tissues. For isolation of the cornea, an incision was made 2 mm posterior to the cornea-sclera junction circumferentially around the globe. The small part of the sclera left with cornea helps in mounting the cornea on Ussing chambers. For isolation of the SCRPE, anterior and posterior parts were separated by a circumferential cut at the limbus. The vitreous was squeezed out and the neural retina was separated from the choroid-RPE by filling the eye cup with assay buffer, and the floating retina was collected. After separation of the retina, the eye cup was flattened with small cuts around the globe. Isolated tissues were mounted on modified Ussing chambers (Navicyte, Sparks, Nev.) such that the episcleral side of SCRPE or the epithelial side of the cornea is facing to the donor chamber. Chambers were filled with 1.5 ml of assay buffer with (donor side) or without (receiver side) drug (100 μM). A circulating water bath was used to maintain the temperature at 37° C. during the transport study. The pH of the assay buffer was maintained at 7.4 using 95% air-5% CO₂ aeration which also helps in stirring of bathing solutions. Samples were collected (200 μL) from the receiver side every 30 minute for 3 hr and the lost volume was replaced with fresh assay buffer pre-equilibrated at 37° C. GFX levels were analyzed using an LC-MS/MS assay. Permeation data were corrected for dilution of the receiver solution with sample volume replenishment.

Elucidation of Transport Mechanism:

To elucidate the mechanism for enhanced permeability of GFX-ARG (1:1) ion pair, transport studies were carried out in the presence and absence of specific transporter inhibitors. Since amino acid transporters (ATB⁰⁺) are present in the human and rabbit cornea and SCRPE, while the counterion ARG is a substrate for ATB⁰⁺, the present inventors theorized that the GFX-ARG could be transported by the ATB⁰⁺ transporter. Transport studies were therefore carried out in the presence of the ATB⁰⁺ inhibitor α-methyl-DL-tryptophan (500 μM). Further, ARG contains a guanidine group, and GFX-ARG can transported by the organic cation transporters (OCT), so transport studies were also carried out in the presence and absence of the OCT transporter competitive inhibitor MPP⁺ (500 μM) and the carnitine/organic cation transport (OCTN) competitive inhibitor L-carnitine (500 μM).

In Vivo Tissue Distribution Study in Rabbits:

Male New Zealand Satin rabbits in the weight range of 1.8 to 3 kg were obtained from Western Oregon Rabbit Company (Philmoth, Oreg.). Rabbits were divided into two groups (2 animals each), one group received GFX solution (5 mg/ml) in sterile phosphate buffer saline (PBS), and the other group received the GFX-ARG (1:1) ion pair complex (5.0 mg/ml) solution in PBS. Rabbits were restrained in a rabbit restrainer and allowed to stabilize for 5-10 minutes. A topical eye drop (30 μl) of drug solution was applied in both eyes of rabbits using a positive displacement pipette (Gilson 10-100 μl) and sterile tips. To minimize the runoff of instilled dose, the eyelids were gently closed for few seconds after dosing. The time of dose administered was recorded for each animal. After 1 hr of dosing, blood samples were collected from the marginal ear vein and rabbits were euthanized by intravenous injection of sodium pentobarbitone (150 mg/kg) in the marginal ear vein. Eyes were then enucleated immediately after euthanasia using surgical accessories and snap frozen immediately in dry ice: isopentane bath and stored at −80° C. until dissection. Eyes were dissected in the frozen condition using dry ice: isopentane bath and ceramic tile to avoid thawing of the eye during dissection. Various ocular tissues including cornea, conjunctiva, aqueous humor, iris-ciliary body, sclera, choroid-RPE, retina, lens, and vitreous humor were collected and transferred into labeled tubes and stored at −80° C. until further processing.

Tissue Sample Processing for LC-MS/MS Analysis:

GFX content in rabbit ocular tissues was measured from the tissues by acetonitrile based extraction. Briefly, the weighed amounts of ocular tissues were mixed with 500 μl of water containing 500 ng/ml of moxifloxacin as an internal standard, and vortexed for 15 minutes. Tissue samples were then homogenized using a hand homogenizer on an ice bath to form a uniform tissue suspension. To this tissue homogenate, acetonitrile (1.5 ml) was added and vortexed for 30 minute on a multitube vortexer (VWR LabShop, Batavia, Ill.). Precipitated tissue proteins were separated by centrifugation of the above mixture at 10,000 g for 10 min. The supernatant was pipetted out and transferred into clean glass tubes and evaporated under nitrogen stream (Multi-Evap; Organomation, Berlin, Mass.) at 40° C. The residue after evaporation was reconstituted with 500 μl of acetonitrile: water mixture (75:25 v/v) and subjected to LC-MS/MS analysis. The acetonitrile based extraction method for extraction of GFX from the rabbit ocular tissue was validated to determine the extraction recovery using three different concentrations (low, medium and high) to cover the entire range of expected concentrations of GFX in various ocular tissues.

The aqueous humor and vitreous samples were analyzed directly after dilution, without extraction. Briefly, the aqueous humor and vitreous samples were 5-fold diluted with acetonitrile containing moxifloxacin as an internal standard, vortexed for 10 min and centrifuged at 10,000 g for 5 min. The supernatant (200 μl) was transferred into LC-MS/MS vials and subjected to analysis.

Calibration curves for tissue sample analysis were developed in an appropriate blank rabbit ocular tissue using 10 concentrations by spiking a known amount of analyte and internal standard.

LC-MS/MS Analysis:

GFX concentrations in the transport study and ocular tissue samples were analyzed using a validated LC-MS/MS method. Analysis was performed using an API-3000 triple quadrupole mass spectrometry (Applied Biosystems, Foster City, Calif., USA) coupled with a PerkinElmer series-200 liquid chromatography (Perkin Elmer, Walthm, Mass., USA) system. Chromatographic separation of GFX and the internal standard moxifloxacin was performed on Obelisc C18 column (2.1×10 mm, 3 μm). Elution of analytes was performed using linear gradient elution with mobile phase consisting of 5 mM ammonium formate (pH 3.5) and acetonitrile (pH 3.5) with a flow rate of 300 μl/min and total run time of 6 min. GFX and moxifloxacin were analyzed in positive ionization mode with the following multiple reaction monitoring (MRM) transitions: 376→358 (gatifloxacin) and 402→384 (moxifloxacin).

Data Analysis:

All values in this study are expressed as mean±SD. Statistical comparison between two groups were determined using independent sample Student's t-test. Differences were considered statistically significant at the level of p<0.05.

Computational Modeling:

Homology models for rabbit OCT1, OCT2 and OCT3 were created using the comparative protein structure prediction software I-Tasser. Sequences were retrieved from the NCBI protein resource (http://www.ncbi.nlm.nih.gov/protein/). Models were imported into Discovery Studio 3.5 (Accelrys, San Diego, Calif.) as PDB files. These were then prepared using the “prepare protein” protocol to build loops, protonate and minimize the protein using CHARMm. The protein was then used with the “dock ligands (Lib Dock)” protocol. A 10 Å sphere was created around GLN447 in OCT1 and GLU447 in OCT2. For OCT3 binding sites were detected and the biggest was used to create a sphere with diameter 10.2 Å. For ease of docking GFX was combined with ARG to create a single structure to be used as a surrogate for the GFX-ARG ion pair during docking GFX-ARG was docked in each protein using the High Quality docking preferences, ‘Fast’ conformation method and steepest descent minimization was performed using CHARMm.

### Results

Solubility of GFX and Ion Pair Complex:

In vitro aqueous solubility of GFX and ion pair complexes at two different ratios were measured in PBS (pH 7.4) at 37° C. and results were summarized in the Table below. As can be seen in the Table, aqueous solubility of GFX was measured to be 5.49 mg/ml. Formation of the GFX ion pair with ARG and LYS at 1:1 molar ratio results in approximately 1.5-fold increase in aqueous solubility. A further increase in molar ratios of ARG and LYS to 1:3, results in an increase in aqueous solubility by 1.9 and 1.7-fold, respectively, when compared to GFX.

TABLE
Aqueous solubility of GFX, GFX-ARG and GFX-LYS complex
in phosphate buffer saline at pH 7.4. Data is expressed
as mean ± SD for n = 3.

| Sample Name | Aqueous solubility (mg/ml) |
|---|---|
| GFX | 5.49 ± 0.02 |
| GFX-ARG (1:1) | 8.48 ± 0.01 |
| GFX-ARG (1:3) | 10.63 ± 0.01 |
| GFX-LYS (1:1) | 8.09 ± 0.01 |
| GFX-LYS (1:3) | 9.38 ± 0.02 |

In Vitro Transport Across Rabbit Cornea and SCRPE:

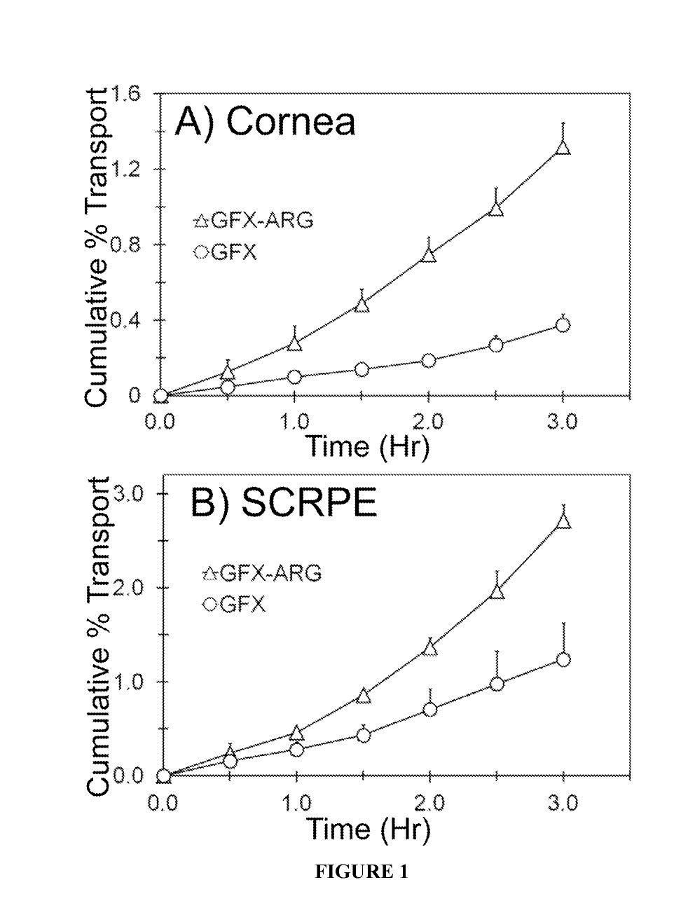

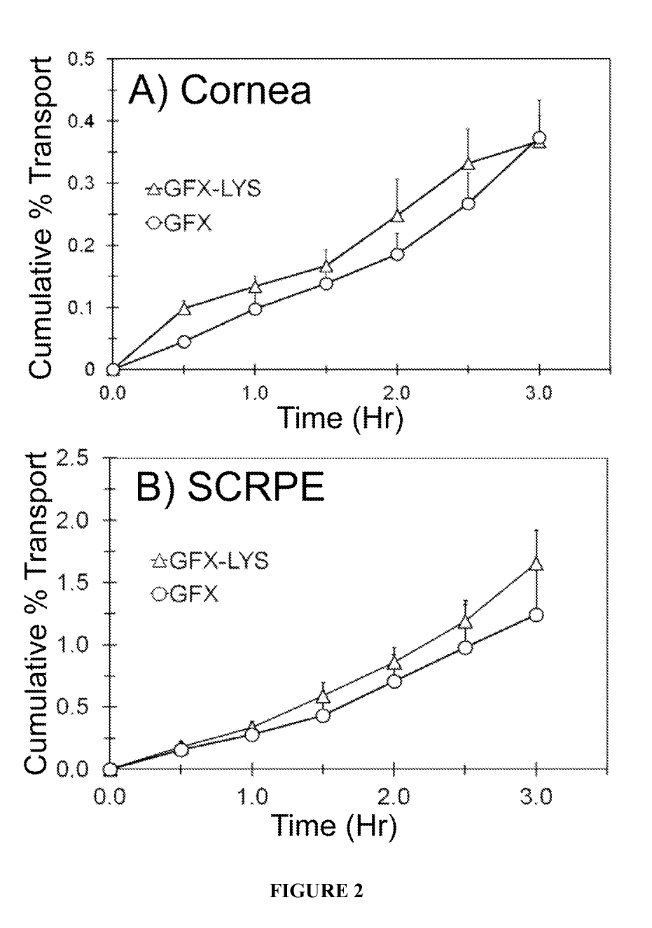

In vitro transport of GFX, GFX-ARG, and GFX-LYS across rabbit cornea and SCRPE was carried out to evaluate the effect of ion pair on permeability. As shown in FIG. 1, GFX-ARG showed a 3.5- and 2.2-fold increase in cumulative % transport as compared with GFX alone (i.e., in the absence of the counterion) across the cornea and SCRPE, respectively. In vitro transport of GFX-LYS was not significantly different from GFX across both the cornea and SCRPE. See FIG. 2.

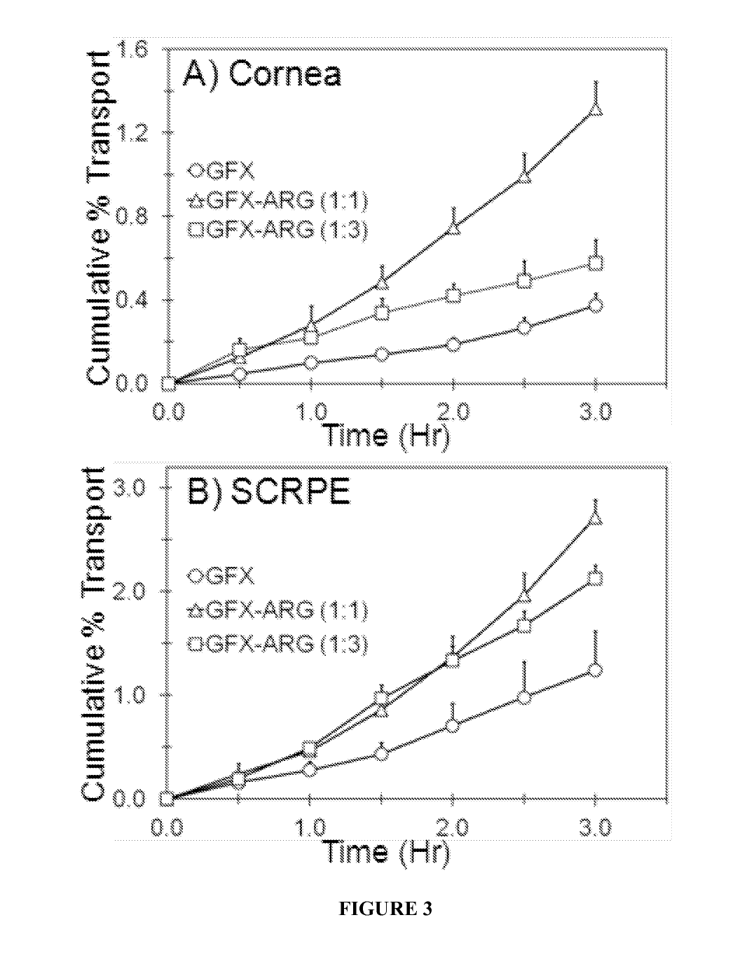

Evaluation of the effect of ARG concentration on permeability of the ion pair showed that the cumulative % transport of GFX-ARG at 1:3 molar ratios was significantly lower than 1:1 molar ratio, but significantly higher than GFX both across cornea and SCRPE respectively. See FIG. 3. These results indicate that although increasing the molar ratios of ARG in GFX-ARG ion pair increases solubility, it is not beneficial in improving the permeability. In fact, increasing the molar ratio of ARG to 1:3 resulted in decreased transport when compared with 1:1 ratio of GFX and ARG. Without being bound by any theory, it is believed that such observation may be due to competitive inhibition of transport by excess of ARG.

Identification of the Transporter Involved in Active Transport of the GFX-ARG Ion Pair:

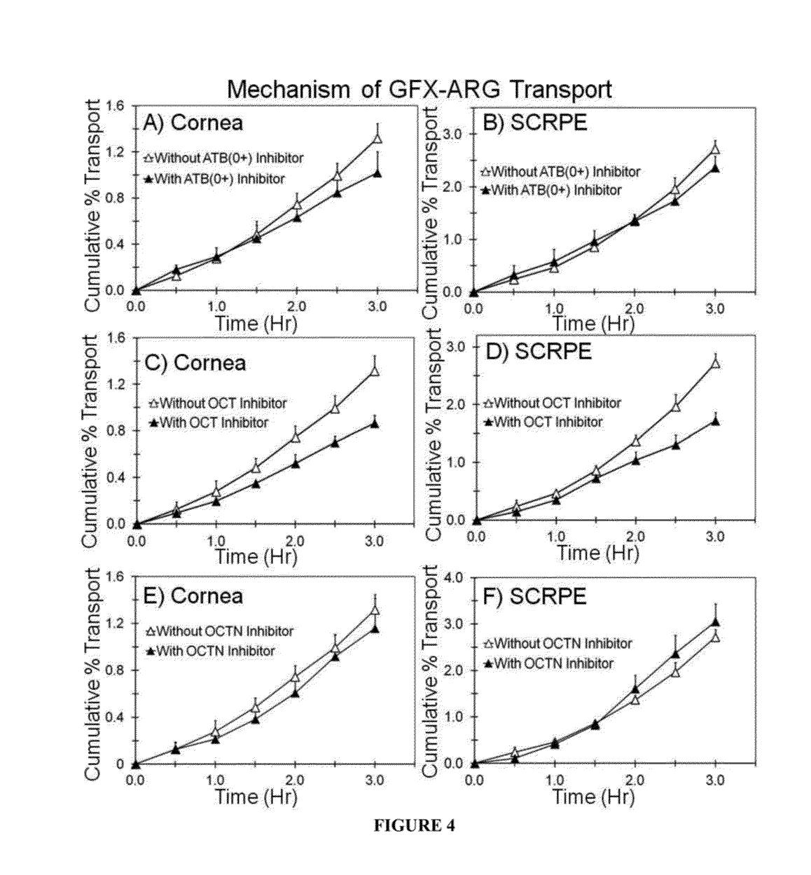

To ascertain the mechanism of enhanced permeability of the GFX-ARG ion pair, transport of GFX-ARG was carried out in the presence of specific transporter inhibitors. As the GFX-ARG ion pair complex involved an amino acid, transport was carried out in presence and absence of the ATB⁰⁺ inhibitor, α-methyl tryptophan, to elucidate the role of amino acid transporters in transport of GFX-ARG across the cornea and SCRPE. As shown in FIGS. 4A and 4B, there was no significant difference in cumulative % transport of GFX-ARG across both cornea and SCRPE in the presence and absence of α-methyl tryptophan (500 μM).

Transporter studies were also carried out in the presence of OCT and OCTN inhibitors. Transport of the GFX-ARG ion pair across rabbit cornea and SCRPE was significantly inhibited by competitive inhibition in the presence of the OCT substrate MPP indicating an involvement of the OCT transporter in GFX-ARG transport. See FIGS. 4A and 4B. However, the OCTN inhibitor L-carnitine did not show any significant inhibitory effect on the transport of GFX-ARG across both the cornea and SCRPE. See FIGS. 4E and 4F.

Comparison of In Vivo Ocular Delivery of GFX and GFX-ARG Ion Pair:

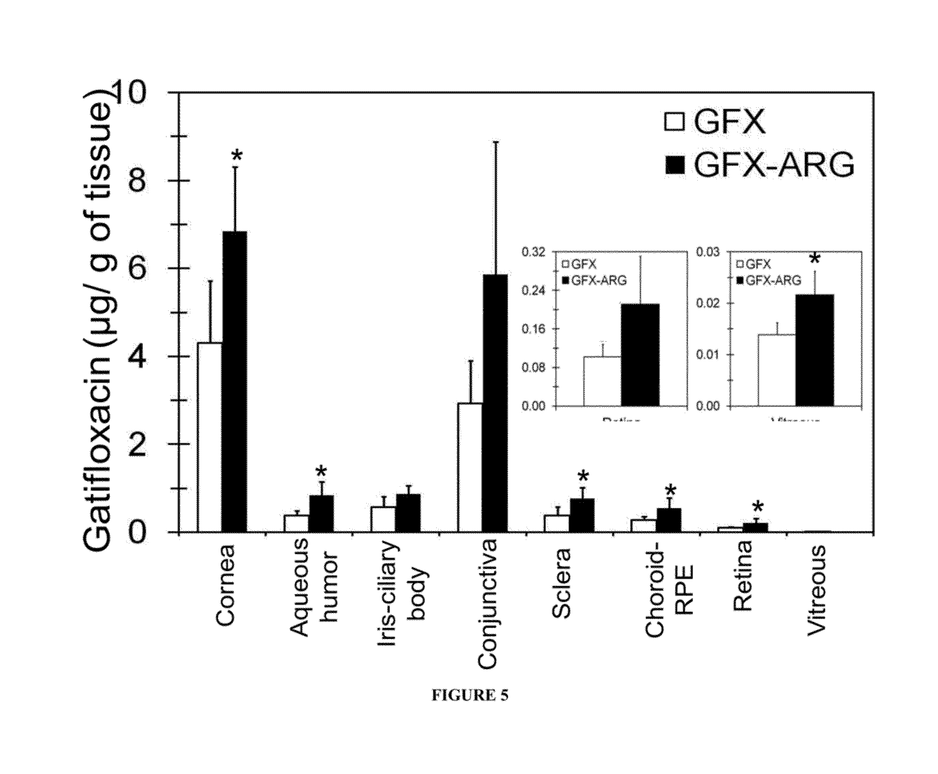

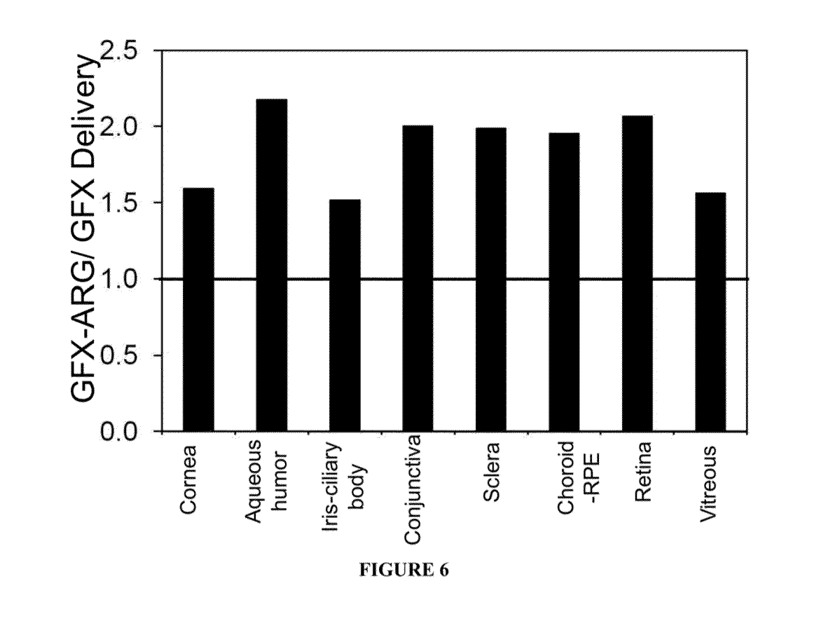

To evaluate the influence of the GFX-ARG ion pair on intraocular delivery, an in vivo ocular tissue distribution study was conducted in pigmented rabbits after topical dosing and compared with topical administration of GFX alone (i.e., without any added known ion pair). Comparison of in vivo ocular tissue distribution of GFX and the GFX-ARG ion pair after topical application is shown in FIG. 5. As can be seen in FIG. 5, GFX ocular tissue levels were significantly higher for GFX-ARG ion pair compared with GFX alone. As shown in FIG. 6, GFX-ARG showed 1.5 to 2.2-fold increase in delivery to all ocular tissues when compared with GFX alone, which is in agreement with the in vitro transport results.

Computational Modeling:

Within the top 10 crystal structure templates used for homology modeling for each OCT were 1PW4, 2GFP and 3O7Q. The I-Tasser model parameters for the rabbit OCT1 homology model were C-score=−2.44, estimated accuracy: 0.43±0.14 (TM-score) 13.5±4.0 Å (RMSD). The I-Tasser model parameters for the rabbit OCT2 homology model were C-score=−2.26, estimated accuracy of: 0.45±0.14 (TM-score) 13.1±4.2 Å (RMSD). The I-Tasser model parameters for the rabbit OCT3 homology model were C-score=−0.77, estimated accuracy of: 0.62±0.14 (TM-score) 8.6±4.5 Å (RMSD). Where C-score is a confidence score for estimating the quality of predicted models by I-TASSER in which a higher value signifies a model with a high confidence. TM-score and RMSD are known standards for measuring structural similarity between two structures that are used to measure the accuracy of structure modeling when the native structure is known. In this case these are predicted values. A TM-score >0.5 indicates a model of correct topology and a TM-score <0.17 means a random similarity. The LibDock score for docking ARG-GFX in each transporter was similar for the best pose: 106.9 (OCT1), 111 (OCT2) 116 (OCT3).

### Discussion

Transporter mediated delivery of the GFX ion pair complexes with the amino acids ARG and LYS was evaluated. Some of the key findings of the present disclosure are that: (1) formation of the GFX ion pair complexes with ARG or LYS resulted in an increase in aqueous solubility; (2) GFX-ARG showed a significantly improved permeability across rabbit cornea and SCRPE; (3) transport of GFX-ARG across rabbit cornea and SCRPE was inhibited significantly in the presence of the OCT inhibitor MPP⁺, indicating a role for OCT transporters in the flux of GFX-ARG; and (4) GFX-ARG showed 1.5-2.2 fold higher in vivo delivery to all ocular tissues when compared with GFX alone, and enhanced drug delivery to the back of the eye tissues.

Ion pair mediated enhanced delivery for poorly permeable drugs after oral administration has been extensively studied. Typically in oral administration, lipophilic counterions are commonly used with poorly permeable highly charged polar drug molecules, forming lipophilic ion pair complexes to increase the passive diffusion. This approach allows enhancement of the permeability of poorly permeable drugs and removes the need for a prodrug strategy or other permeability enhancers. Unfortunately, this strategy has shown only a limited applicability for poorly permeable lipophilic or amphiphilic molecules. Introduction of functional groups with the ion pair method, to make the drug a substrate for a transporter, can act as an alternative strategy to increase the transporter mediated transport for poorly permeable amphiphilic molecules. Previous studies showed that biliary excretion of high molecular weight organic cations such as tributylmethyl-ammonium is mediated by P-glycoprotein efflux after formation of ion pair complex with the bile acid taurodeoxycholate.

However, to date there has been no similar ion-pair mediated uptake transport of solutes and complexes. Surprisingly and unexpectedly, the present inventors have discovered that the role of uptake transporters OCT in ocular delivery of GFX ion pair complexes significantly increased the topical ocular drug delivery. GFX was used as one of the test drugs for its hydrophilic amphiphilic nature and its potential ability to form ion pair complexes with counterion amino acids such as ARG and LYS. ARG and LYS are cationic amino acids and carry a positive charge at physiological pH and form ion pair complexes with the carboxylic acid group of GFX. The present inventors believed that this ion pair would have a strong aqueous binding constant (K_{11aq}=100-1000 M⁻¹) to prevent a substantial dissociation of the ion pair during membrane transport. Indeed, GFX and counterions showed very strong aqueous binding constants (K_{11aq}=˜100-200 M⁻¹) for both ARG and LYS with GFX.

The ability of ARG and LYS to enhance the permeability of GFX was also evaluated using in vitro transport studies across isolated rabbit cornea and SCRPE using a modified Ussing chamber assembly. Amino acid transporters including the L-type amino acid transporter (LAT), cationic amino acid transporters (e.g., hCAT1) and ATB⁰⁺ have abundant expression in cornea and RPE. Initially, it was believed by the present inventors that the GFX-ARG and GFX-LYS ion pair complexes are suitable substrates for these amino acid transporters and would be actively transported across ocular barriers by these transporters. However, an in vitro transport study of GFX-ARG and GFX-LYS across rabbit cornea and SCRPE showed that only GFX-ARG significantly improved permeability across both tissues compared to GFX alone. These in vitro transport results indicate that the transport of the ion pair is not significantly mediated by amino acid transporters. Surprisingly and unexpectedly, further elucidation study of the transport mechanisms for enhanced permeability of GFX-ARG showed that its transport across ocular barriers was actually mediated at least in part by the OCT transporters and not significantly by the amino acid transporters and carnitine transporters.

The present inventors believed that since ARG has a guanidine group (pKa=12.5) in its structure that can impart a cationic charge to the ion pair complex, it can be a substrate for OCT transporters. Indeed, evaluation of the effect of ARG concentration on transport of the GFX-ARG ion pair showed that the cumulative % transport of GFX-ARG decreased with an increase in ARG concentration from 1:1 to 1:3 molar ratios. Without being bound by any theory, it is believed that in such cases the decrease in cumulative % transport with an increase in ARG concentration is due to the competitive inhibition of GFX-ARG transport by an excess of ARG which competes with binding of the ion pair to the OCT transporters. Other drugs that are taken up in the rabbit cornea by OCTs include tilisolol and the like.

To demonstrate the effectiveness of the GFX-ARG ion pair for topical ocular drug delivery, in vivo ocular delivery experiments were performed in normal pigmented rabbits using clear aqueous solution of the GFX-ARG ion pair and the results were compared with GFX in the absence of ARG in the same solution (i.e., GFX alone). Drug levels were compared at the end of 1 hr because the present inventors have discovered the peak drug concentrations in posterior ocular tissues after topical application were at around 1 hr post dosing. Since the present inventors have discovered that GFX-ARG is transported at least in part by OCT transporters across ocular barriers, GFX delivery to the intraocular tissues was expected to be higher for the GFX-ARG ion pair than GFX alone. As expected, in vivo ocular tissue distribution study showed that the GFX-ARG had higher concentrations of GFX in all ocular tissues than GFX without the ion pair (i.e., in the absence of ARG). The present inventors have also observed increased ocular delivery of GFX with GFX-guanidine g6 dendrimer compared to GFX alone. However, this latter observation was due to a 4-fold increase in aqueous solubility of GFX by guanidine g6 dendrimer. In the present disclosure, the dosing concentrations of GFX-ARG and GFX were identical (5 mg/ml); therefore, the observed differences in ocular drug levels are not due to concentration dependent flux but the increased transporter mediated uptake and permeability.

Computer simulations showed that the GFX-ARG structure used as a surrogate for the GFX-ARG ion pair fits within the binding site for both OCT1 and OCT2 homology models using residue 447 as the approximate binding site centroid. In silico data showed GFX-ARG interacts with residues previously identified in OCTs. While OCT1 and OCT2 appear to have the GFX-ARG docked in different orientations, the guanidine portion interacts with residue 241 in both transporters. GFX-ARG also appears to dock into OCT3. The computer simulated docking data of GFX-ARG in homology models provides additional evidence to it being a substrate for at least OCT1 and OCT2 in rabbit cornea and SCRPE.

### Conclusion

Topical drug delivery to the intraocular tissues is restricted by poor permeability across ocular barriers. Utilization of uptake drug transporters present in ocular barriers is helpful in improving uptake of poorly permeable hydrophilic drugs. In this study, using GFX as a model amphiphillic drug with antibacterial effects in the treatment of ocular infectious disease, the present inventors have shown that the intraocular delivery of GFX can be significantly enhanced in vitro and in vivo through formation of an ion pair complex with ARG. Results from this study provides new insights into the underlying mechanisms for enhanced delivery with an ion pair for poorly permeable drugs. Utilization of this approach with proper selection of counterions for transporter guided drug delivery in topical drug delivery to the anterior eye tissues can be used effectively to treat various ocular diseases and clinical conditions associated with ocular tissues.

### Example 2

Human Eyes and Tissue Specimens:

For transport studies, human cadaver eyes were obtained from the Rocky Mountain Lions Eye Bank (Aurora, Colo.) within 48 hrs of death. For immunohistochemical analysis of transporters, human ocular tissue specimens were obtained from archives of University of Colorado, Anschutz Medical Campus eye pathology laboratory. The summary of patient data including age, sex, condition of eye and reason for death are provided in the following Table:

TABLE
Patient demographic information.

| Pa- | | | | | | |
|---|---|---|---|---|---|---|
| tient | Experiment | | | | Lens | Death |
| ID | performed | Sex | Age | Race | Status | Cause |
| 01 | Transport | Male | 69 | Caucasian | Phakic | Renal |
| | | | | | | Disease |
| 02 | Transport | Female | 56 | Caucasian | Phakic | Cerebro- |
| | | | | | | vascular |
| | | | | | | Accident |
| 03 | Transport | Male | 65 | Caucasian | Phakic | Myocardial |
| | | | | | | Infraction |
| 04 | Transport | Male | 83 | Caucasian | Aphakic | Renal |
| | | | | | | Failure |
| 06 | IHC | Female | 52 | Caucasian | Phakic | Not known |
| 07 | IHC | Male | 62 | Caucasian | Phakic | Diabetes |
| 08 | IHC | Male | 64 | Caucasian | Aphakic | Heart |
| | | | | | | Attack |

For transport study, the eyes were immediately used upon arrival. For immunohistochemistry, formalin-fixed paraffin embedded 5 μm thick sections of whole human eyes were obtained from the archives of the University of Colorado Eye Pathology Laboratory.

Immunohistochemical Analysis:

For immunohistochemical staining, formalin-fixed paraffin embedded 5 μm thick sections were obtained from whole human eye and mounted on (3-aminopropyl)triethoxysilane-treated slides. The slides were deparaffinized in xylene for 20 min to remove the embedding paraffin media and washed with absolute ethanol. Slides were gradually rehydrated using series of alcohol washes, including 95%, 90%, 70% and 50% and distilled water for 5 min each. Endogenous peroxides activity was blocked by incubating the slides with 3% H₂O₂ in absolute methanol for 15 min at 37° C. Whenever necessary antigen retrieval was performed by incubating the slides in boiling 10 mM citrate buffer (pH 6.0) or 10 mM Tris-HCL containing 1 mM of EDTA (pH 9.0) at 95° C. for 20 min. After antigen retrieval, slides were washed and permeablized with phosphate buffer saline (PBS) containing 0.1% Triton X-100 (PBS-T). Nonspecific antibody binding was blocked by incubating the slides with blocking buffer (1.0% BSA and 10% goat serum in PBS). Tissue sections were then incubated with appropriate dilution of primary antibody in PBS-T at 37° C. for 1 hr or at 4° C. overnight. Summary of primary antibody dilution, incubation condition, antigen retrieval procedure, secondary antibody and detection system used are provided in the following Table:

TABLE
Summary of antibodies and conditions for immunohistochemistry
of drug transporter in human ocular tissues.

| Trans- | | | Incubation |
|---|---|---|---|
| porter | Primary Antibody (source) | Dilution | Condition |
| PEPT-1 | Antihuman goat PEPT-1 Ab | 1:200 | 60 min |
| | (Santa Cruz Biotech, Santa Cruz, CA) | | at 37° C. |
| PEPT-2 | Antihuman rabbit PEPT-2 Ab | 1:200 | Overnight |
| | (Santa Cruz Biotech, Santa Cruz, CA) | | at 4° C. |
| OCT-1 | (Santa Cruz Biotech, Santa Cruz, CA) | 1:200 | Overnight |
| | | | at 4° C. |
| OCT-2 | (Santa Cruz Biotech, Santa Cruz, CA) | 1:200 | 60 min |
| | | | at 37° C. |
| ATB⁰⁺ | Antihuman rabbit ATB⁰⁺ Ab | 1:5000 | 60 min |
| | (Medical and Biological Laboratories, | | at 37° C. |
| | Japan) | | |
| MCT-1 | (Santa Cruz Biotech, Santa Cruz, CA) | | |
| MCT-3 | Antihuman rabbit MCT3 Ab | 1:200 | 60 min |
| | (Santa Cruz Biotech, Santa Cruz, CA) | | at 37° C. |

| Trans- | Secondary | |
|---|---|---|
| porter | Antibody | Antigen Retrieval Procedure |
| PEPT-1 | Poly-AP | High pH heat induced antigen |
| | antigoat IgG | retrieval (10 mM Tris-HCl, 1 mM |
| | | EDTA, pH 9.0 at 95° C. for 20 min) |
| PEPT-2 | Poly-AP | Low pH heat induced antigen |
| | antirabbit IgG | retrieval (10 mM citrate buffer, |
| | | pH 6.0 at 95° C. for 20 min) |
| OCT-1 | Poly-AP | High pH heat induced antigen |
| | antirabbit IgG | retrieval (10 mM Tris-HCl, 1 mM |
| | | EDTA, pH 9.0 at 95° C. for 20 min) |
| OCT-2 | Poly-AP | High pH heat induced antigen |
| | antirabbit IgG | retrieval (10 mM Tris-HCl, 1 mM |
| | | EDTA, pH 9.0 at 95° C. for 20 min) |
| ATB⁰⁺ | Poly-AP | High pH heat induced antigen |
| | antirabbit IgG | retrieval (10 mM Tris-HCl, 1 mM |
| | | EDTA, pH 9.0 at 95° C. for 20 min) |
| MCT-1 | Poly-AP | Low pH heat induced antigen |
| | antirabbit IgG | retrieval (10 mM citrate buffer, |
| | | pH 6.0 at 95° C. for 20 min) |
| MCT-3 | Poly-AP | No antigen retrieval |
| | antirabbit IgG | |

After incubation with primary antibody, sections were washed three times with PBS-T and incubated with appropriate dilution of alkaline phosphates linked-secondary antibody (Leica; Bond™ Polymer Refine Red Detection) in Tris buffer saline for 30 min. After further wash of sections with PBS-T, sections were stained and visualized using VECTOR® Red alkaline phosphatase detection system (Vector Laboratories; Vector® Red Alkaline Phosphatase Substrate Kit I) for 5 minutes. The slides were counterstained with hematoxylin (Auto Hematoxylin; Open Biosystems) for 30 second to stain the nuclei. For control experiments, the sections were processed same as above except the incubation step with primary antibody was omitted.

In Vitro Transport Across Human Cornea and Sclera-Choroid-RPE:

In vitro transport studies across human cornea and sclera-choroid-RPE (SCRPE) were carried out according to previously published method using the cassette dosing approach. Briefly, cassette of drug transporter substrate including Gly-Sar (PEPT), L-tryptophan (ATB⁰⁺), MPP⁺ (OCT), and phenyl acetic acid (MCT) at concentration of 100 μM in assay buffer was prepared. Briefly, the human eyes were washed with assay buffer and cleaned from muscle and conjunctiva tissues, and anterior and posterior parts were separated by giving circumferential cut behind the limbus. Small sclera part was left with cornea, which helps in mounting of cornea on Ussing chambers. Neural retina was separated from the choroid-RPE by filling the eye cup with assay buffer, and floating retina was collected. After separation of retina, eye cup was divided into two rectangular pieces (˜1.5×1.5 cm) of sclera-choroid-RPE. Isolated tissues were mounted on modified Ussing chambers (Navicyte, Sparks, Nev.) such that the episcleral side of SCRPE or epithelial side of cornea was facing the donor chamber and retinal side or endothelial side of cornea is facing the receiver chamber. To study the effect of directionality on transport, one set of Ussing chambers mounted so that sclera side facing the donor side and another set was mounted so that choroid-RPE facing the donor side. The effect of directionality of transport across cornea was not evaluated. The chambers were filled with 1.5 ml of assay buffer with (donor side) or without (receiver side) the cocktail of drug transporter substrates. For study of effect of transporter inhibitors, cocktail mixture (500 μM) of transporter inhibitor was added on both donor and acceptor side. Summary of specific transporter substrates and inhibitor used for transport study are provided in the following Table:

TABLE
List of transporter, specific substrates and inhibitors
for particular transporter, and inhibition mechanism.

| Trans- | Specific | Specific | |
|---|---|---|---|
| porter | Substrate | Inhibitor | Inhibition Mechanism |
| PEPT | Gly-Sar | H-Pro-Phe-OH | Competitive Inhibition |
| OCT | MPP+ | Metformin | Competitive Inhibition |
| ATB⁰⁺ | L-Tryptophan | α-Methyl | Specific Inhibition |
| | | Tryptophan | |
| MCT | Phenyl Acetic | Nicotinic acid | Competitive Inhibition |
| | Acid | | |

During the transport study, the bathing fluids were maintained at 37° C. using a circulating warm water and pH 7.4 using 95% air-5% CO₂ aeration. Samples were collected (200 μL) form receiver side every hour for 6 hr and the lost volume was compensated with fresh assay buffer pre-equilibrated at 37° C. The drug levels were analyzed using a LC-MS/MS assay. Permeation data were corrected for dilution of the receiver solution with sample volume replenishment.

LC-MS/MS Analysis:

Analytes concentrations in transport study samples were analyzed using LC-MS/MS method after 5-fold dilution with acetonitrile to reduce the salt concentrations. Cassette analysis method was developed for simultaneous analysis of Gly-Sar, L-tryptophan and MPP⁺. Phenyl acetic acid was analyzed separately in negative ionization method and normal phase separation method. An API-3000 triple quadrupole mass spectrometry (Applied Biosystems, Foster City, Calif., USA) coupled with a PerkinElmer series-200 liquid chromatography (Perkin Elmer, Walthm, Mass., USA) system was used for analysis. Gly-Sar, L-tryptophan and MPP⁺ were separated on Supelco C-5 column (2.1×10 mm, 3 μm) using water containing 0.1% formic acid (A) and acetonitrile:methanol (50:50 v/v) containing 0.1% formic acid (B) as mobile phase and a linear gradient elution at a flow rate of 0.3 ml/min with total run time of 9 min. Phenyl acetic acid was separated in normal phase separation mode using Obelisc-N silica column (2.1×10 mm, 3 μM) using 5 mM ammonium formate, pH 3.5 (A) and acetonitrile (B) as mobile phase in linear gradient mode at flow rate of 0.3 ml with total run time of 6 min. Gly-Sar, L-tryptophan, and MPP⁺ were analyzed in positive ionization mode with following multiple reaction monitoring (MRM) transitions: 147→90 (Gly-Sar); 205→188 (L-tryptophan); 170→128 (MPP⁺). Phenyl acetic acid was analyzed in negative ionization mode with following multiple reaction monitoring (MRM) transitions: 135→91 (Phenyl acetic acid).

Data Analysis:

All values in this study are expressed as mean±s.d. Statistical comparison between two groups were determined using independent sample Student's t-test. Differences were considered statistical significant at the level of p<0.05.

### Compounds/Prodrugs

Exemplary compounds/prodrugs that are useful in methods of the invention and the corresponding target transporters are listed in the following Table:

| | | Transporter | % Cumulative transport | | |
|---|---|---|---|---|---|
| Parent drug | Prodrug | targeted | Cornea | Conjunctiva | SCRPE |
| Gatifloxacin | None | | 0.37 ± 0.059 | 9.78 ± 1.15 | 0.191 ± 0.04 |
| | | | (Rabbit) | (Rabbit) | (Bovine); |
| | | | | | 1.24 ± 0.38 |
| | | | | | (Rabbit) |
| Gatifloxacin | GFX-ARG | ATB | 0.80 ± 0.16 | (N/D) | 2.40 ± 0.19 |
| | | | (Rabbit) | | (Rabbit) |
| Gatifloxacin | GFX-LYS | ATB | N/D | N/D | N/D |
| Gatifloxacin | GFX-LEU | ATB | 0.56 ± 0.15 | 9.76 ± 3.02 | 1.55 ± 0.51 |
| | | | (Rabbit) | (Rabbit) | (Rabbit) |
| Gatifloxacin | GFX-OCT | OCT | 0.63 ± 0.14 | 18.9 ± 2.80 | 0.358 ± 0.08 |
| | | | (Rabbit) | (Rabbit) | (Bovine); |
| | | | | | 2.07 ± 0.27 |
| | | | | | (Rabbit) |
| Gatifloxacin | GFX-MCT | MCT | 0.49 ± 0.035 | 16.57 ± 2.26 | 2.99 ± 0.26 |
| | | | (Rabbit) | (Rabbit) | (Rabbit) |
| Celecoxib | None | | N/D | N/D | 0.114 ± 0.034 |
| | | | | | (Bovine) |
| Celecoxib | CXB-ARG | ATB | N/D | N/D | N/D |
| Celecoxib | CXB-LYS | ATB | N/D | N/D | N/D |
| Celecoxib | CXB-LEU | ATB | N/D | N/D | N/D |
| Celecoxib | CXB-OCT | OCT | 0.199 ± 0.048 | N/D | 0.368 ± 0.06 |
| | | | (Bovine) | | (Bovine) |
| Celecoxib | CXB-MCT | MCT | N/D | N/D | N/D |

| | | Water (water for injection) |
|---|---|---|
| Parent drug | Prodrug | solubility (mg/ml) |
| Gatifloxacin | None | 2.5 |
| Gatifloxacin | GFX-ARG | 25.50 |
| Gatifloxacin | GFX-LYS | 48.00 |
| Gatifloxacin | GFX-LEU | 2.16 |
| Gatifloxacin | GFX-OCT | 33.26 |
| Gatifloxacin | GFX-MCT | 3.01 |
| Celecoxib | None | 0.002 to 0.007 |
| Celecoxib | CXB-ARG | 0.035 |
| Celecoxib | CXB-LYS | 0.05 |
| Celecoxib | CXB-LEU | 0.016 |
| Celecoxib | CXB-OCT | 3.22 |
| Celecoxib | CXB-MCT | 0.620 |

### Synthesis of some of the representative compounds that are used as counterions or are bonded to the drug are provided below. It should be appreciated that the scope of the invention is not limited to these particular compounds as the scope of the invention includes any compounds that can be used as counterions to target the desired transporter, such as ATB^{0,+}, OCT, MCT, etc.

### Synthesis of 2-amino-5-guanidino-N-((4-(5-p-tolyl)-3-(trifluoromethyl)-1H-pyrazol-1-yl)phenyl)sulfonyl)pentanamide (CXB-ARG)

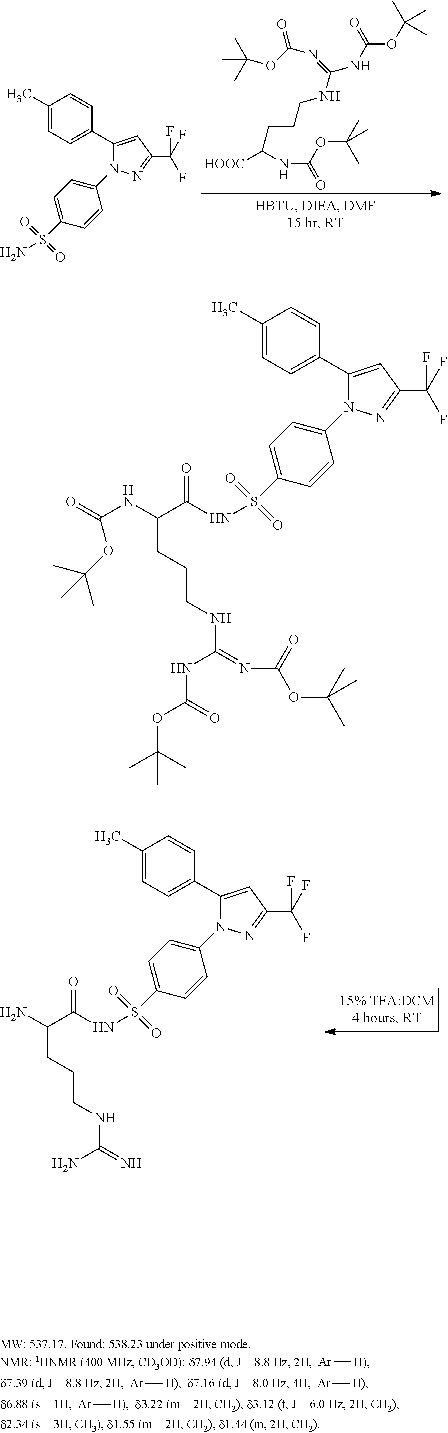

Step 1:

84.0 mg (0.17 mmol) of (E)-5-(2,3-bis(tert-butoxycarbonyl)guanidino)-2-((tert-butoxycarbonyl)amino)pentanoic acid, 120.0 mg (0.31 mmol) of HBTU (O-Benzotriazole-N,N,N′,N′-tetramethyl-uronium-hexafluoro-phosphate), and 75.0 μl (0.30 mmol) of DIEA (N,N-Diisopropylethylamine) were dissolved in anhydrous 3.0 ml DMF (dimethyl formamide). The reaction was stirred for 1 hour at room temperature under inert gas (argon). After 1 hour 95.0 mg of celecoxib (0.25 mmol) was added to the reaction under argon and the resulting reaction were stirred for 15 hours at room temperature. At the end of the reaction, the solvent was evaporated and the residue was purified by flash column chromatography. Yield of the product was 50%.

Step 2:

55 mg of the product from step 1 was dissolved in 3.0 ml of 15:85 trifluoroacetic acid: dichloromethane and the reaction was stirred for 3 hours at room temperature. Once the reaction was completed, solvent was evaporated and the residue was dried in vacuum for few hours to completely evaporate residual TFA. Finally, the product was purified using amine bonded silica column chromatography. Yield of the product was 60%.

### Solubility:

| | Measured | Predicted by ACD |
|---|---|---|
| Drug | (mg/ml) | PhysChem software v 12.0 |
| Celecoxib | 0.002 | 0.014 |
| CXB-ARG | 0.035 | 0.15 |

### Synthesis of 2-amino-4-methyl-N-((4-(5-p-tolyl)-3-(trifluoromethyl)-1H-pyrazol-1-yl)phenyl)sulfonyl)pentanamide [CXB-ATB (Leucine)]

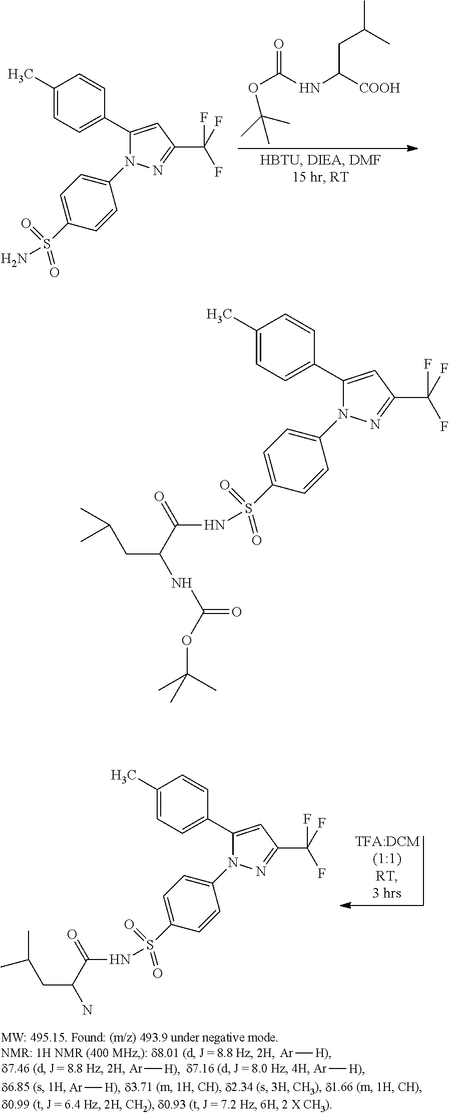

Step 1

46.0 mg (0.24 mmol) of 2-((tert-butoxycarbonyl)amino)-4-methylpentanoic acid, 112.0 mg (0.3 mmol) of HBTU (O-Benzotriazole-N,N,N′,N′-tetramethyl-uronium-hexafluoro-phosphate), and 52.0 μl (0.30 mmol) of DIEA (N,N-Diisopropylethylamine) were dissolved in anhydrous 3.0 ml DMF (dimethyl formamide). The reaction was stirred for 1 hour at room temperature under inert gas (argon). After 1 hour 75.0 mg of celecoxib (0.2 mmol) was added to the reaction under argon and the resulting reaction was stirred for 15 hours at room temperature. At the end of the reaction, the solvent was evaporated and the product was purified by using flash column chromatography. Yield of the product was 38%.

Step 2:

35 mg of the product from step 1 was dissolved in 3.0 ml of 1:1 mixture of trifluoroacetic acid:dichloromethane and the reaction was stirred for 3 hours at room temperature. Once the reaction was completed, solvent was evaporated and the residue was dried in vacuum for few hours to completely evaporate residual TFA. Finally, the product was purified by column chromatography. Yield of the product was 80%.

### Synthesis of 2,6-diamino-N-(4-(5-p-tolyl-3-(trifluoromethyl)-1H-pyrazol-1-yl)phenylsulfonyl)hexanamide (CXB-LYS)

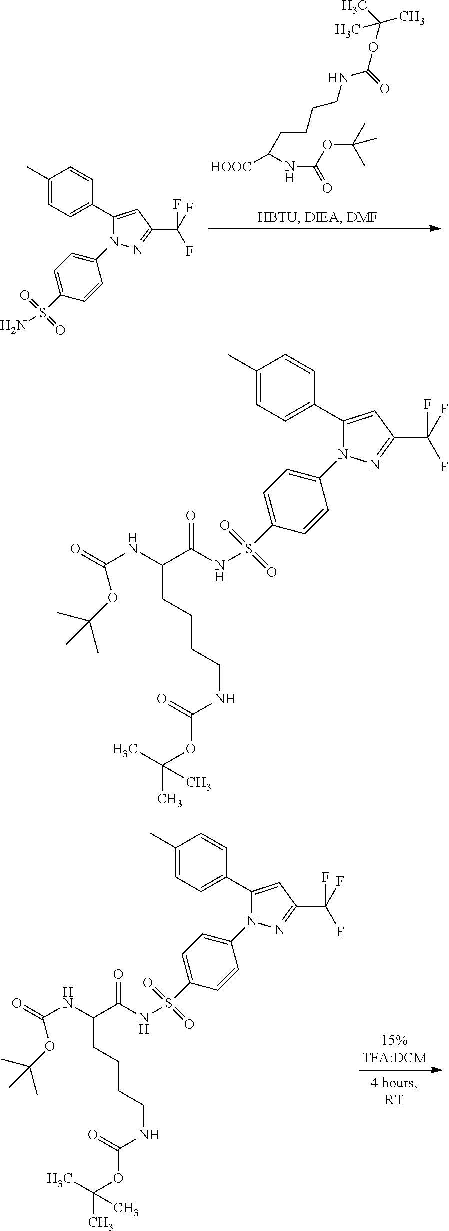

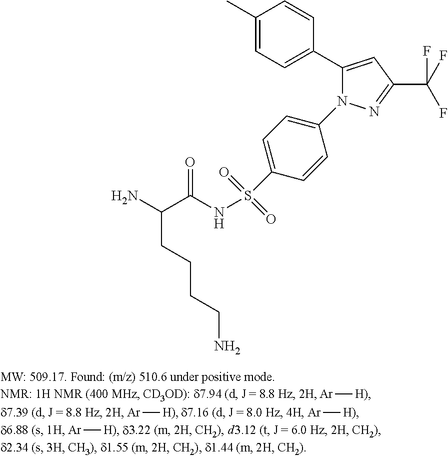

Step 1:

84.0 mg (0.17 mmol) of 2,6-bis((tert-butoxycarbonyl)amino)hexanoic acid, 120.0 mg (0.31 mmol) of HBTU (O-Benzotriazole-N,N,N′,N′-tetramethyl-uronium-hexafluoro-phosphate), and 75.0 μl (0.30 mmol) of DIEA (N,N-Diisopropylethylamine) were dissolved in anhydrous 3.0 ml DMF (dimethyl formamide). The reaction was stirred for 1 hour at room temperature under inert gas (argon). After 1 hour 95.0 mg of celecoxib (0.25 mmol) was added to the reaction under argon and the resulting reaction were stirred for 15 hours at room temperature. At the end of the reaction, the solvent was evaporated and the product was purified by flash column chromatography. Yield of the product was 50%.

Step 2:

55 mg of the product from step 1 was dissolved in 3.0 ml of 15:85 trifluoroacetic acid: dichloromethane and the reaction was stirred for 3 hours at room temperature. Once the reaction was completed, solvent was evaporated and the residue was dried in vacuum for few hours to completely evaporate residual TFA. Finally, the product was purified using amine bonded silica column chromatography. Yield of the product was 60%.

### Solubility:

| | Measured | Predicted by ACD |
|---|---|---|
| Drug | (mg/ml) | PhysChem software v 12.0 |
| Celecoxib | 0.002 | 0.014 |
| CXB-LYS | 0.05 | 0.15 |

### Synthesis of 5-oxo-5-(4-(5-(p-tolyl)-3-(trifluoromethyl)-1H-pyrazol-1-yl)phenylsulfonamido)pentanoic acid (CXB-MCT)

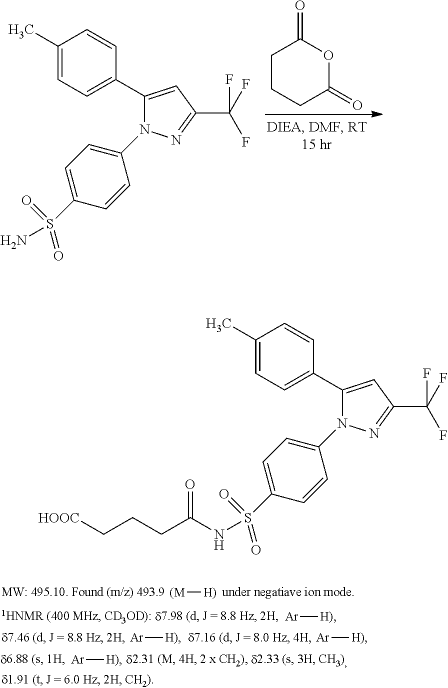

100 mg (0.26 mmol) of Celecoxib, 114.0 mg of glutaric anhydride (0.33 mmol), and 68 μl of DIEA (N,N-Diisopropylethylamine) were dissolved in 3.0 ml of anhydrous DMF and the reaction was stirred at room temperature for 15 hours. The reaction progress was monitored by TLC. Once the reaction was completed, the solvent was evaporated under vacuum. Product was purified by flash column chromatography. Yield of the reaction was 89%.

### Synthesis of 4-(dimethylamino)-N-((4-(5-(p-tolyl)-3-(trifluoromethyl)-1H-pyrazol-1-yl)phenyl)sulfonyl)butanamide (CXB-OCT)

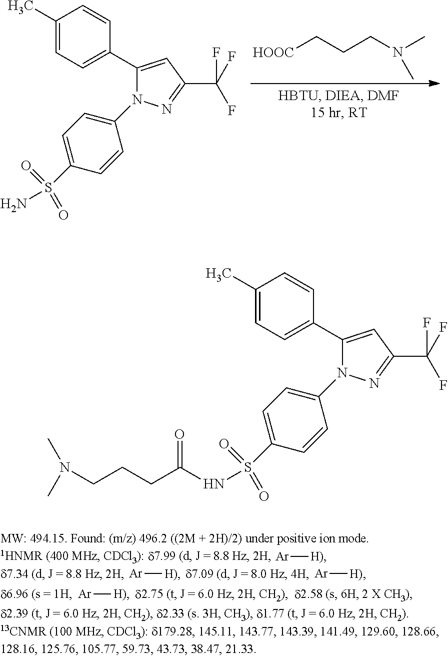

30.0 mg (0.18 mmol) of 4-(dimethylamino)butanoic acid, 101.0 mg (0.27 mmol) of HBTU (O-Benzotriazole-N,N,N′,N′-tetramethyl-uronium-hexafluoro-phosphate), and 52.0 μl (0.30 mmol) of DIEA (N,N-Diisopropylethylamine) were dissolved in anhydrous 3.0 ml DMF (dimethyl formamide). The reaction was stirred for 1 hour at room temperature under argon. After 1 hour Celecoxib (0.2 mmol) was added to the reaction and the resulting reaction was stirred for 15 hours at room temperature. At the end of the reaction, the solvent was evaporated and the product was purified by flash column chromatography. Yield of the product was 39%.

### Synthesis of 7-(4-(2-amino-5-guanidinopentanoyl)-3-methylpiperazin-1-yl)-1-cyclopropyl-6-fluoro-8-methoxy-4-oxo-1,4-dihydroquinoline-3-carboxylic acid (GFX-ARG)

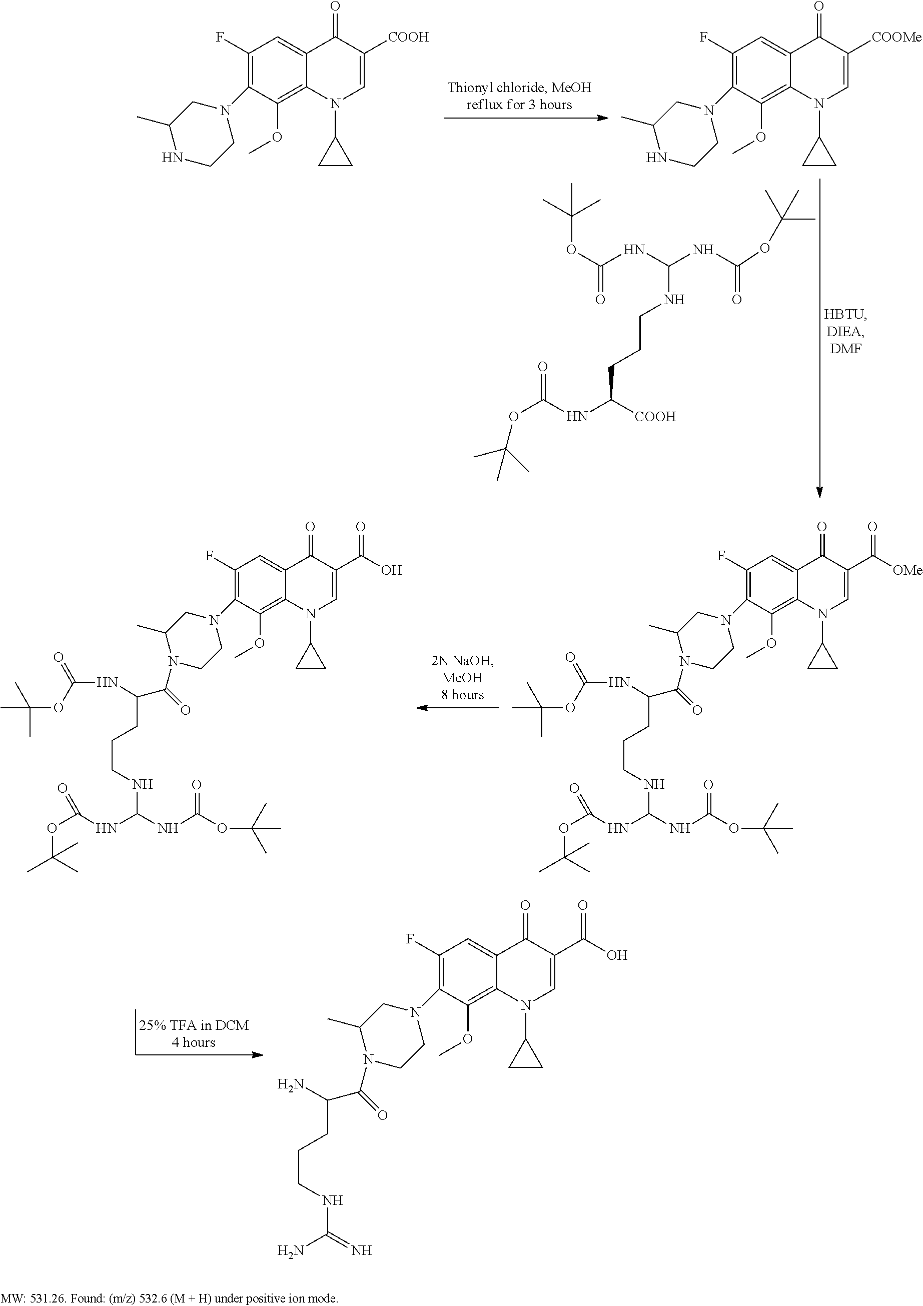

Step 1:

250 mg of gatifloxacin was dissolved in 5.0 ml of HPLC grade methanol and the reaction flask was cooled to 0° C. Subsequently, 0.052 ml of thionyl chloride was added dropwise to the reaction mixture and the reaction contents were slowly brought to room temperature. Then the reaction was refluxed for 24 hours. Once the reaction was completed, the solvent was evaporated and the contents were dried under high vacuum to get rid of the excess of thionyl chloride. Yield of the product was quantitative.

Step 2:

100 mg of ester derivative from step 1, 120 mg of HBTU, and 0.066 ml of DIEA were dissolved in 4.0 ml of dry DMF under argon and the reaction was stirred for 1 hour at RT. Then, 142 mg of (S)-2-((tert-butoxycarbonyl)amino)-5-((2,2,10,10-tetramethyl-4,8-dioxo-3,9-dioxa-5,7-diazaundecan-6-yl)amino)pentanoic acid was added under argon and the reaction mixture was stirred at RT for overnight. Once the reaction was completed, reaction contents were combined with 5.0 ml of ice cold water to precipitate the product out. Product was extracted into ethyl acetate. The organic layers were combined, dried and evaporated to obtain a product residue. This residue was then purified by chromatography to obtain the pure product. Yield of the product was 60%.

Step 3:

100 mg of the product from step 2 was dissolved in 5.0 ml of HPLC grade methanol and 0.5 ml of 2N NaOH was added to the reaction mixture. Reaction mixture was stirred at 50° C. for 8 hours. Once the reaction was completed, methanol was evaporated and the reaction mixture was combined with 0.5 ml of 1N HCl to neutralize the excess base. Subsequently, the resulting solution was extracted with ethyl acetate (20 ml). The organic layer was dried over sodium sulfate and concentrated using a rotary evaporator to obtain the product. Residue obtained in this way was purified by chromatography to obtain the desired product. Yield of the product was 75%.

Step 4.

50 mg of the product from step 3 was dissolved in 3.0 ml of 1:1 mixture of TFA:DCM and the resulting mixture was stirred 3 hours at RT. The reaction was monitored by TLC. Once the reaction was completed, the solvent was evaporated and the residue was dried under high vacuum. Product was purified using an amine bonded silica gel and a mixture of acetonitrile and methanol as an eluting solvent. Yield of the product was 70%.

### Solubility:

| | Measured | Predicted by ACD |
|---|---|---|
| Drug | (mg/ml) | PhysChem software v 12.0 |
| Gatifloxacin | 0.64 | 0.09 |
| GFX-ARG | 25.50 | 2.56 |

### Synthesis of 7-(4-(2-amino-4-methylpentanoyl)-3-methylpiperazin-1-yl)-1-cyclopropyl-6-fluoro-8-methoxy-4-oxo-1,4-dihydroquinoline-3-carboxylic acid [GFX-ATB (Leucine)]

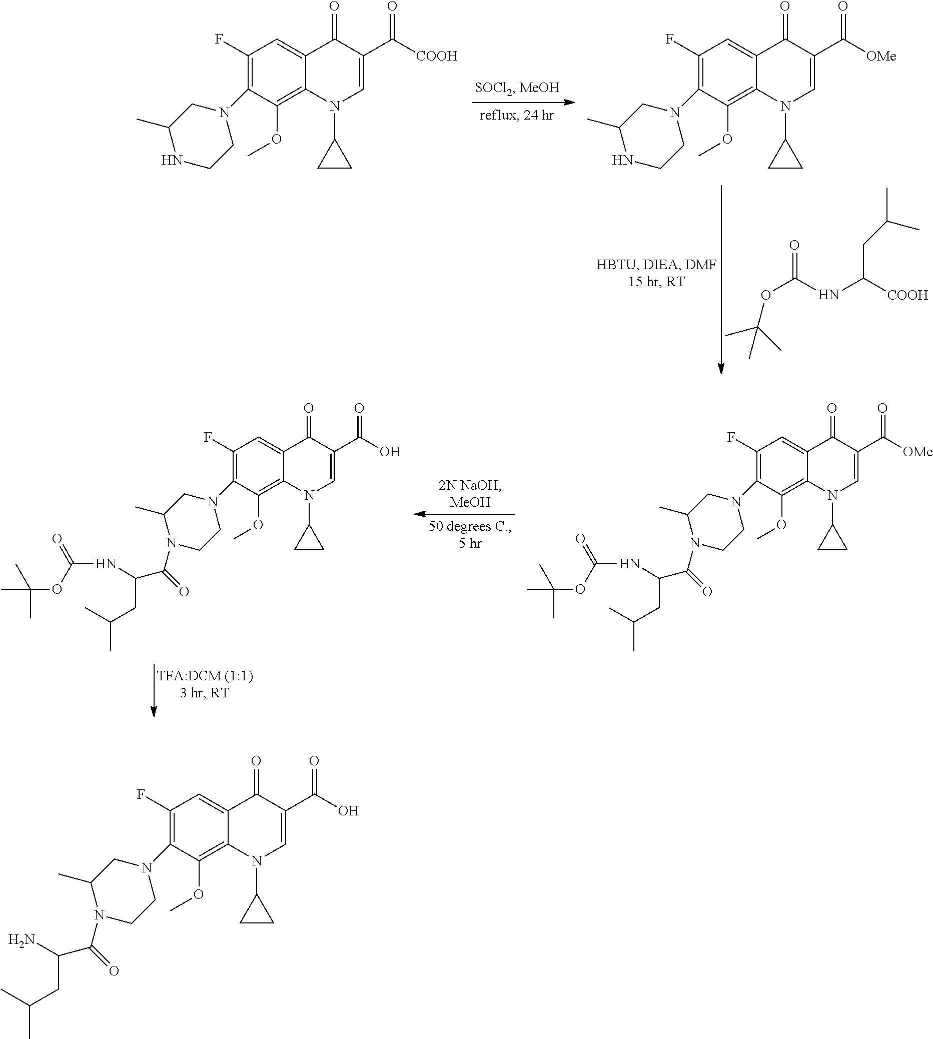 [SMILES: COc1c(N2CCNC(C)C2)c(F)cc2c(=O)c(C(=O)C(=O)O)cn(C3CC3)c12 ; COC(=O)c1cn(C2CC2)c2c(OC)c(N3CCNC(C)C3)c(F)cc2c1=O ; CC(C)CC(NC(=O)OC(C)(C)C)C(=O)O ; COc1c(N2CCN(C(=O)C(CC(C)C)NC(=O)OC(C)(C)C)C(C)C2)c(F)cc2c(=O)c(C(=O)O)cn(C3CC3)c12 ; COC(=O)c1cn(C2CC2)c2c(OC)c(N3CCN(C(=O)C(CC(C)C)NC(=O)OC(C)(C)C)C(C)C3)c(F)cc2c1=O ; COc1c(N2CCN(C(=O)C(N)CC(C)C)C(C)C2)c(F)cc2c(=O)c(C(=O)O)cn(C3CC3)c12]

Step 1:

250 mg of gatifloxacin was dissolved in 5.0 ml of HPLC grade methanol and the reaction flask was cooled to 0° C. Subsequently, 0.052 ml of thionyl chloride was added dropwise to the solution and the reaction contents were slowly brought to room temperature. Then the reaction was refluxed for 24 hours, cooled to RT, and the solvent was evaporated and the contents were dried under high vacuum and to remove the excess thionyl chloride. Yield of the crude product was quantitative.

Step 2.

150 mg of ester derivative from step 1, 267 mg of HBTU, and 0.135 ml of DIEA were dissolved in 4.0 ml of dry DMF under argon and the reaction was stirred for 1 hour at RT. Then, 109 mg of (R)-2-((tert-butoxycarbonyl)amino)-4-methylpentanoic acid was added and the reaction mixture was stirred at RT overnight. Once the reaction was completed, reaction mixture was combined with 5.0 ml of ice cold water. The mixture was extracted with ethyl acetate. The organic layer was dried and concentrated to obtain a residue. This residue was purified by chromatography to obtain the desired product. Yield of the product was 77%.

Step 3:

150 mg of the product from step 2 was dissolved in 5.0 ml of HPLC grade methanol and 0.7 ml of 2N NaOH was added to the solution mixture. Reaction mixture was stirred at 50° C. for 5 hours. Once the reaction was completed, methanol was evaporated and the reaction mixture was combined with 0.5 ml of 1N HCl. Subsequently, the product was extracted was ethyl acetate (20 ml). The organic layer was dried over sodium sulfate, filtered and concentrated to obtain a crude residue. The residue was purified by chromatography to obtain the desired product. Yield of the product was 80%.

Step 4:

60 mg of the product from step 3 was dissolved in 3.0 ml of 1:1 mixture of TFA:DCM. The resulting solution was stirred for 3 hours at RT. The solvent was evaporated and the residue was dried under high vacuum to obtain a crude product. Yield of the crude product was 90%.

### Synthesis of 1-cyclopropyl-7-(4-(2,6-diaminohexanoyl)-3-methylpiperazin-1-yl)-6-fluoro-8-methoxy-4-oxo-1,4-dihydroquinoline-3-carboxylic acid (GFX-LYS)

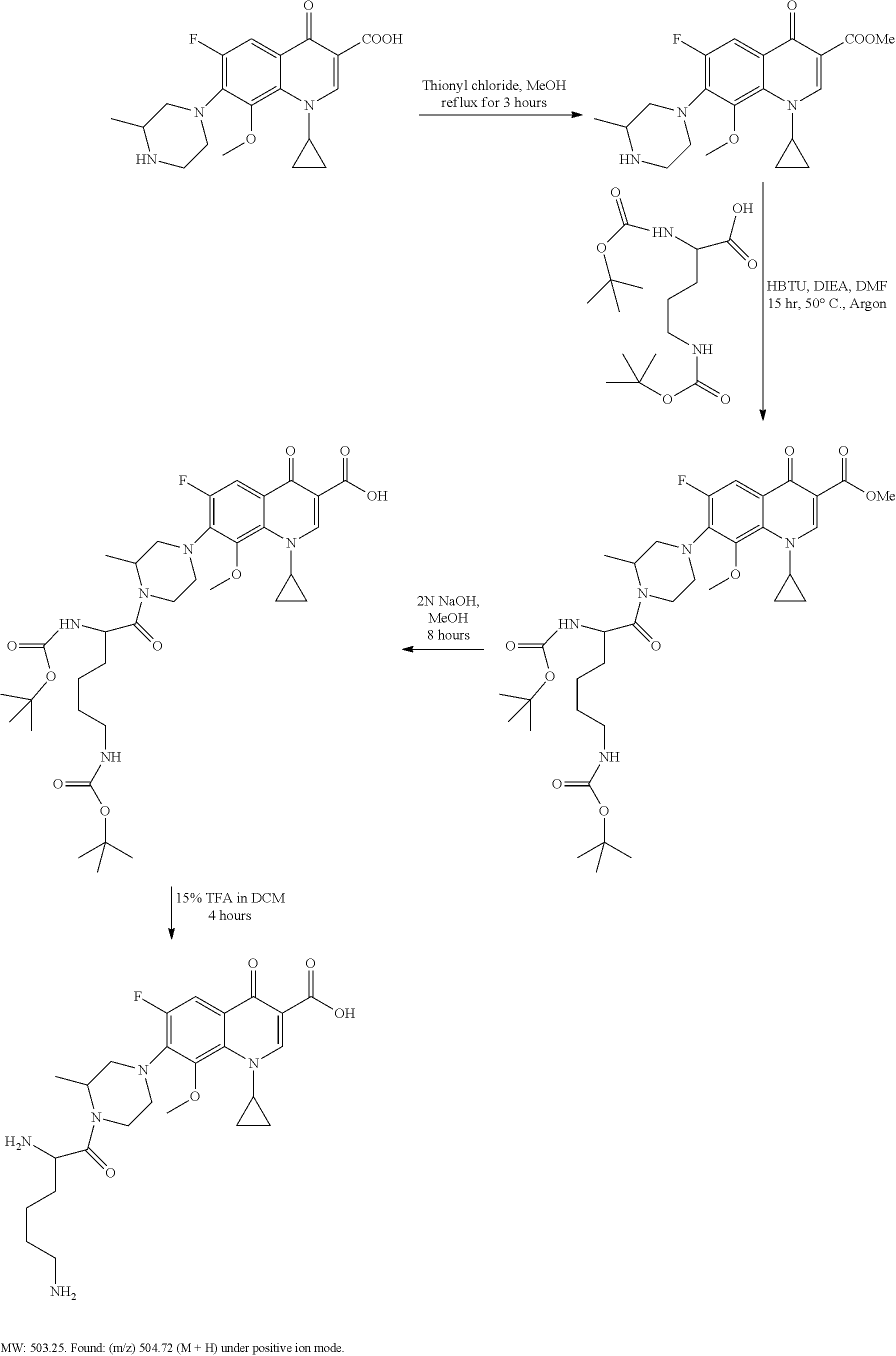

Step 1.

250 mg of gatifloxacin was dissolved in 5.0 ml of HPLC grade methanol and the reaction flask was cooled to 0° C. Subsequently, 0.052 ml of thionyl chloride was added dropwise to the solution mixture, and the reaction mixture was slowly brought to room temperature. The reaction mixture was refluxed for 24 hours, cooled to RT, concentrated, and the contents were dried under high vacuum. Yield of the crude product was quantitative.

Step 2:

194 mg of ester derivative from step 1, 282 mg of HBTU, and 0.132 ml of DIEA were dissolved in 4.0 ml of dry DMF under argon. The resulting mixture was stirred for 1 hour at RT. A suspension of 316 mg of 2,6-bis((tert-butoxycarbonyl)amino)hexanoic acid in 1.0 ml of dry DMF was added, and the resulting reaction mixture was stirred at 50° C. overnight. The reaction mixture was combined with 5.0 ml of ice cold water and extracted with ethyl acetate. The organic layer was dried and concentrated to obtain a crude residue. This residue was purified by chromatography to obtain the desired product. Yield of the product was 30%.

Step 3:

80 mg of the product from step 2 was dissolved in 5.0 ml of HPLC grade methanol and 0.5 ml of 2N NaOH was added. The reaction mixture was stirred at 50° C. for 5 hours. Methanol was evaporated and the resulting mixture was combined with 0.5 ml of 1N HCl. The resulting mixture was extracted with ethyl acetate (20 ml). The organic layer was dried over sodium sulfate, filtered, concentrated and purified by chromatography to obtain the desired product. Yield of the product was 65%.

Step 4:

40 mg of the product from step 3 was dissolved in 3.0 ml of 15% solution of TFA in DCM. The resulting mixture was stirred for 4 hours at RT. The solvent was evaporated and the residue was dried under high vacuum. The desired product was obtained by chromatography using an amine bonded silica gel a mixture of acetonitrile and methanol as eluting solvent. Yield of the product was 80%.

### Solubility:

| | Measured | Predicted by ACD |
|---|---|---|
| Drug | (mg/ml) | PhysChem software v 12.0 |
| Gatifloxacin | 0.64 | 0.09 |
| GFX-LYS | 48.00 | 0.15 |

### Synthesis of 4-(1-cyclopropyl-6-fluoro-8-methoxy-7-(3-methylpiperazin-1-yl)-4-oxo-1,4-dihydroquinoline-3-carboxamido)butanoic acid (GFX-MCT)

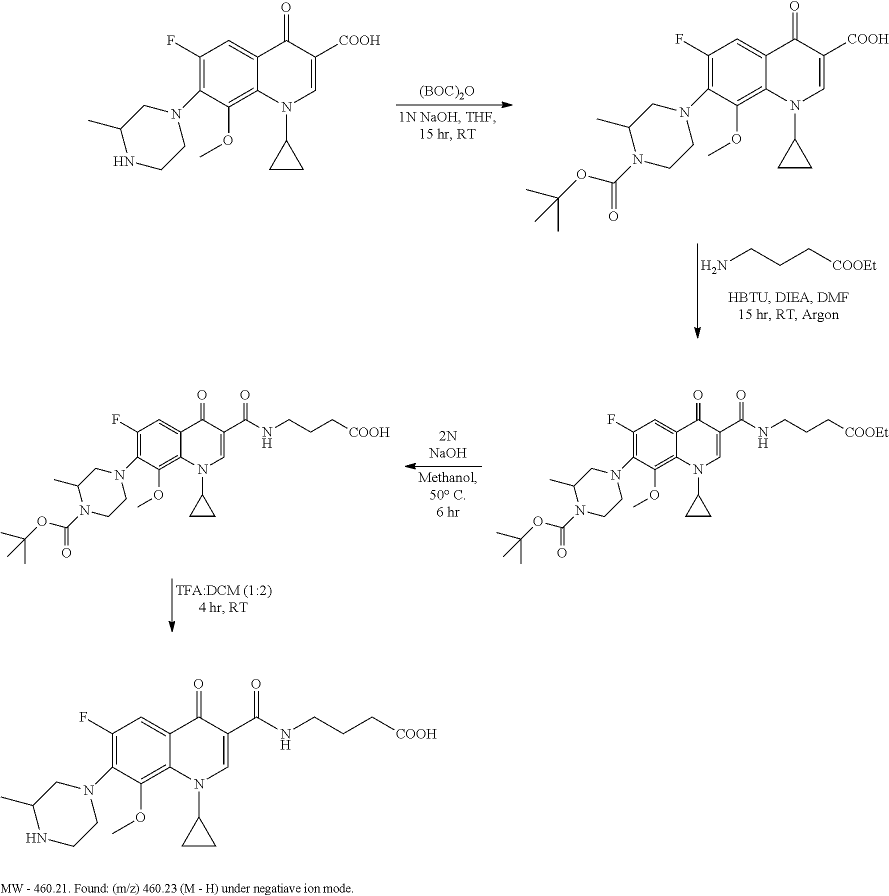

Step 1.

200 mg of gatifloxacin sesquihydrate was dissolved in dry tetrahydrofuran solvent (5.0 ml) and 0.5 ml of 1N NaOH was added. Subsequently, 124 mg of boc-anhydride was added, and the reaction mixture was stirred under argon overnight at room temperature. The solvent was evaporated and the residue was diluted with aqueous saturated ammonium chloride solution. The resulting solution was extracted with ethyl acetate (2×20 ml). The organic layers were combined, dried over sodium sulfate and concentrated to obtain a crude product. Yield of the crude product was 68%.

Step 2.

100 mg of boc-protected gatifloxacin (from step 1), 120 mg of HBTU, and 0.069 ml of DIEA were dissolved in dry DMF under argon and the mixture was stirred for 1 hour at RT. Then, 39 mg of ethyl 4-aminobutanoate was added and stirred at RT overnight. The resulting reaction mixture was combined with 5.0 ml of ice cold water, extracted with ethyl acetate. The organic layer was dried, concentrated and purified by chromatography to obtain the desired product. Yield of the desired product was 85%.

Step 3.

90 mg of ester derivative (from step 2) was dissolved in 5.0 ml of HPLC grade methanol and 0.7 ml of 2N NaOH was added. The resulting mixture was stirred at room temperature overnight. The reaction was warmed to 50° C. and stirred for additional 4 hours. Methanol was evaporated and the resulting mixture was combined with 1N HCl. The aqueous solution was extracted with ethyl acetate (20 ml). The organic layer was dried over sodium sulfate, filtered and concentrated to obtain the desired product. Yield of the desired product was quantitative.

Step 4.

50 mg of acid derivative (from step 3) was dissolved in 3.0 ml of 1:1 mixture of TFA:DCM and stirred 3 hours at RT. The reaction mixture was concentrated, and the residue was dried under high vacuum to obtain the desired product in 85% yield.

### Synthesis of 1-cyclopropyl-N-(3-(dimethylamino)propyl)-6-fluoro-8-methoxy-7-(3-methylpiperazin-1-yl)-4-oxo-1,4-dihydroquinoline-3-carboxamide (GFX-OCT)

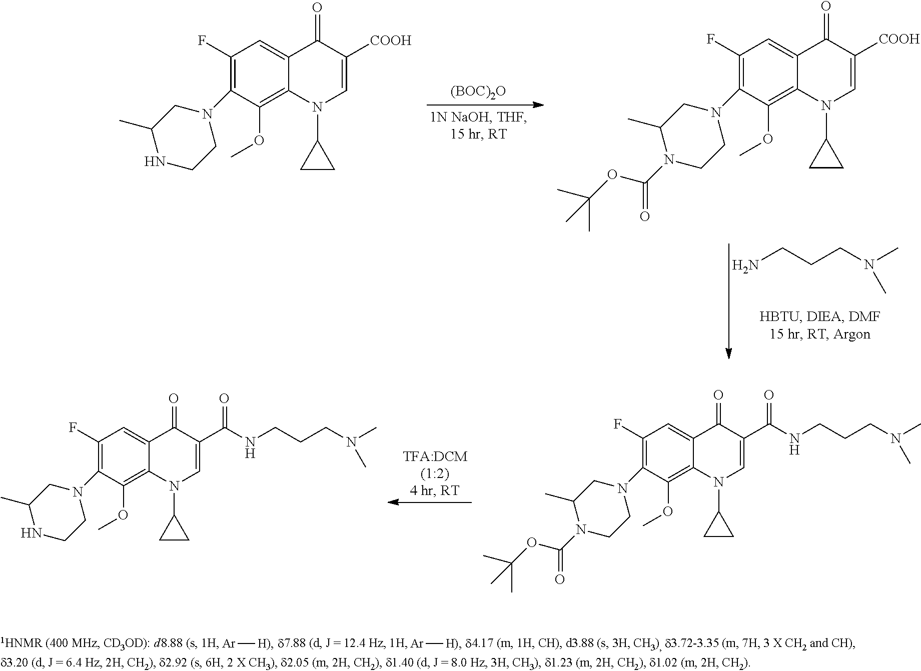

Step 1.

200 mg of gatifloxacin sesquihydrate was dissolved in dry tetrahydrofuran solvent (5.0 ml) and 0.5 ml of 1N NaOH was added. Subsequently, 124 mg of boc-anhydride was added and the resulting mixture was stirred under argon overnight at room temperature. The reaction mixture was concentrated, and the residue was diluted with saturated aqueous ammonium chloride solution. The aqueous mixture was extracted with ethyl acetate (2×20 ml). The organic layers were combined, dried over sodium sulfate, filtered and concentrated to obtain the desired product in 68% crude yield.

Step 2.

80 mg of boc-protected gatifloxacin (from step 1) and 76 mg of HBTU were dissolved in dry DMF under argon and the mixture was stirred for 1 hour at RT. Then, 0.027 ml of N¹,N¹-dimethylpropane-1,3-diamine was added and the resulting mixture was stirred at RT overnight. The reaction mixture was concentrated and purified by chromatography to obtain the desired product in 45% yield.

Step 3.

40 mg of the product from step 2 was dissolved in 3.0 ml of 1:2 mixture of TFA:DCM and stirred at RT for 4 hours. The reaction mixture was concentrated to obtain the desired product in 75% crude yield.

### Results

Patient Demographics and H & E Staining of Human Eyes:

Paraffin embedded 5-7 μm thick sections of whole eyes were obtained from patients. Demographic information of patient data are summarized in Example 2 above. Whole eye sections were stained with hematoxylin and eosin (H&E) for anatomical assessment of ocular structures. H&E stain image of cornea showed (data not shown) intact 3-4 layers tight epithelium followed by thick stroma with sparse fibroblasts and single layer of endothelial cells. Conjunctival H&E stained showed multilayer columnar epithelium followed by stroma with larger number of fibroblast and globet cells. H&E stain of ciliary body showed the single layer of inner non-pigmented epithelial cell followed by outer pigmented epithelium and ciliary muscles which attach it to the sclera. Histological section of sclera-choroid-retina (SCR) showed all 8 distinguished layers of retina, single layer of retinal pigmented epithelium (RPE), choroid and sclera.

Localization of PEPT-1 and PEPT-2 in Human Ocular Tissues:

Four eyes from four donors (see Example 2) were examined for PEPT transporter expression. Of these two PEPT transporters, PEPT-1 staining was less abundant than PEPT-2 staining in all ocular tissues (data not shown). PEPT-1 showed very light immunolabeling around the margins of the basal cells of corneal and conjunctival epithelium. Nonpigmented ciliary epithelium showed more intense immunolabeling than cornea and conjunctiva. With regards to SCR, for PEPT-1, light staining was localized to inner nuclear and ganglion cell layer of retina, RPE, and smooth muscles of choroidal blood vessels. PEPT-2 showed very intense staining in all ocular tissues assessed. In cornea and conjunctiva, PEPT-2 immunolabeling was observed only in epithelial layers with uniform distribution throughout the epithelial layers. In ciliary body, PEPT-2 immunolabeling was only observed in non-pigmented ciliary epithelium. For SCR, PEPT-2 showed very strong labeling in outer segment of rod cells of retina. PEPT-2 labeling was also seen in ganglion cell layer of retina, RPE, and smooth muscles of choroidal blood vessels.

Localization of ATB⁰⁺ in Human Ocular Tissues:

Immunohistochemical labeling of ATB⁰⁺ in human ocular tissues showed the expression of ATB⁰⁺ in cornea, conjunctiva, ciliary body, retina and RPE with staining confounding near the nucleus. As slides were counter stained with hematoxylin, and ATB⁰⁺ labeling was visualized using Poly-AP red, co-localization of red and blue signal showed a reddish brown color instead of red color. In cornea and conjunctiva, ATB⁰⁺ labeling was concentrated around the basal cells of epithelium. Fibroblast cells in corneal and conjunctival stroma also showed the labeling with ATB⁰⁺. Non-pigmented ciliary epithelium showed bright ATB⁰⁺ staining than all other tissues. For SCR, ATB⁰⁺ labeling was observed in inner and outer nuclear layer, ganglion cell layer as well as in RPE.

Immunohistochemical Localization of OCT-1 and OCT-2 in Human Ocular Tissues:

Of these two organic cation transporters assessed, immunostaining in retina was observed only in OCT-1. (Data not shown). OCT-1 showed the brighter staining in corneal epithelium than conjunctival epithelium. Furthermore, OCT-1 labeling was also observed in corneal endothelium. In SCR, OCT-1 labeling appeared to be localized to inner segment of photoreceptor cell and RPE layer. Light staining with OCT-1 was also observed in smooth muscles of choroidal blood vessels.

Non-pigmented and pigmented ciliary epithelium and choroid-retina were substantially devoid of OCT-2 labeling. In corneal and conjunctival epithelium, OCT-2 expression was localized more towards outer layer of epithelium. In conjunctiva, OCT-2 labeling was also observed in smooth muscles of conjunctival blood vessels.

Immunohistochemical Localization of MCT-1 and MCT-2 in Human Ocular Tissues:

Immunohistochemical analysis of expression of monocarboxylate transporter (MCT) 1 in human ocular tissue showed the immunolabeling in the basal cells of epithelium near the border of epithelium and stroma in cornea and conjunctiva and endothelium of cornea. Non-pigmented ciliary epithelium showed a strong labeling with MCT-1. For SCR, MCT-1 labeling was observed in inner limiting membrane, outer segment of photoreceptor cells, and RPE cell layer. In case of MCT-3, immunohistochemical labeling was seen in RPE layer. No significant MCT-3 staining was observed in cornea, conjunctiva and ciliary body.

Transport of Transporter Substrate Cassette Across Human Sclera-Choroid-RPE (SCRPE):

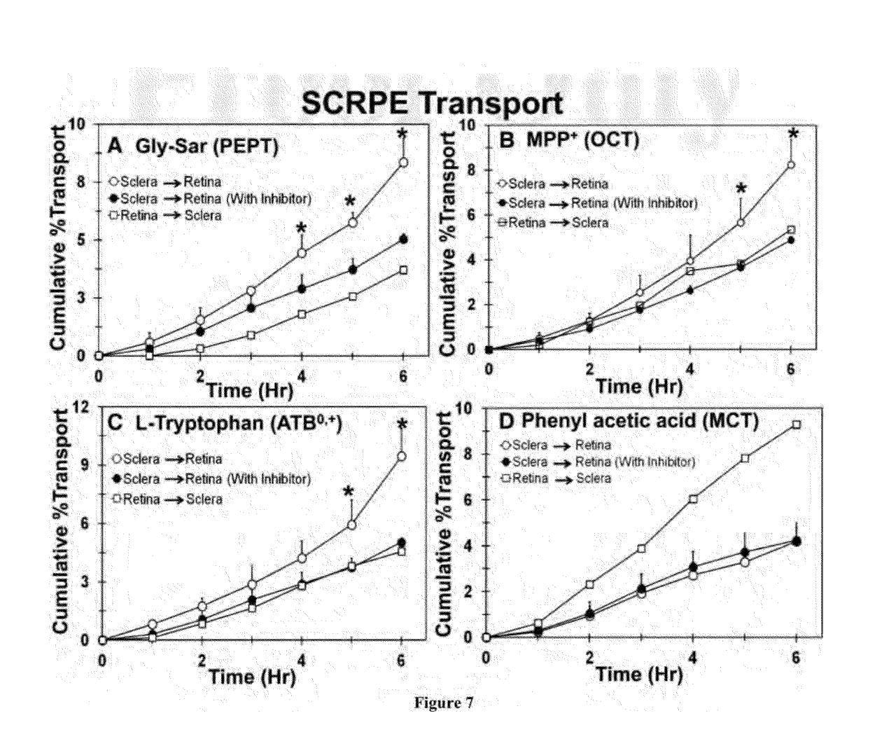

Transport of transporter specific substrates was carried out across human SCRPE for functional evaluation of activity of transporter in SCRPE barriers. Cassette dosing approach was used to reduce the tissue usage and increase the throughput. Effect of directionality was evaluated to determine whether a particular transport was contributing in influx of drug to retina or efflux from retina. As shown in FIGS. 7A-C, transport of Gly-Sar, MPP⁺ and L-tryptophan from sclera to retinal direction was significantly higher than retinal to sclera direction indicating that these transporters were acting as influx transporter in retinal drug delivery. In contrast, for phenyl acetic acid (MCT substrate), retina to sclera transport was significantly higher than sclera to retina transport, thereby indicating it was playing the role in efflux of molecules from retina to choroid (FIG. 7D).

Furthermore, sclera to retina direction transport experiments were carried out in the presence and the absence of a specific transporter inhibitor to evaluate the contribution of the active transporter mediated transport in total transport across SCRPE. As shown in FIGS. 7A-C, sclera to retinal direction transport of Gly-Sar, MPP⁺ and L-tryptophan were significantly inhibited in the presence of a transporter inhibitor indicating that the transporter mediated transport across human SCRPE. In case of phenyl acetic acid (PHA), there was no significant effect of inhibitor on sclera to retinal direction transport (FIG. 7D).

Transport of Transporter Substrate Cassette Across Human Cornea:

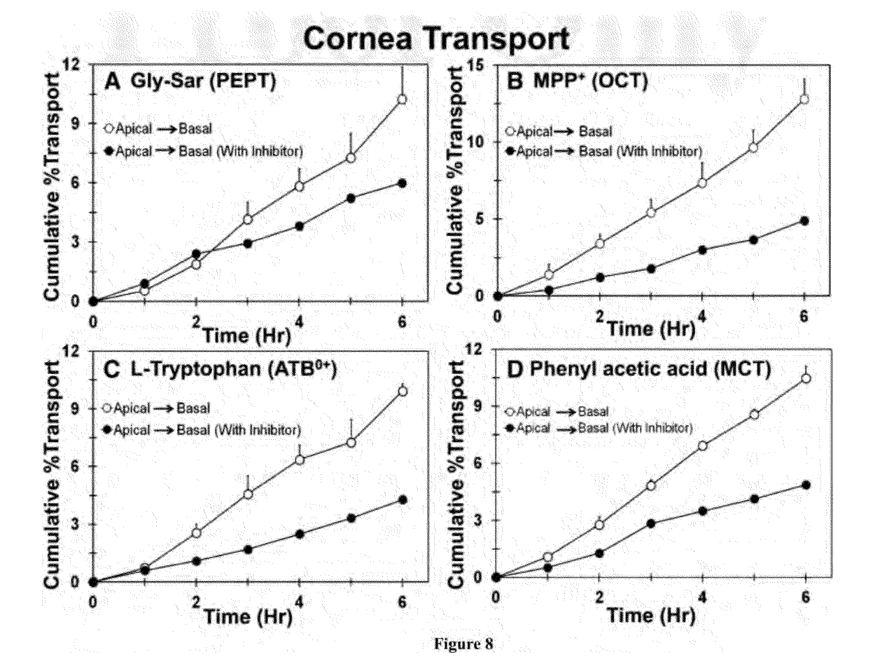

Transport of transporter substrate cassettes across cornea was evaluated in the presence and the absence of a specific inhibitor. As shown in FIGS. 8A-D, apical to basal direction transport of all four transporter substrates were significantly inhibited in the presence of a transporter inhibitor. Immunohistochemical analysis showed PEPT-2 expression was abundant in cornea; however, it appears the inhibitor concentration used for competitive inhibition was not sufficient to inhibit the maximum transport of Gly-Sar. Thus, the difference in the transport was not very significant in the presence and the absence of an inhibitor.

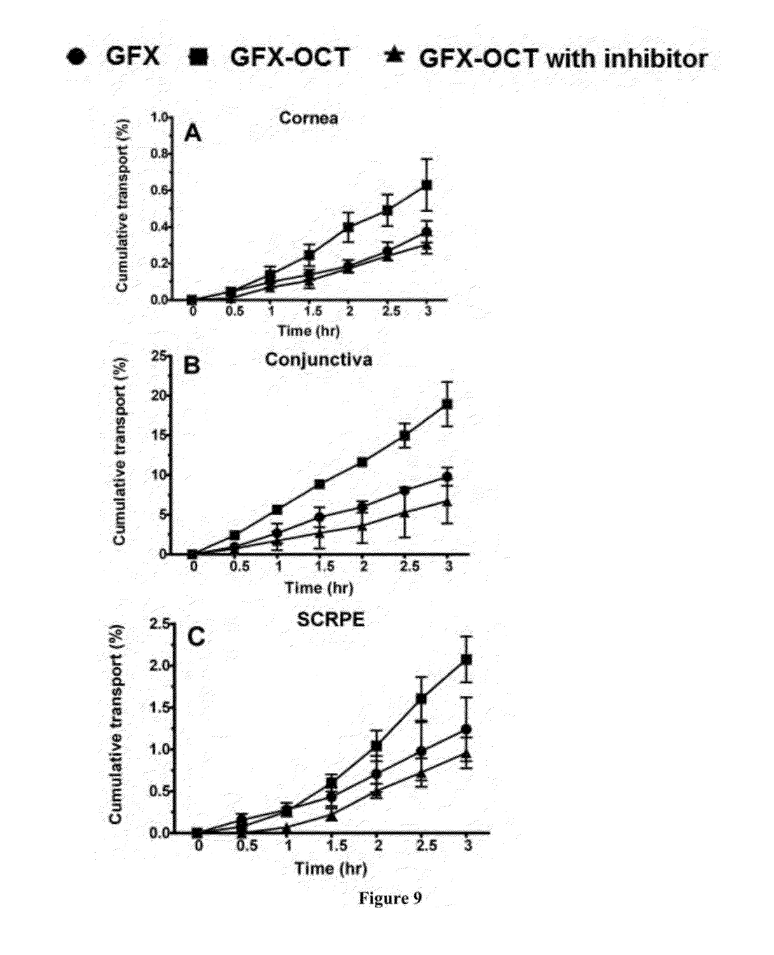

In vitro delivery of GFX prodrugs across rabbit cornea, conjunctiva, and SCRPE: As shown in FIG. 9, cumulative % transport of GFX-OCT prodrug was significantly higher than compared to GFX across cornea, conjunctiva, and SCRPE tissues. Moreover, transport of GFX-OCT prodrug was significantly inhibited across all tissues in presence of MPP+, a competitive inhibitor. Thus, the transport of GFX-OCT prodrug was mediated by OCT.

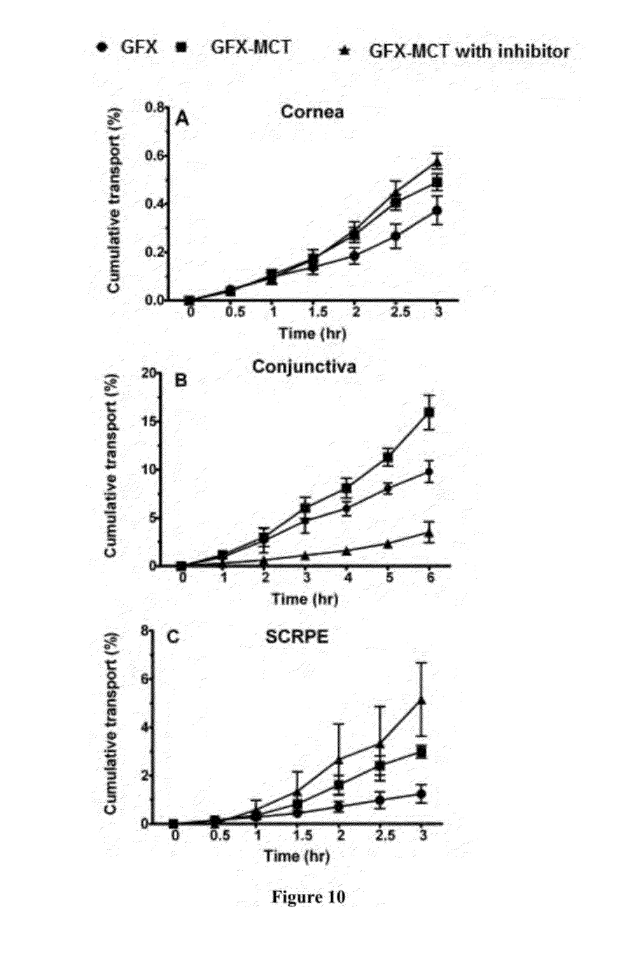

As shown in FIG. 10, cumulative % transport of GFX-MCT prodrug was significantly higher than GFX across cornea, conjunctiva, and SCRPE. Transport of GFX-MCT was significantly inhibited by nicotinic acid (competitive inhibitor of MCT) across conjunctiva.

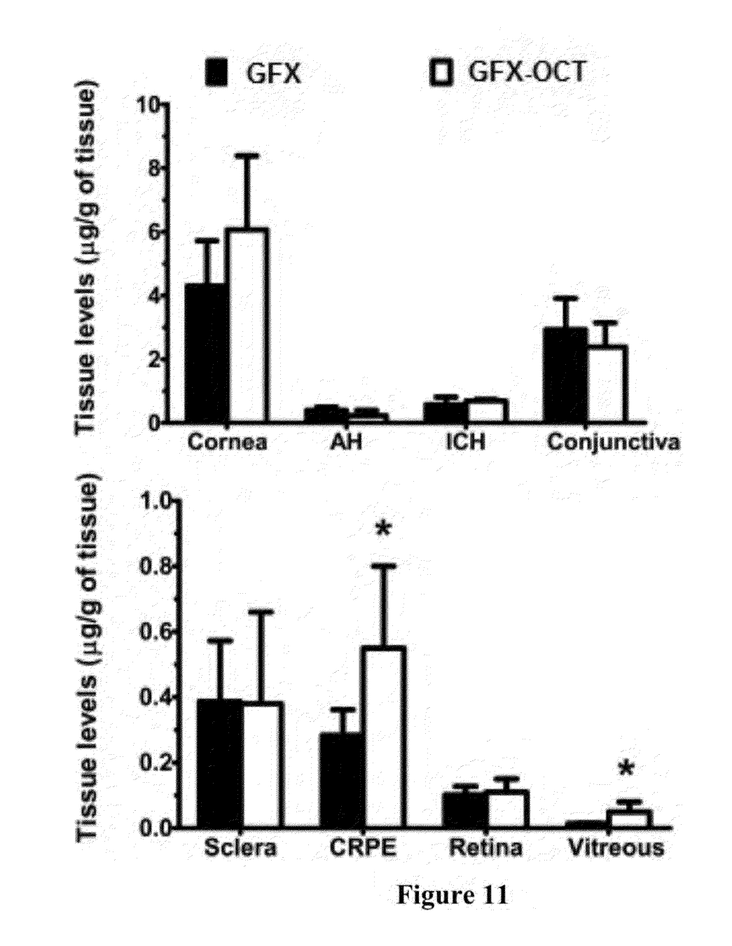

Comparison of in vivo topical delivery of GFX-OCT prodrug with GFX: To evaluate the influence of the GFX-OCT prodrug on intraocular delivery, an in vivo ocular tissue distribution study was conducted in pigmented rabbits after topical dosing and compared with topical administration of GFX alone. Comparison of in vivo ocular tissue distribution of GFX and the GFX-OCT ion pair after topical application is shown in FIG. 11. As can be seen in FIG. 11, GFX-OCT prodrug levels in posterior tissues such as vitreous humor (3.6-fold) and CRPE (1.95-fold) is significantly higher than GFX. However, the levels of GFX-OCT prodrug were significantly higher across anterior tissues including cornea, conjunctiva, aqueous humor, sclera, and ICB.

### Discussion

The present inventors have investigated the functional characterization of influx drug transporters in human ocular tissues. In particular, the expression and functional activity of PEPT, OCT, ATB⁰⁺, and MCT transporters were characterized using immunohistochemistry and in vitro transport studies in human ocular tissues. Immunohistochemical analysis of transporter expression in whole human eye sections showed the differential expression of drug transporters in various ocular tissues. In some instances, multiple isoforms of the same transporter was localized in different human ocular tissues. Out of the four transporters assessed in the sclera-choroid-retina, PEPT, OCT, and ATB⁰⁺ were determined to be influx transporters and MCT was an efflux transporter. Directional transport from sclera-to-retina across SCRPE via PEPT, OCT, and ATB⁰⁺ was significantly inhibited in the presence of a transporter specific inhibitor. In case of cornea, all four transporters were influx transporters, with the transport being inhibited by the presence of a transporter inhibitor.

The present inventors have discovered that there was a strong expression of PEPT-2 transporter proteins in cornea, conjunctiva, ciliary body, choroid, and retina of the human eye. For PEPT-1, the present inventors have observed a light staining in corneal and conjunctival epithelia, inner nuclear layer of the retina, and the RPE. As a solute carrier transporter in transport of dipeptide across biological barriers, ubiquitous distribution of this transporter in ocular tissue is believed to be necessary for physiological function. Previous studies with gene expression analysis of PEPT transporters in human ocular tissues showed a strong expression of PEPT-2 and a weak expression of PEPT-1. These studies also showed the absence of expression of PEPT-1 transporter in human choroid-retina. In contrast, the present inventors have observed a light expression of PEPT-1 transporter in inner nuclear layer of the retina, RPE, and choroidal smooth muscles. The present inventors have also observed a strong expression of PEPT-2 and a light expression of PEPT-1 transporters in epithelial cells of bulbar conjunctiva.

Due to broader substrate specificity and relatively ubiquitous distribution of ATB⁰⁺, ATB⁰⁺ was selected for characterization in human ocular tissues. Previous studies also showed the significant implication of ATB⁰⁺ in ocular drug delivery. In the current study, the present inventors observed the expression ATB⁰⁺ in corneal and conjunctival epithelia, inner and outer nuclear layers and ganglion cell layer of retina, RPE, and ciliary epithelium. Expression of ATB⁰⁺ was abundant in RPE and non-pigmented ciliary epithelium, when compared to other ocular barriers. Without being bound by any theory, it is believed that relatively ubiquitous distribution of ATB⁰⁺ in all nuclear layers of retina is due to the high need for amino acids such as glycine for neurotransmission as well as protein synthesis in the retina.

Another transporter explored by the present inventors in human ocular tissues was organic cation transporter (OCT). The m-RNA analysis showed abundant expression of this transporter in human ocular tissues. The present inventors have discovered that ophthalmic cationic drugs such as brimonidine, timolol, betaxolol can be actively transported by OCT. For OCT, immunohistochemistry analysis was performed for two isoforms, OCT-1 and OCT-2. Although some studies have shown the abundant gene expression of carnitine organic cation transporters (OCTN) in human ocular tissues, immunohistochemical analysis for the OCTN transporters was not performed because of availability of literature reports on immunohistochemical characterization of OCTN in human ocular tissues. The present inventors have also discovered that there was a significant expression of OCT-1 transporters in human cornea, conjunctiva, and non-pigmented ciliary epithelium, inner segment of rod cells, RPE layer and smooth muscles of choroidal blood vessels. For OCT-2 transporters, immunolabeling was localized to corneal and conjunctival epithelia and corneal endothelium. Expression of OCT-1 in cornea, conjunctiva and RPE and OCT-2 in cornea and conjunctiva can be utilized for transporter guided intraocular delivery of cationic drug molecules.

Another transporter the present inventors have characterized in human ocular tissues was monocarboxylate transporters (MCT). Gene expression analysis of MCT transporters in rat ocular tissues by others showed that MCT-1 and MCT-3 are the most abundant isoforms of MCT. Accordingly, these isoforms were selected for immunohistochemical analysis. For MCT-1, immunohistochemical labeling was observed in photoreceptors cells, inner retinal layer, RPE, iris ciliary body, corneal and conjunctival epithelia, and corneal endothelium. For MCT-3, the immunostaining was observed in the RPE layer but no noticeable staining was observed in any other ocular tissues. Previous reports with human and rat tissues have also shown that immunolocalization of MCT-3 was observed in RPE layer but not in any other ocular tissue. In addition, a previous immunolabeling and western blot analysis of human RPE showed that the MCT-1 expression was higher than the MCT-3 expression.

Currently, it appears that no report is available showing the functional characterization of these four classes of transporters in human ocular barriers. To confirm the functional activity and directionality of these four classes of transporters, in vitro transport studies were carried out across human SCRPE and cornea. Cassette dosing approach was used to increase the throughput. Similar to other high throughput assays, cassette dosing method is associated with its own disadvantages. For example, in a given cassette the cross reactivity of transporter substrates and inhibitors with more than one transporter cannot be completely ruled out. However, extra precaution was taken during the selection of transporter substrates and inhibitors to avoid cross reactivity with other transporters. See Table below.

TABLE
List of transporter, specific substrates and inhibitors
for particular transporter, and inhibition mechanism.

| Trans- | Specific | Specific | |
|---|---|---|---|
| porter | Substrate | Inhibitor | Inhibition Mechanism |
| PEPT | Gly-Sar | H-Pro-Phe-OH | Competitive Inhibition |
| OCT | MPP+ | Metformin | Competitive Inhibition |
| ATB⁰⁺ | L-Tryptophan | α-Methyl | Specific Inhibition |
| | | Tryptophan | |
| MCT | Phenyl Acetic | Nicotinic acid | Competitive Inhibition |
| | Acid | | |

Specific substrates and inhibitors were carefully selected based on the unique structural requirements of individual transporters. Dipeptide Gly-Sar is a well known substrate for PEPT transporters. Characterization of several hundred substrates/inhibitors of PEPT transporters using Gly-Sar as control have previously been reported. Gly-Sar is selectively transported by PEPT transporters and it is expected to not have any significant cross reactivity with ATB⁰⁺, MCT, and OCT transporters. ATB⁰⁺ does exhibit broad substrate selectivity towards all amino acids, however, it cannot transport the dipeptide (Gly-Sar) due to the requirement that α-COOH group of the amino acid be either free acid or esterified. Pro-Phe has higher affinity than Gly-Sar towards PEPT1 and PEPT2. Therefore, Pro-Phe strongly inhibits the transport of Gly-Sar.

MPP⁺ is the common substrate of OCT transporters. In fact, MPP⁺ is transported by all forms of OCT including OCT1, OCT2, and OCT3 as well as OCNT transporters. MPP⁺ is a highly selective substrate for OCT and is not transported by PEPT, MCT, and ATB⁰⁺. Metformin inhibits the transport of MPP⁺ by OCT1, OCT2, and OCT3 transporters. Phenformin and cimetidine are more potent inhibitors than metformin; however, metformin was used in experiments because it does not have any significant interaction with efflux transporters such as MDR and MRP.

ATB⁰⁺ is known to have broad substrate selectivity, and it can transport 18 of the proteinogenic amino acids with L-Tryptophan having higher binding affinities than other amino acids. Phenyl acetic acid was used as an inhibitor in experiments for ease of detection using LC-MS/MS. Phenyl acetic acid and nicotinic acid are transported by MCT transporters and do not have any significant cross reactivity with ATB⁰⁺ and PEPT transporters.

Transport of Gly-Sar, L-tryptophan, and MPP⁺ across human SCRPE and cornea showed the involvement of influx transporters. Sclera to retina transport for Gly-Sar, L-tryptophan, and MPP⁺ was 1.6- to 2.0-fold higher than retina to sclera transport (FIG. 7) and it was inhibited by 1.6 to 1.9-fold in the presence of specific inhibitors (FIG. 7), indicating transporter mediated influx of these molecules across human SCRPE. Further, the corneal transport of all three molecules was significantly inhibited in the presence of inhibitors. In vitro transport study of Gly-Sar across albino rabbit SCRPE showed the involvement of PEPT transporter and that Gly-Sar transport was significantly inhibited (1) in the presence other PEPT transporter substrates and inhibitors, and (2) a reduction in temperature form 37° C. to 4° C. Due to abundant expression pattern in human ocular tissue and wide substrate selectivity, PEPT transporters can be used for transporter mediated ocular drug delivery.

Although expression of OCT isoforms tested in current study was not as abundant as PEPT transporters, the cumulative % transport of Gly-Sar and MPP⁺ across SCRPE and cornea was comparable. With immunohistochemical analysis, the expression of OCT-1 and OCT-2 isoforms was determined. MPP⁺ as a substrate for OCT transporter has broad selectivity and interacts with both OCT as well as OCTN transporters. MPP⁺ transport across human SCRPE and cornea was 1.7 and 2.6-fold lower, respectively, in the presence of OCT inhibitor metformin. Abundant expression in human ocular barriers, wide substrate selectivity and cross reactivity of substrates between different isoforms of OCT and OCTN transporter render these transporters well suited for use in ocular drug delivery. In addition, many ophthalmic drugs are cationic molecules and their passive permeability at physiological pH is limited by the ionic state. Utilization of organic cation transporters (OCT and OCTN) in ocular delivery of poorly permeable cationic drugs can overcome the delivery problem.

MCT transporter has been shown to act as influx and efflux transporter for monocarboxylic acid compounds such as lactate, pyruvate and ketone bodies. Surprisingly and unexpectedly, the present inventors have discovered that MCTs act as influx transporters in cornea and as efflux transporters in SCRPE. Retina is highly metabolically active compared to several other tissues and has shown to produce large amounts of lactic acid by aerobic metabolism of glucose. MCT1 and MCT3 in retina and RPE act as efflux transporters to remove lactate from subretinal space to the choroidal circulation and to maintain cellular homostasis. MCT acts as an influx transporter in cornea and conjunctiva to reabsorb lactate from tear fluid, where lactate is present at a relatively very high concentration (1 to 5 mM). Same isoform of MCT can act as influx or efflux transporters in hypothalamic glial cells depending upon the glucose and lactate concentration available in the media. Bidirectional transport ability of MCT transporter can be used in transporter mediated delivery of monocarboxylic acid drug molecules across ocular barriers.

Transporter mediated delivery of the GFX prodrugs was evaluated. Some of the key findings of the present disclosure are that: (1) GFX-OCT prodrug transport is mediated by OCT. (2) Cumulative % transport of GFX-OCT prodrug is significantly higher than GFX across all rabbit tissues including cornea, conjunctiva, and SCRPE. (3) Cumulative % transport of GFX-MCT prodrug is significantly higher than GFX across rabbit cornea, conjunctiva, and SCRPE. (4) However, cumulative % transport of GFX-MCT was only inhibited by nicotinic acid across conjunctiva, but not across SCRPE. In fact we also saw similar results with transport of phenyl acetic across SCRPE (sclera to retina direction) as shown in FIG. 7. To date there are no reports on ocular drugs targeting OCT and MCT transporters.

To demonstrate the effectiveness of the GFX-OCT prodrug for topical ocular drug delivery, in vivo ocular delivery experiments were performed in normal pigmented rabbits using clear aqueous solution of the GFX-OCT prodrug and the results were compared with GFX (i.e., GFX alone). Drug levels were compared at the end of 1 hr because the present inventors have discovered the peak drug concentrations in posterior ocular tissues after topical application were at around 1 hr post dosing. Since the present inventors have discovered that GFX-OCT is transported at least in part by OCT transporters across ocular barriers, GFX delivery to the intraocular tissues was expected to be higher for the GFX-OCT prodrug than GFX alone. As expected, in vivo ocular tissue distribution study showed that the GFX-OCT had higher concentrations of GFX in all posterior tissues such as vitreous humor and CRPE than GFX. However, in remaining all tissues, levels of GFX-OCT prodrug was not significantly higher than GFX alone. Inventors believe that this is partly because of the different Tₘₐₓ of GFX is different for different ocular tissues following a single eye drop study in pigmented rabbits. For example, reported Tₘₐₓ for cornea, and conjunctiva is 0.083 h, 0.33 h for aqueous humor, whereas the tissues were collected at 1 h in this study.

### Conclusion

The present inventors have discovered the immunochemical and functional evidence for drug transporters (e.g., PEPT, ATB⁰⁺, OCT, and MCT) in human ocular tissue. The present inventors have also observed that PEPT, ATB⁰⁺, and OCT are influx transporters. These transporters were relatively ubiquitously distributed in ocular barriers. These transporters also have wide substrate selectivity, and can be used in a wide variety of transporter mediated intraocular drug delivery. MCT transporter acts as an influx transporter in cornea and as an efflux transporter in SCRPE and can be used for delivery of monocarboxylate drug molecules.

Topical drug delivery to the intraocular tissues is restricted by poor permeability across ocular barriers. Utilization of uptake drug transporters present in ocular barriers is helpful in improving uptake of poorly permeable hydrophilic drugs. In this study, using GFX as a model amphiphillic drug with antibacterial effects in the treatment of ocular infectious disease, the present inventors have shown that the intraocular delivery of GFX can be significantly enhanced in vitro and in vivo through formation of a GFX prodrug targeting transporters present in ocular barriers. Results from this study provide new insights into the underlying mechanisms for enhanced delivery with transporter targeted prodrug for poorly permeable drugs. Utilization of this approach with proper selection of prodrug moieties for transporter guided drug delivery in topical drug delivery to the eye tissues can be used effectively to treat various ocular diseases and clinical conditions associated with ocular tissues.

### Example 3

While age related macular degeneration (AMD) and diabetic retinopathy are leading causes of blindness in adults, retinopathy of prematurity (ROP) is a leading causes of blindness in infants. Neovascularization of retina and/or choroid is the hallmark of these diseases, with tissue hypoxia being a key cause. Expression of several angiogenic and anti-angiogenic factors are oxygen dependent and controlled by hypoxia inducible factor. Hypoxia stimulates the release of hypoxia induced cytokines including vascular endothelial growth factor (VEGF) that is responsible for retinal neovascularization. Capillary loss in retina or impairment of choroidal blood vessels can result in hypoxia development. Development of hypoxia in choroid/retina stimulates VEGF release, thereby causing choroidal/retinal angiogenesis. In animal models of retinal neovascularization, hypoxia induced VEGF levels correlate with neovascularization.

Retina is a metabolically active tissue and needs large amounts of nutrients to produce metabolic energy for photo-transduction and neuro-transduction. Glycine, an amino acid important for the synthesis of glutathione and creatine, plays a significant role in neurotransmission in retina. The glycine concentration in neural retina is 5-fold higher than in plasma and it accumulates in retina through highly concentrative Na⁺ and Cl⁻ dependent glycine transporters. Similar to the brain, retina is protected by inner and outer blood retinal barriers (BRB) to maintain its controlled environment. The BRB comprising retinal capillary endothelial cells (inner BRB) and retinal pigmented epithelial cells (RPE; outer BRB), restricts nonspecific transport of solutes from the blood to the retina. Metabolic substrates such as glucose and amino acids are hydrophilic and their passive permeability is restricted by BRB. BRB expresses various nutrient and neurotransmitter transporters to allow their selective entry into the retina. Expressions of these transporters in BRB may be altered during hypoxia.

Hypoxia can influence the expression and functional activity of solute carrier transporters in biological tissues, thereby contributing to the disease pathology. Hypoxia elevates retinal levels of glucose, a casual factor for the development of diabetic retinopathy. Hypoxia results in increased expression of glucose transporters that are responsible for increased glucose uptake. In pregnant women, placental hypoxia is considered as an underlying cause for fetal growth restriction, preeclampsia, and diabetes. Various studies have reported altered expression of solute and nutrient transporters in placental barriers during hypoxia. Hypoxia results in reduced expression and functional activity of amino acid and glucose transporters in placental barriers. Hypoxia also alters the expression and functional activity of transporters in kidney, liver, intestines, and cancerous tissues. Hypoxia reduces the expression and functional activity of amino acid transporters in lungs and intestines. Although tissue hypoxia is a cause of choroid/retinal disorders such as age related macular degeneration and diabetic retinopathy, there is dearth of knowledge on the effect of hypoxia on expression and activity of solute and nutrient transporters in retina. Previous studies characterized the effect of hypoxia on expression of glutamate and glucose transporters in whole retina and retinal capillary endothelial cells. Some studies have shown up regulation of expression and functional activity of glucose transporter (GLUT1) in retinal capillary endothelial cells under hypoxia and speculated its involvement in the pathology of diabetic retinopathy. Monitoring of hypoxia related changes in the expression of transporters is helpful in elucidating the disease mechanism, while allowing targeted drug delivery to the affected tissue.

The present inventors have for the first time characterized the expression of 84 transporters in hypoxic and normoxic rat choroid-retina. Further, the functional activity of four solute carrier transporters (SLC), including peptide transporters (PEPT), amino acid transporters (ATB⁰⁺), organic cation transporters (OCT), and monocarboxylate transporters (MCT), that are useful for transporter guided drug delivery were compared between hypoxic and normoxic conditions. PEPT and ATB⁰⁺ were chosen for further study in functional characterization because these transporters have broad substrate specificity and high transport capacity. OCT and MCT were chosen because most of the ocular drugs are either cationic or anionic molecules. These ionic drug molecules may be transported across ocular barriers either through OCT or MCT transporters. Functional activity of PEPT, ATB⁰⁺, OCT, and MCT transporter was compared by measuring the transport of specific substrates across hypoxic and normoxic calf sclera-choroid-RPE (SCRPE) and cornea.

Materials and Methods:

Materials required for RNA isolation and q-RT-PCR were purchased from Qiagen (Qiagen, Valencia, Calif.). MPP⁺ iodide, α-methyl-DL-tryptophan, phenyl acetic acid, valacylovir, Gly-Sar, metformin, nicotinic acid sodium salt, nadolol and formic acid were purchased from Sigma-Aldrich (St. Louis, Mo.). H-Pro-Phe-OH was purchased from Bachem (Torrance, Calif.). HPLC grade acetonitrile and methanol were purchased from Fisher Scientific (Fair Lawn, N.J.). Ammonium formate was purchased from Fluka BioChemika (USA). All other chemicals and reagents used in this study were of analytical reagent grade.

Calf and Rat Ocular Tissues:

Animals used in this study were those that were sacrificed as part of other experiments approved by the Institutional Animal Care Committee of the Colorado State University (Fort Collins) and University of Colorado Anschutz Medical campus. Hypoxic and normoxic calf eyes were obtained from the Department of Physiology, School of Veterinary Medicine, Colorado State University (Fort Collins, Colo.). Briefly, 1 day old male Holstein calves (n=4) were kept in hypobaric hypoxic chambers (P_{B}=445 mm Hg) for 2 weeks. For control experiment, age matched calves (n=4) were kept at ambient altitude (P_{B}=650 mm Hg) and normoxia for two weeks. Hypoxic and normoxic rat eyes were obtained from the Department of Medicine, University of Colorado Anschutz Medical campus (Aurora, Colo.). Sprague Dawley rats (n=4) were maintained in hypobaric hypoxic chambers (P_{B}=380 mm Hg) for 6 weeks and age matched controls were kept at ambient pressure and normoxia.

RNA Extraction and Quality Control Analysis (Provide Catalog Numbers for Each and Every qPCR Reagent):

Isolation of RNA from rat ocular tissues was carried out using QIAzol and RNeasy mini kit as per manufacturer's protocol (Qiagen, Valencia, Calif.). Rat eyes were isolated immediately after euthanasia, snap frozen in liquid nitrogen and stored at −80° C. until further processing. Eyes were dissected in a frozen condition on an ice-cold ceramic tile placed on a dry ice isopentane bath. Whole choroid-retina was isolated and transferred into RNase free microcentrifuge tube containing 300 μl of RNAlater solution (Qiagen Inc.) and stored at −80° C. until further processing. At the time of RNA isolation, tissues were removed from RNAlater solution and transferred into a tube containing QIAzol regent (10 times the volume of tissue weight) and homogenized. The isolated total RNA was then further purified using RNeasy mini purification kit. On column DNase digestion was carried out during RNA purification to eliminate genomic DNA contamination using a DNA elimination kit (Qiagen, Valencia, Calif.). Quality control analysis of isolated RNA samples for quantity, purity, and integrity was analyzed using Agilent Bioanalyzer before proceeding to the next step.

First Strand cDNA Synthesis:

Synthesis of first strand cDNA from isolated RNA samples was carried out using SABiosciences's RT² First Strand Kit as per manufacturer's protocol (Qiagen, Valencia, Calif.). Briefly, all reagents were centrifuged for 15 seconds before use. Genomic DNA contamination from the RNA sample (2.5 μg RNA) was removed by heating the samples at 42° C. for 5 minutes genomic DNA elimination buffer. For first strand cDNA synthesis, 10 μl of reverse transcriptase cocktail mixture was incubated with 10 μl of RNA sample treated with genomic DNA elimination mixture. Subsequently, the mixture was incubated at 42° C. for 15 minutes and then heated at 95° C. for 5 minutes. Synthesized cDNAs were diluted (dilution factor?) with water (92 μl) and stored at −80° C. until further use.

qPCR:

qPCR was performed using 96-well rat drug transporter PCR assay plates and ABI 7900HT FAST block as per manufacturer's protocol (Qiagen, Valencia, Calif.). PCR reaction mixture was prepared by mixing 1350 μl of SABiosciences RT² qPCR master mix, 102 μl of cDNA synthesized and diluted in the above step, and 1248 μl of water. PCR reaction was run in a total incubation volume of 25 μl. The PCR was run at 95° C. for 10 minutes, followed by 40 cycles of 95° C. for 15 seconds each, and then 60° C. for 1 minute each, followed by final elongation at 72° C. for 15 minutes after the last PCR cycle.

Relative Gene Expression Analysis:

Relative gene expression analysis was performed by normalization of gene expression using five rat reference genes, including ribosomal protein P1 (RPLP1), hypoxanthine phosphoribosyltransferase 1(HPRT1), ribosomal protein L13A (RPL13A), lactate dehydrogenase-A (LDHA) and β-actin. The geometric mean of five rat reference genes was used as a normalization factor for relative quantification of each gene. The difference in Ct (ΔCt) for each gene in the plate was calculated as the difference between Ct values for the gene of interest and the geometric mean of Ct for reference genes. The fold change in relative gene expression between hypoxic and normoxic choroid-retina was calculated using web based PCR data analysis software (SABiosciences website-http://pcrdataanalysis.sabiosciences.com/per/arrayanalysis.php).

In Vitro Transport Across Calf Cornea and Sclera-Choroid-RPE:

In vitro transport studies across hypoxic and control calf cornea and sclera-choroid-RPE (SCRPE) were carried using cassette dosing approach. A cassette of drug transporter substrates, Gly-Sar (PEPT), valacylovir (ATB⁰⁺), MPP⁺ (OCT), and phenylacetic acid (MCT) at a concentration of 100 μM each in assay buffer was prepared. Briefly, the calf eyes were harvested immediately after euthanasia and transferred to the laboratory on ice. Upon arrival in the lab, the eyes were washed with assay buffer and cleaned from muscle and unwanted tissues. Anterior and posterior parts were separated by circumferential cut at the limbus. Vitreous was removed and the neural retina was separated from the choroid-RPE by filling the eye cup with assay buffer. The retina afloat in assay buffer was isolated and the eye cup was divided into two pieces (˜1.5×1.5 cm) of sclera-choroid-RPE. Isolated tissues were mounted on modified Ussing chambers (Navicyte, Sparks, Nev.) such that the episcleral side of SCRPE or epithelial side of cornea was facing the donor chamber and retinal side or endothelial side of the cornea was facing the receiver chamber. The chambers were filled with 1.5 ml of assay buffer at 37° C. with (donor side) or without (receiver side) the cocktail of drug transporter substrates. For the study of effect of transporter inhibitors, cocktail mixture (500 μM) of transporter inhibitors was added on both donor and acceptor sides. Summary of specific transporter substrates and inhibitors used for transport study are provided in the following Table:

TABLE
List of transporter, specific substrates and inhibitors
for particular transporter and inhibition mechanism.

| Trans- | Specific | Specific | |
|---|---|---|---|
| porter | Substrate | Inhibitor | Inhibition Mechanism |
| PEPT | Gly-Sar | H-Pro-Phe-OH | Competitive Inhibition |
| OCT | MPP+ | Metformin | Competitive Inhibition |
| ATB⁰⁺ | Valacylovir | α-Methyl | Specific Inhibition |
| | | Tryptophan | |
| MCT | Phenyl Acetic | Nicotinic acid | Competitive Inhibition |
| | Acid | | |

During the transport study, the bathing fluids were maintained at 37° C. using circulating warm water. The pH of the fluids was maintained at pH 7.4 using 95% air-5% CO₂ aeration. Samples were collected (200 μL) from receiver side every hour for 6 hours and the removed volume was replaced with fresh assay buffer pre-equilibrated to 37° C. Drug levels were analyzed using a LC-MS/MS assay. Permeation data were corrected for dilution of the receiver concentrations with sample volume replenishment.

LC-MS/MS Analysis:

Analyte concentrations in transport study samples were measured using LC-MS/MS method after 5-fold dilution with acetonitrile to reduce the salt concentrations. A cassette analysis method was developed for simultaneous analysis of Gly-Sar, valacylovir, and MPP Phenyl acetic acid was analyzed separately with a negative ionization method and a normal phase separation method. An API-3000 triple quadrupole mass spectrometry (Applied Biosystems, Foster City, Calif., USA) coupled with a PerkinElmer series-200 liquid chromatography (Perkin Elmer, Waltham, Mass., USA) system was used for analysis. Gly-Sar, valacylovir and MPP were separated on Supelco C-5 column (2.1×10 mm, 3 μm) using water containing 0.1% formic acid (A) and acetonitrile:methanol (50:50 v/v) containing 0.1% formic acid (B) as mobile phase. A linear gradient elution at a flow rate of 0.3 ml/min with a total run time of 9 min was employed. Phenyl acetic acid was separated in normal phase separation mode on Obelisc-N silica column (2.1×10 mm, 3 μM) using 5 mM ammonium formate at pH 3.5 (A) and acetonitrile (B) as mobile phase. A linear gradient mode at a flow rate of 0.3 ml with a total run time of 6 min was used. Gly-Sar, valacylovir, and MPP were analyzed in positive ionization mode with the following multiple reaction monitoring (MRM) transitions: 147→90 (Gly-Sar); 325→152 (valacylovir); and 170→128 (MPP⁺). Phenyl acetic acid was analyzed in negative ionization mode with the following multiple reaction monitoring (MRM) transitions: 135→91 (Phenyl acetic acid).

Data Analysis:

All values in this study are expressed as mean±S.D. Statistical comparisons between two groups were determined using independent sample Student's t-test. Differences were considered statistically significant at p<0.05.

### Results

Quality Control Analysis of RNA Extracted from Rat Choroid-Retina:

Quality control analysis of isolated RNA samples were conducted as per the minimum information for publication of quantitative real-time PCR experiments (MIQE) guidelines. Integrity and purity of isolated RNA samples were analyzed by Agilent Bioanalyzer. Only samples with RNA integrity number (RIN) above 7 and rRNA ratio (28s/18s) above 1.5 were used in the qRT-PCR analysis. RNA concentration, RIN, and rRNA ratio for samples used in the current study are summarized in the following Table:

TABLE
Summary of RNA quality control analysis. RNA concentrations,
RNA integrity number (RIN), rRNA ratios, positive PCR control
(PPC), and reverse transcription control (RTC) used during qRT-
PCR of transporter gene expression analysis for mRNA isolated
from hypoxic and normoxic rat choroid-retina (CR).

| | RNA | | rRNA | Positive | ΔCt |
|---|---|---|---|---|---|
| | concen- | | Ratio | PCR | (Avg Ct |
| | trations | RIN | (28 s/ | Control | RTC − Avg |
| Sample Name | (ng/μl) | number | 18 s) | (Ct PPC) | Ct PPC) |
| Hypoxic-CR1 | 603 | 9.0 | 1.6 | 18.1 ± 0.17 | 3.58 |
| Hypoxic-CR2 | 898 | 8.9 | 1.6 | 20.6 ± 0.12 | 3.87 |
| Hypoxic-CR3 | 820 | 8.6 | 1.8 | 19.0 ± 0.29 | 4.65 |
| Normoxic-CR1 | 637 | 9.0 | 1.7 | 20.1 ± 0.21 | 3.86 |
| Normoxic-CR2 | 523 | 9.1 | 1.7 | 19.1 ± 0.23 | 3.82 |
| Normoxic-CR3 | 565 | 9.3 | 1.7 | 18.1 ± 0.19 | 4.10 |

All samples used in the current study had RIN numbers above 8.6 and rRNA ratios above 1.6. Genomic DNA contamination in each RNA samples was analyzed by inclusion of the genomic DNA control well in the RT-PCR plate. In each sample tested, genomic DNA contamination was absent. Further, the effect of impurities present in the RNA samples on reverse transcription and PCR amplification reaction was monitored by inclusion of 3 wells for reverse transcription control (RTC) and 3 wells for positive PCR control (PPC) in the qRT-PCR reaction. As per MIQE guidelines, the average Ct for PPC should be 20±2 and should not vary by more than 2 cycles between PCR arrays being compared. For the current set of samples the average Ct of PPC ranged from 18.1 to 20.9, which were within the acceptable limit (20±2). As per MIQE guidelines, ΔCt values (ΔCt=Average Ct for RTC−Average Ct for PPC) should be less than 5 to confirm that the isolated RNA samples are free from impurities. In our study we observed that ΔCt values ranged from 3.6 to 4.6, which were below the limit of 5.

Transporters mRNA Expression in Rat Choroid-Retina:

A summary of the transporter expression patterns in normal rat choroid-retina is shown in the following Table:

TABLE
Summary of expression of 84 transporter genes in normoxic rat choroid-retina. The table
summarizes the gene accession ID, common gene symbols, gene name, mean Ct value obtained
from three assays, and the expression level. Gene expression level was assigned based on
mean Ct values obtained from qRT-PCR reactions. Ct values above 35 were considered as absent
(A); Ct values in the range, 30 to 35, were considered as very low expression (VL); Ct
values in the range, 25 to 30, were considered as low to medium expression (L to M); and
Ct values less than or equal to 25 were considered as high expression (H).

| Accession ID | Symbol | Gene Name | Ct | Expression Level |
|---|---|---|---|---|
| NM_178095 | Abca1 | Abca1 | 25.306 | L to M |
| NM_001106020 | Abca13 | — | 27.543 | L to M |
| NM_001031637 | Abca17 | — | 34.605 | VL |
| NM_024396 | Abca2 | Abc2 | 26.064 | L to M |
| XM_220219 | Abca3 | — | 26.076 | L to M |
| NM_001107721 | Abca4 | ABCR | 20.166 | H |
| XM_221101 | Abca9 | — | 26.740 | L to M |
| NM_031760 | Abcb11 | Bsep/Spgp | 35.000 | A |
| NM_012623 | Abcb1b | Abcb1/Mdr1/Pgy1 | 29.213 | L to M |
| NM_012690 | Abcb4 | Mdr2/Pgy3 | 28.583 | L to M |
| XM_234725 | Abcb5 | RGD1566342 | 35.000 | A |
| NM_080582 | Abcb6 | MGC93242 | 28.936 | L to M |
| NM_022281 | Abcc1 | Abcc1a/Avcc1a/Mrp/Mrp1 | 22.511 | H |
| NM_001108201 | Abcc10 | MRP7 | 30.151 | VL |
| NM_199377 | Abcc12 | MRP9 | 33.427 | VL |
| NM_012833 | Abcc2 | Cmoat/Mrp2 | 27.545 | L to M |
| NM_080581 | Abcc3 | Mlp2/Mrp3 | 29.486 | L to M |
| NM_133411 | Abcc4 | Mrp4 | 27.113 | L to M |
| NM_053924 | Abcc5 | Abcc5a/MGC156604/Mrp5 | 25.199 | L to M |
| NM_031013 | Abcc6 | Mrp6 | 32.995 | VL |
| NM_001108821 | Abcd1 | RGD1562128 | 27.467 | L to M |
| NM_012804 | Abcd3 | PMP70/Pxmp1 | 23.421 | H |
| NM_001013100 | Abcd4 | MGC105956/Pxmp1l | 27.968 | L to M |
| NM_001109883 | Abcf1 | Abc50 | 22.129 | H |
| NM_181381 | Abcg2 | BCRP1 | 26.968 | L to M |
| NM_130414 | Abcg8 | — | 36.144 | A |
| NM_012778 | Aqp1 | CHIP28 | 24.506 | H |
| NM_019157 | Aqp7 | — | 36.575 | A |
| NM_022960 | Aqp9 | MGC93419 | 32.229 | VL |
| NM_130823 | Atp6v0c | Atp6c/Atp61 | 20.538 | H |
| NM_052803 | Atp7a | Mnk | 25.274 | L to M |
| NM_012511 | Atp7b | Hts/PINA/Wd | 26.844 | L to M |
| NM_022715 | Mvp | Major vault protein | 27.037 | L to M |
| NM_017047 | Slc10a1 | Ntcp/Ntcp1/SBACT | 26.505 | L to M |
| NM_017222 | Slc10a2 | ISBAT | 33.598 | VL |
| NM_057121 | Slc15a1 | Pept1 | 29.454 | L to M |
| NM_031672 | Slc15a2 | MGC91625 | 26.724 | L to M |
| NM_012716 | Slc16a1 | MCT1/RATMCT1/RNMCT1 | 21.038 | H |
| NM_147216 | Slc16a2 | MCt8 | 25.785 | L to M |
| NM_030834 | Slc16a3 | MCt3 | 26.788 | L to M |
| NM_017299 | Slc19a1 | MGC93506/MTX1 | 24.709 | H |
| NM_001030024 | Slc19a2 | MGC124887 | 24.562 | H |
| NM_001108228 | Slc19a3 | ThTr-2/Thiamine transporter 2 | 29.822 | L to M |
| NM_012697 | Slc22a1 | MGC93570/OCt1/OrCt1/RoCt1 | 35.589 | A |
| NM_031584 | Slc22a2 | OCT2/OCT2r/rOCT2 | 33.178 | VL |
| NM_019230 | Slc22a3 | OCT3/EMT | 33.420 | VL |
| NM_017224 | Slc22a6 | MGC124962/Oat1/OrCtl1/Paht/Roat1 | 34.916 | A |
| NM_053537 | Slc22a7 | Oat2 | 32.139 | VL |
| NM_031332 | Slc22a8 | MGC93369/OCT3/Oat3/RoCt | 26.089 | L to M |
| NM_173302 | Slc22a9 | Oat5/Slc22a19 | 34.306 | VL |
| XM_342640 | Slc25a13 | RGD1565889 | 27.579 | L to M |
| NM_053863 | Slc28a1 | Cnt1 | 35.825 | A |
| NM_031664 | Slc28a2 | Cnt2 | 25.951 | L to M |
| NM_080908 | Slc28a3 | Cnt3 | 29.543 | L to M |
| NM_031684 | Slc29a1 | rENT1 | 22.930 | H |
| NM_031738 | Slc29a2 | rENT2 | 28.633 | L to M |
| NM_138827 | Slc2a1 | GLUTB/GTG1/Glut1/Gtg3/RATGTG1 | 24.761 | H |
| NM_012879 | Slc2a2 | GTT2/Glut2 | 30.276 | VL |
| NM_017102 | Slc2a3 | GLUT3 | 26.216 | L to M |
| NM_133600 | Slc31a1 | Ctr1/LRRGT00200 | 23.622 | H |
| NM_181090 | Slc38a2 | Ata2/Atrc2/Sat2/Snat2 | 24.334 | H |
| NM_138854 | Slc38a5 | SN2 | 29.522 | L to M |
| NM_017216 | Slc3a1 | D2/NAA-TR/Nbat/rBAT | 20.857 | H |
| NM_019283 | Slc3a2 | Mdu1 | 22.919 | H |
| NM_013033 | Slc5a1 | MGC93553/SGLT1 | 27.548 | L to M |
| NM_001106383 | Slc5a4a | Slc5a4 | 32.581 | VL |
| NM_001107673 | Slc7a11 | Cystine/glutamate transporter | 24.996 | L to M |
| NM_001107078 | Slc7a4 | CAT4 | 30.213 | VL |
| NM_017353 | Slc7a5 | E16/TA1 | 24.981 | L to M |
| NM_001107424 | Slc7a6 | LAT3 | 25.441 | L to M |
| NM_031341 | Slc7a7 | y + LAT1 | 27.216 | L to M |
| NM_053442 | Slc7a8 | Lat2/Lat4 | 23.010 | H |
| NM_053929 | Slc7a9 | ATB⁰⁺ | 30.503 | VL |
| NM_030838 | Slco1a5 | OATP-3/Oatp3/Slc21a7/Slco1a2 | 24.564 | H |
| NM_130736 | Slco1a6 | Oatp5/Slc21a13 | 35.963 | A |
| NM_031650 | Slco1b3 | OATP-4/Oatp4/Slc21a10/Slco1b2/rlst-1 | 35.327 | A |
| NM_022667 | Slco2a1 | Matr1/Slc21a2 | 26.616 | L to M |
| NM_080786 | Slco2b1 | Slc21a9/moat1 | 27.831 | L to M |
| NM_177481 | Slco3a1 | Slc21a11 | 25.933 | L to M |
| NM_133608 | Slco4a1 | OATP-E/Slc21a12 | 22.944 | H |
| NM_032055 | Tap1 | Abcb2/Cim/MGC124549 | 30.635 | VL |
| NM_032056 | Tap2 | Abcb3/Cim/MGC108646 | 26.189 | L to M |
| NM_031353 | Vdac1 | Voltage-dependent anion channel 1 | 20.313 | H |
| NM_031354 | Vdac2 | Voltage-dependent anion channel 2 | 21.156 | H |

H = high expression (Ct ≦25); L to M = low to medium expression (Ct = 25-30); VL = very low expression (Ct = 30-35); and A = absent (Ct ≧35).

Transporters with a Ct value above 35 or undetermined during RT-PCR were considered absent. Out of 84 transporters tested, 9 transporters were absent in rat choroid-retina. Transporters present in the choroid-retina were divided into three categories based on their Ct values. Transporters with Ct values between 30 and 35 were considered as very low expression, Ct values between 25 and 30 were considered as low to medium expression, and transporters with Ct values below 25 were considered as high expression. Out of 75 transporters, 14 transporters exhibited very low expression, 40 transporters showed low to medium expression and only 18 showed high expressions. Transporters which showed high expression in choroid-retina were glucose transporters, monocarboxylate transporters, nucleoside transporters, organic anion transporting polypeptides, voltage dependent ion channels, aquaporin 1 transporter, folate and thiamine transporters, and efflux transporters including MRP1, ABCR and Abc50.

Effect of Hypoxia on ATP-Binding Cassette Transporters mRNA Expression:

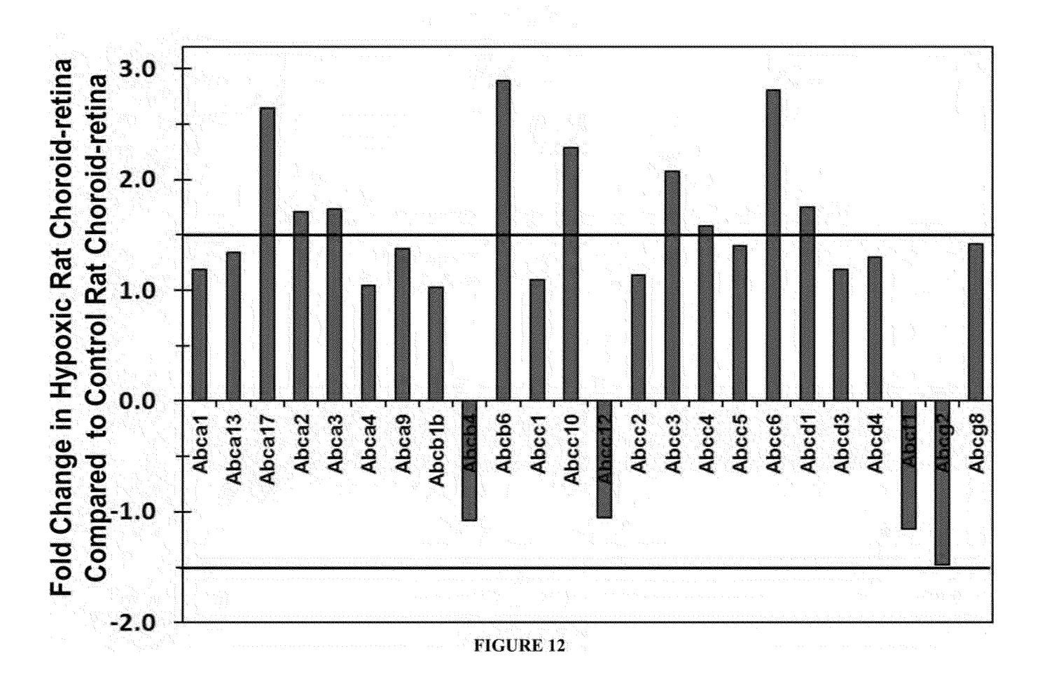

Relative gene expression analysis between hypoxic and control rat choroid-retina showed that out of 26 ABC transporters, 9 transporters were up regulated by 1.5-fold in hypoxic choroid-retina (FIG. 12). Transporters which were up regulated in hypoxia were MRP3, MRP4, MRP5, MRP (member 10), MDR6, Abca17, Abc2, Abc3, and RGD1562128.

Effect of Hypoxia on Solute Carrier Transporters mRNA Expression:

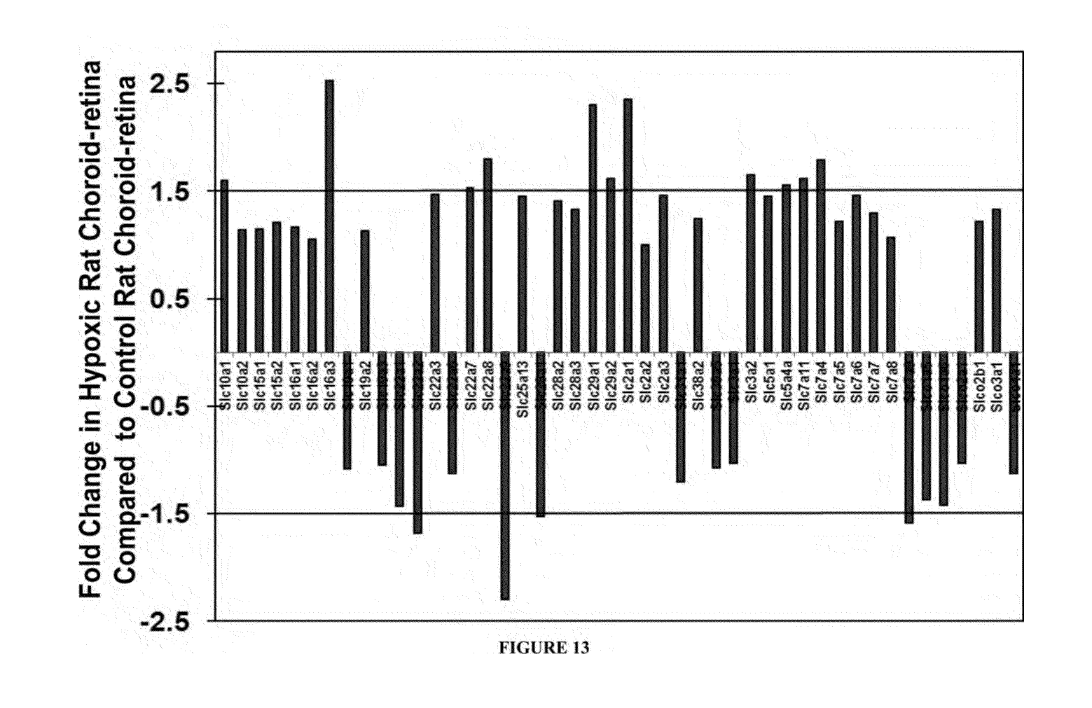

Relative gene expression analysis of solute carrier transporter (SLC) between hypoxic and control rat choroid-retina showed that out of 46 SLC transporters, 11 transporters were up regulated and 4 transporter were down regulated by ≧1.5-fold in hypoxic choroid-retina (FIG. 13). Transporters that were significantly up regulated in hypoxia are SBACT (sodium/bile acid co-transporter family; SLC10a1), MCT-3 and MCT-4 (monocarboxylate transporter-3; SLC16a3), OAT-2 (Organic anion transporter-2; SLC22a7), OAT-3 (Organic anion transporter-3; SLC22a8), ENT-1 (Equilibrative nucleoside transporters; SLC29a1), ENT-2 (Equilibrative nucleoside transporters; SLC29a2), GLUT-1 (Facilitated glucose transporters; SLC2a1), MDU-1 (activators of dibasic and neutral amino acid transporter; SLC3a2), SGLT2 (Low affinity sodium-glucose co-transporter; SLC5a4)), SLC7a11 (cationic amino acid transporter, y+ system; Cystine/glutamate transporter), SLC7a4 (cationic amino acid transporter, y+ system; CAT4). Transporters that were down regulated in hypoxic choroid-retina are OCT-2 (organic cation transporter 2; SLC22a2), OAT-5 (Organic anion transporter-5; SLC22a9), CNT-1 (sodium coupled concentrative nucleoside transporter; SLC28a1), and ATB⁰⁺ (B (0,+)-type, amino acid transporter; SLC7a9)

Effect of Hypoxia on Miscellaneous Transporters mRNA Expression:

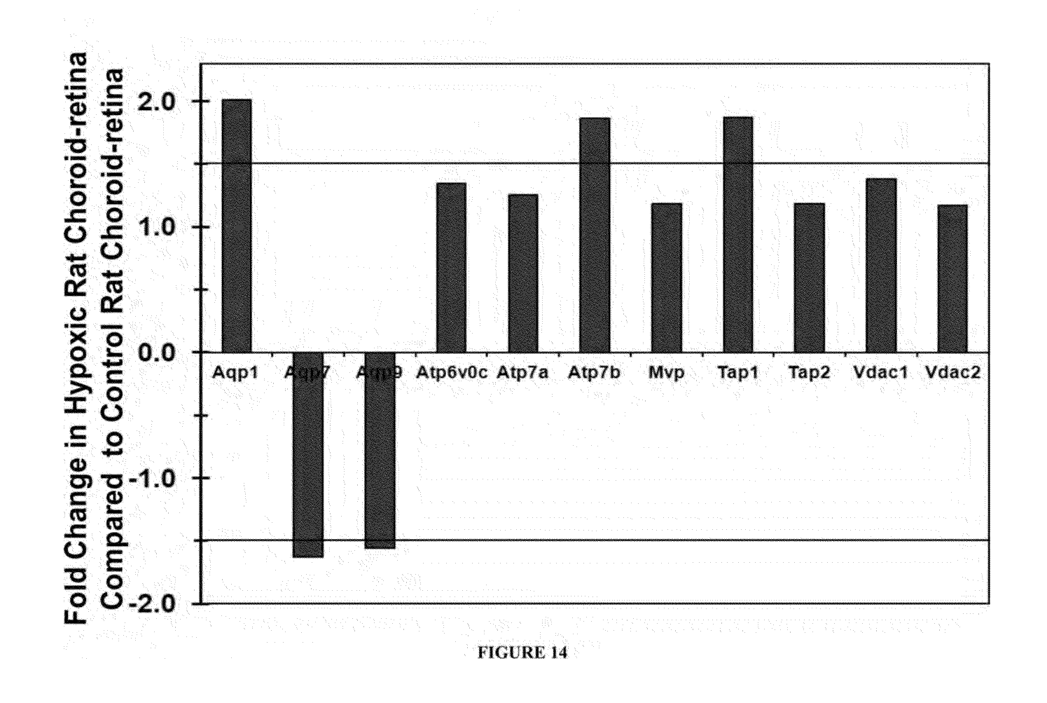

Relative gene expression analysis of miscellaneous transporters including aquaporin (Aqp), ATPase, voltage dependent ion channel, and TAP transporters between hypoxic and control rat choroid-retina are shown in FIG. 14. Aqp-1 was up regulated and Aqp-9 and Aqp-7 were down regulated in hypoxic choroid-retina. Further, ATPase-7b and TAP-1 were also up regulated by 1.5-fold in hypoxic choroid-retina.

Effect of Hypoxia on Transport of Transporter Substrate Cassette Across Calf SCRPE:

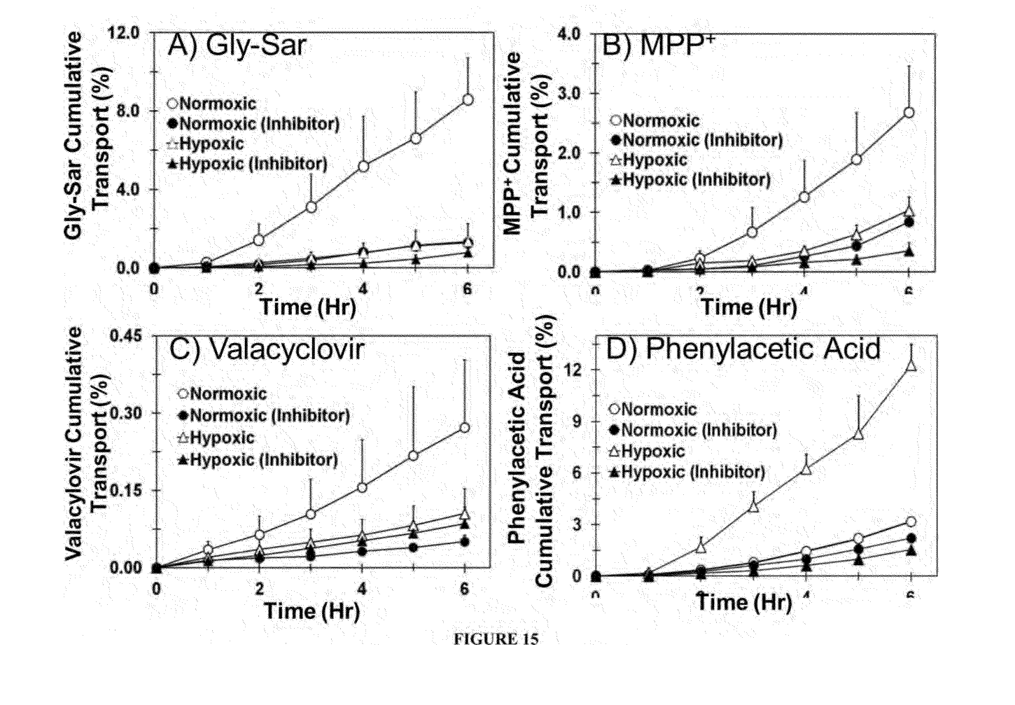

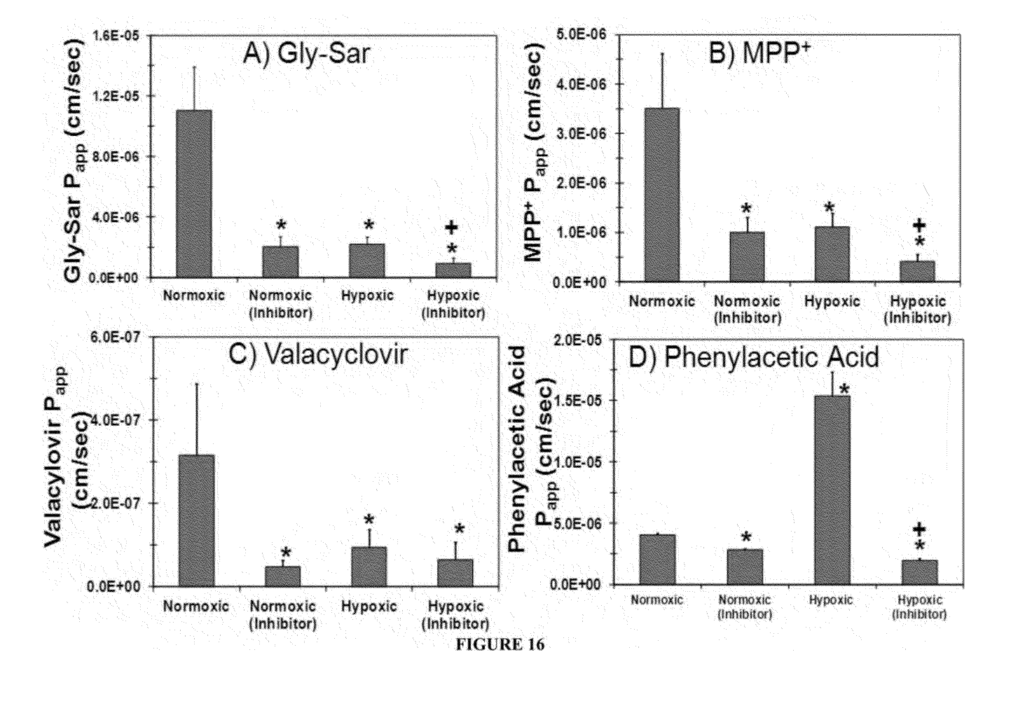

Transport of transporter substrate cassette across normoxic and hypoxic calf SCRPE was carried out to evaluate the effect of hypoxia on the functional activity of PEPT, ATB⁰⁺, OCT and MCT transporters in SCRPE. Transport studies were also conducted in the presence of transporter specific inhibitors to determine whether the transport is mediated by transporters. As shown in FIGS. 15 and 16, transport of Gly-Sar (PEPT substrate), valacylovir (ATB⁰⁺ substrate), and MPP⁺ (OCT substrate) was significantly decreased in hypoxic calf SCRPE when compared to age matched normoxic calf SCRPE. However, the cumulative % transport and apparent permeability constant (Papp) of phenyl acetic acid (MCT substrate) was increased by several fold in hypoxic condition (FIGS. 15D and 16D). Interestingly, the directionality of phenyl acetic transport is opposite in human SCRPE tissue (FIG. 7D). Moreover, FIG. 13 clearly shows that MCT3 and other OAT transporters are upregulated in hypoxic conditions. Thus, MCT mediated inward delivery is feasible in disease conditions. Transport of all four transporter substrates was significantly inhibited in the presence of transporter specific inhibitors in both normoxic and hypoxic conditions (FIGS. 15 and 16).

Effect of Hypoxia on Transport of Transporter Substrate Cassette Across Calf Cornea:

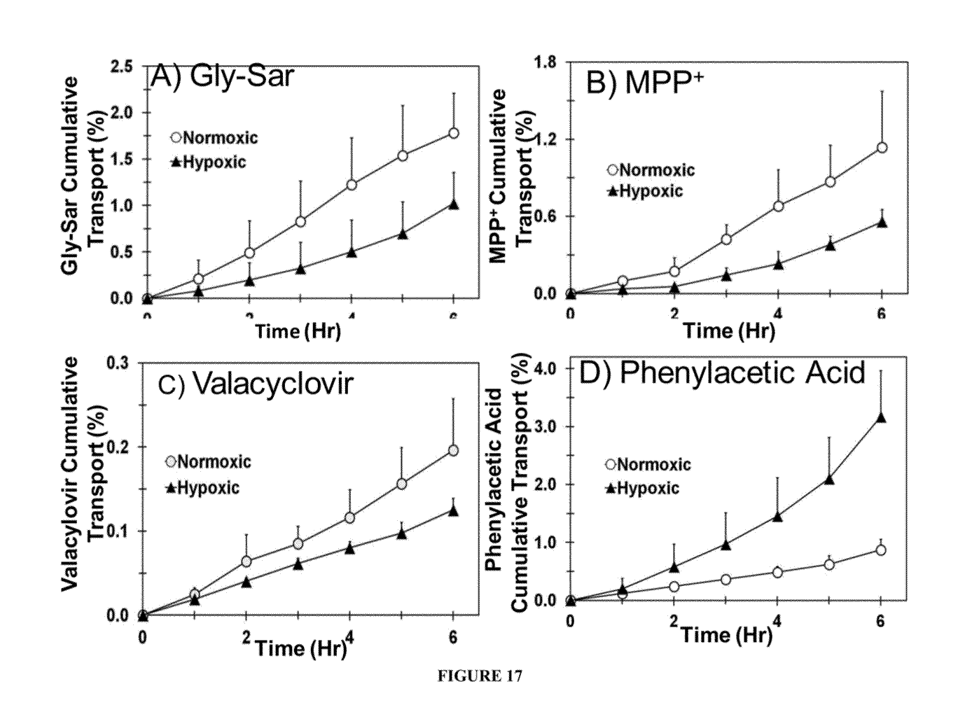

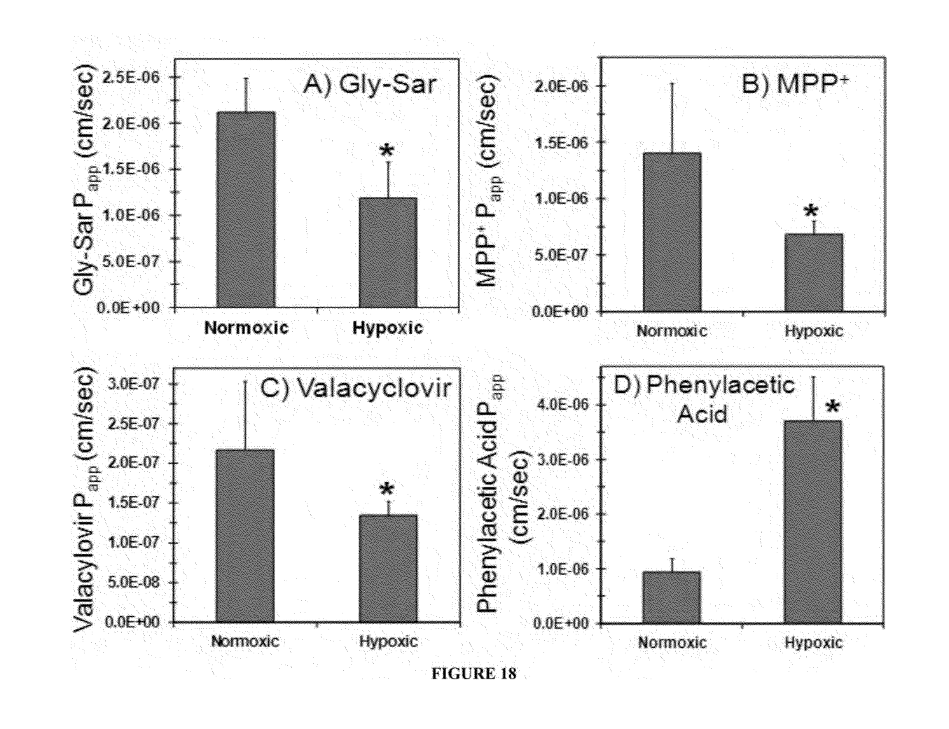

Transport of transporters substrate cassette across normoxic and hypoxic calf cornea was carried out to evaluate the effect of hypoxia on functional activity of PEPT, ATB⁰⁺ OCT and MCT in cornea. Similar to SCRPE, the functional activity of PEPT, ATB⁰⁺ and OCT transporters was significantly reduced in hypoxic cornea when compared to normoxic cornea. In the case of MCT, functional activity of MCT was significantly increased in hypoxic cornea (FIGS. 17 and 18).

### Discussion

The present inventors characterized the expression of 84 transporters in rat choroid-retina under normoxic and hypoxic conditions using RT² Profiler PCR array. Out of the 84 transporters tested, 9 transporters were absent in normal rat choroid-retina and only 18 showed abundant expression. Induction of hypoxia resulted in significant changes in the expression of transporters; out of 75 transporters present, 23 transporters were up regulated and 6 transporters were down regulated by greater than 1.5-fold when compared to age-matched normoxic controls. Both mRNA expression and functional activity of OCT and ATB⁰⁺ were down regulated in hypoxia. For PEPT, although functional activity was significantly down regulated in hypoxic SCRPE and cornea, mRNA analysis showed no change in the expression of PEPT under hypoxia. For MCT, gene expression and functional activity was up regulated in hypoxia.

Due to the highly dynamic nature of mRNA transcription and potential of variability depending on sample handling and processing, quality control analysis is of utmost importance to get the reproducible and reliable results during RT² Profiler PCR array analysis. Therefore, qRT-PCR experiments were conducted per MIQE guidelines to avoid assay-to-assay variability and to obtain reproducible and reliable results. As shown in Table above, quality control analysis of RNA samples passed all quality control tests with RIN above 7 and rRNA ratio (28s/18s) above 1.5. Further, the samples were free from genomic DNA contamination.

The present inventors characterization of the expression of 84 transporter genes in rat choroid-retina showed that out of 84 transporters, 9 transporters were absent and only 18 transporters showed abundant expression. Eighteen transporters, which showed abundant expression in rat choroid-retina were, voltage dependent anion channels (Vdac), OATP-E, OATP-1, LAT-2, LAT-1 (Slc3a2), B(0,+) types, amino acid transport protein (rBAT), amino acid transporter A2 (Ata2), copper transporter1 (Ctrl), glucose transporter 1 (Glut1), equilibrative nucleoside transporter 1 (ENT1), thiamine transporter (Thtr1), folate transporter (FLOT 1), monocarboxylate transporter 1 (MCT1), Atp6v0c, Aquaporin 1, ATP-binding cassette 50, ABCD3, MRP1, and ABCA4 (ABCR). ABCA4 is retina specific ABC transporter located in the outer segment of photoreceptor cells and is associated with autosomal retinal degenerative disorders. Most of the transporters that showed abundant expression in choroid-retina are nutrient transporters. Retina is a highly metabolically active tissue and needs a large amount of nutrient supply to maintain its metabolic needs. Amino acid transporters such as LAT, rBAT, and Ata2 showed abundant expression in the retina because retina needs a large amount of amino acids for synthesis of various neurotransmitters. Previous reports showed abundant expression of OATP-E and OATP-1 in rat ocular tissues, most specifically in retinal pigmented epithelium and retina and are involved in the transport of thyroid hormones and organic anions. Others have characterized the mRNA expression of drug transporters in human ocular tissues, but their study was limited to 21 transporters, including 5 ABC and 16 SLC transporters. However, in the present disclosure the expression of 84 transporters in choroid-retina were characterized.

Hypoxia results in significant alterations in the expression of transporter genes in rat choroid-retina, with ≧1.5 fold up-regulation of 23 transporters and ≧1.5 fold down regulation of 6 transporters. In the ABC transporter family, 9 transporters including MRP3, MRP4, MRP5, MRP (member 10), MDR6, Abca17, Abc2, Abc3, and RGD1562128 were up regulated in hypoxia (FIG. 12). Although it is not clear whether a hypoxia responsive element is present in the promoter region of ABC transporters, few studies have shown the up regulation of MRP and MDR transporters during hypoxia. ABCG2 or BCRP1 transporter was significantly down regulated in hypoxic choroid-retina. A previous study showed that the ABCG2 is up-regulated in hypoxic stem cells and acts as a cell survival factor by reducing cellular accumulation heme or porphyrin. A recent study showed the accumulation of porphyrin and heme in Bruch's membrane with age, implicating a role in AMD. Accumulation of porphyrin and heme in Bruch's membrane might be due to the down regulation of ABCG2 activity in choroid-retina as a result of hypoxia.

In the SLC transporter family, out of 46 SLC transporters, 11 transporters were up regulated and 4 transporters were down regulated by at least 1.5-fold in hypoxic choroid-retina (FIG. 13). Transporters that showed greater than 2-fold up regulation include MCT-3, GLUT-1, and ENT-1. MCT transporters mediate the diffusion of lactic acid and several other monocarboxylate compounds across plasma membrane. MCT-3 expression is largely restricted to the retinal pigmented epithelium in the eye and involved in the export of lactic acid produced by the retina to blood. Hypoxia stimulates the expression of various glycolytic enzymes including GLUT1 by transcriptional mechanisms involving hypoxia inducible factor. Increased GLUT1 levels in hypoxic retina stimulate lactate production. Some studies showed that the retinal lactate levels were 1.7-fold higher in hypoxic rat retina than age matched control rat retina. As MCT-3 is the predominant transporter involved in lactic acid export from the retina to choroid, MCT-3 expression is also increased during hypoxia. Others showed that only MCT3/4 but not MCT-1 is up regulated by hypoxia in HeLa and COS cells. The present inventors have also observed that only MCT-3 and not MCT-1 was up regulated in hypoxic choroid-retina. Previous literature reports reported down regulation of ENT transporters in hypoxia. However, the present inventors have observed that the expression of both ENT1 and ENT2 were up regulated in hypoxic choroid-retina.

Expression of GLUT1 as well as low affinity glucose transporter (Slc5a4a) was up regulated by 1.6-fold in hypoxic choroid-retina. Stimulation of expression of Slc5a4a in hypoxic conditions might be regulated by the transcriptional mechanisms involving hypoxia inducible factor similar to GLUT1; however, very little information is available on Slc5a4a. Other transporters up regulated in hypoxic choroid-retina were activators of dibasic and neutral amino acid transport (SLC3a2), Cystine/glutamate transporter (SLC7a11), and CAT4. Cystine/glutamate transporter provides intracellular cystine for the production of glutathione, a major cellular antioxidant. Induction of hypoxia results in the development of hypoxia related oxidative stress in choroid-retina. Increased expression of cystine/glutamate transporter in hypoxic choroid-retina provides protection from the oxidative stress. Over expression of cystine/glutamate transporter in hypoxic conditions increases the supply of intracellular cysteine for production of glutathione in neuronal cells, thereby protecting them from oxidative stress. Cationic amino acid transporters (CAT) are involved in the transport of arginine, which is a main precursor for nitric oxide synthesis. Hypoxia induces the synthesis of nitric oxide, depending on the supply of precursor L-arginine. L-arginine is a cationic amino acid and its intracellular transport is mediated by CAT. Increased nitric oxide production in hypoxic conditions up regulates CAT mRNA expression as a secondary mechanism to increase the supply of L-arginine.

SLC transporters down regulated in hypoxic choroid-retina include OCT-2, OAT5, CNT1, and ATB⁰⁺ (FIG. 13). Although no direct reports are available on the effect of hypoxia on OCT-1 and OCT-2 expression, literature reports suggest that hypoxia results in down regulation of expression of OCTN-2 in placenta and BeWo cells. OAT-5 expression in kidney was shown to be down regulated during ischemia. ATB⁰⁺ showed 1.6-fold down regulation during hypoxia which is consistent with previous literature reports, which showed down regulation of expression and activity of ATB⁰⁺ transporter during hypoxic and ischemic conditions.

Out of 11 miscellaneous transporters, 3 transporters including AQP-1, TAP-1 and ATP7b were up regulated and AQP-9 was down regulated by at least 1.5 fold (FIG. 14). In the aquaporin transporter family, AQP-1 was up regulated and AQP-9 was down regulated during hypoxia. AQP-1 is a water channel protein which shows abundant expression in red blood cells and tissues with rapid O₂ transport. It is known to be up regulated during hypoxia through hypoxia inducible factor and it is associated with inflammatory edema and tumor growth. Others showed that AQP1 is required for hypoxia induced angiogenesis of human retinal vascular endothelial cells and inhibition of AQP1 inhibits angiogenesis. Still others showed the expression of AQP9 in retinal pigment epithelial cells (ARPE-19) and its involvement in the transport of various uncharged molecules such lactate, glycerol, purines, pyrimidines, urea, and mannitol. Hypoxia results in significant down regulation of AQP-9 in rat astrocytes, and subsequent reoxygenation results in restoration of expression of AQP-9 to the basal level. Other transporters that were up regulated by hypoxia include TAP1 and ATP7b. ATP7a and ATP7b are copper transporting ATPases that transport copper across cellular membranes and hypoxia is known to up regulate the activity of ATP7a and ATP7b.

Effect of hypoxia on the functional activity of four solute carrier transporters including PEPT, ATB⁰⁺ OCT, and MCT was evaluated using hypoxic and normoxic calf ocular tissues. Although the mRNA expression is responsible for protein expression and activity, there are many instances in which mRNA levels show poor correlation with protein levels. This is because many complicated post-transcriptional mechanisms are involved in turning mRNA into proteins; and second, different proteins have different biological half-lives in vivo. Evaluation of functional activity of selected proteins gives a more realistic picture of disease status and helps to rule out uncertainty. Due to the small dimensions of rat eyes, in vitro transport studies across isolated rat ocular tissues are difficult to perform. Therefore, for in vitro transport study, the ocular tissues obtained from hypoxic and normoxic calves were used. Gene expression analysis was not performed in calf ocular tissues because of the difficulty in obtaining the PCR probes for bovine transporters; however, for rat transporters, RT-PCR profiler arrays were readily available. A cassette dosing approach was used to increase the throughput.

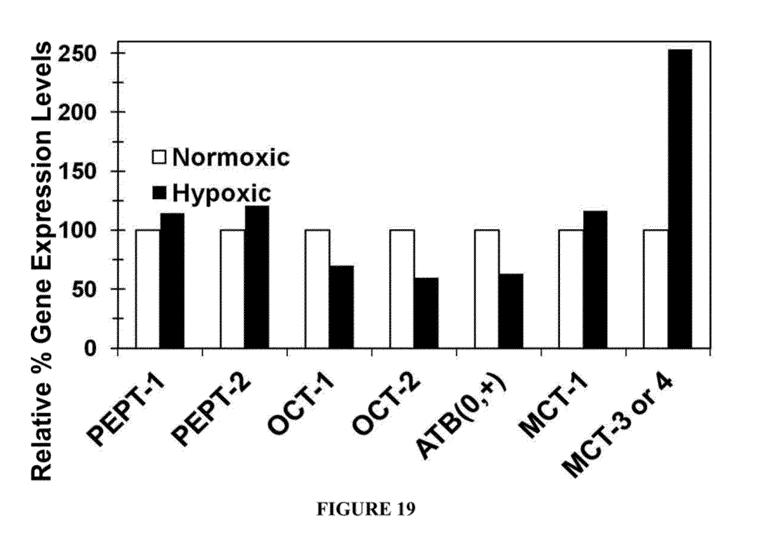

Induction of hypoxia resulted in a significant reduction in functional activity of PEPT, ATB⁰⁺, and OCT and an increase in the activity of MCT transporters in hypoxic calf SCRPE and cornea, when compared with normoxic controls (FIGS. 16 and 18). Functional activity data observed for ATB⁰⁺, OCT, and MCT in hypoxic calf SCRPE and cornea corroborate with gene expression analysis data observed in hypoxic rat choroid-retina. As shown in FIG. 19, hypoxia resulted in a reduction of mRNA expression of OCT-1 and OCT-2 by 30 and 41% in rat choroid-retina, respectively. The cumulative % transport of MPP⁺ (OCT substrate) across hypoxic SCRPE and cornea was decreased by 61 and 49%, respectively (FIGS. 14 and 16). MPP⁺ used as a substrate for OCT transporter has broad specificity and interacts with both OCT as well as OCTN transporters. Hypoxia is known to down regulate both OCT as well as OCTN transporter activity. Hypoxia resulted in a decrease in cumulative % transport of valacylovir (ATB⁰⁺ substrate) across hypoxic calf SCRPE and cornea by 61 and 36%, respectively (FIGS. 14 and 16). Further, a decrease in mRNA expression of ATB⁰⁺ by 37% was observed in rat choroid-retina (FIG. 18).

In case of PEPT transporters, functional assay showed significant reduction in the transport of Gly-Sar (PEPT substrate) across hypoxic calf SCRPE and cornea (FIGS. 16 and 18). Gene expression analysis of PEPT transporters in hypoxic rat choroid-retina showed that there is no effect of hypoxia on PEPT expression (FIG. 19). Disagreement between these data sets might be due to hypoxia not altering the PEPT gene expression but affecting the protein stability and functional activity of PEPT transporters.

Hypoxia resulted in an increase in both expression as well as functional activity of MCT transporters in ocular tissues. In hypoxic choroid-retina, expression of MCT-3 and MCT-1 increased by 253 and 116%, respectively. In vitro transport studies across calf SCRPE and cornea also showed several fold increase in cumulative % transport and apparent permeability across hypoxic tissue than normoxic controls (FIGS. 15 to 17). The effect of hypoxia on the functional activity of MCT transporters was more prominent in SCRPE than cornea. Interestingly, there is a switch in the directionality of phenyl acetic acid transport across SCRPE in hypoxic calf (sclera to retina) and human (retina to sclera) species. One can potentially utilize these findings to enhance the delivery of ocular drugs or drug-ion pairs that are substrates for MCT or OAT transporters in diseases associated with hypoxia.

### CONCLUSIONS

In summary, this Example shows the mRNA expression and effect of hypoxia on the expression for 84 transporters in rat ocular tissues and functional activity of 4 SLC transporters in calf ocular tissues. Out of 84 transporters tested, 9 transporters were absent and only 18 transporters showed abundant expression in rat choroid-retina. Hypoxia results in significant alteration (≧50% up regulation or down regulation) in the expression of drug transporters in rat choroid-retina. Nine out of 29 ATP binding cassette (ABC) families of efflux transporters including MRP3, MRP4, MRP5, MRP6, MRP7, Abca17, Abc2, Abc3, and RGD1562128 were up regulated. For solute carrier family transporters, 11 transporters including SLC10a1, SLC16a3, SLC22a7, SLC22a8, SLC29a1, SLC29a2, SLC2a1, SLC3a2, SLC5a4, SLC7a11, and SLC7a4 were up regulated, while 4 transporters including SLC22a2, SLC22a9, SLC28a1, and SLC 7a9 were down regulated in hypoxic rat choroid-retina. Functional activity assays in hypoxic calf cornea and SCRPE showed down regulation of PEPT, ATB⁰⁺, and OCT activity, whereas upregulation was observed for MCT activity.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A composition comprising an ion-drug complex for treating an ocular clinical condition in a subject, wherein said ion-drug complex comprises an ionic complex of an ocular drug for treating the ocular clinical condition and a counterion that increases active transport of said ocular drug across an ocular cell of the subject by a membrane transporter.

2. The composition of claim 1, wherein the membrane transporter comprises an organic cation transporter (OCT), a monocarboxylate transporter (MCT), an amino acid transporter (ATB), a peptide transporter (PEPT), or a combination thereof.

3. The composition of claim 1, wherein the membrane transporter comprises OCT-1, OCT-2, MCT-1, MCT-3, PEPT-1, PEPT-2, ATB⁰⁺, or a combination thereof.

4. The composition of claim 1, wherein said ocular drug comprises a fluoroquinolone, an analog of prostaglandin, a beta-blocker, a non-steroidal anti-inflammatory compound, a corticosteroid, an anti-angiogenic compound, a neuroprotective compound, a cell survival compound, an anti-proliferative compound, an apoptotic compound, or a combination thereof.

5. The composition of claim 1, wherein said ocular drug comprises gatifloxacin, besifloxacin, celecoxib, diclofenac, ketorolac, nepafenac, bromfenac, timolol, brimonidine, betaxolol, or a combination thereof.

6. A method for treating an ocular clinical condition in a subject comprising administering to a subject in need of such a treatment a therapeutically effective amount of a composition of claim 1.

7. The method of claim 6, wherein the ocular clinical condition comprises inflammation, microbial infection, allergy, dry eye, glaucoma, surgery, diabetic retinopathy, retinal degeneration, macular degeneration, vascular occlusions, optic neuropathy, cataracts, posterior capsular opacification, corneal angiogenesis, other neovascular diseases, thyroid eye disease, retinoblastoma, uveal melanoma, endophthalmitis, or a combination thereof.

8. The method of claim 6, wherein the composition of claim 1 is actively transported by a membrane transporter.

9. The method of claim 8, wherein the membrane transporter comprises OCT-1, OCT-2, MCT-1, MCT-3, PEPT-1, PEPT-2, ATB⁰⁺, or a combination thereof.

10. The method of claim 6, wherein the composition of claim 1 comprises a therapeutically effective amount of the ocular drug selected from the group consisting of a fluoroquinolone, an analog of prostaglandin, a beta-blocker, a non-steroidal anti-inflammatory compound, a corticosteroid, an anti-angiogenic compound, a neuroprotective compound, a cell survival compound, an anti-proliferative compound, an apoptotic compound, and a combination thereof.

11. The method of claim 6, wherein the ocular drug comprises gatifloxacin, besifloxacin, celecoxib, diclofenac, ketorolac, nepafenac, bromfenac, timolol, brimonidine, betaxolol, pazopanib or a combination thereof.

12. A method for increasing the delivery of a compound to a desired ocular cell, said method comprising administering the compound as an ion-compound complex, wherein the ion-drug complex comprises an ionic complex of the compound and a counterion that increases active transport of the compound to the desired ocular cell.

13. The method of claim 12, wherein the compound is an ocular drug.

14. The method of claim 13, wherein the ocular drug comprises a fluoroquinolone, an analog of prostaglandin, a beta-blocker, a non-steroidal anti-inflammatory compound, a corticosteroid, an anti-angiogenic compound, a neuroprotective compound, a cell survival compound, an anti-proliferative compound, an apoptotic compound, a tyrosine kinase inhibitor or a combination thereof.

15. The method of claim 14, wherein the ocular drug comprises gatifloxacin, besifloxacin, celecoxib, diclofenac, ketorolac, nepafenac, bromfenac, timolol, brimonidine, betaxolol, or a combination thereof.

16. The method of claim 12, wherein the ion-compound complex is actively transported by a membrane transporter.

17. The method of claim 16, wherein the membrane transporter comprises an organic cation transporter (OCT), a monocarboxylate transporter (MCT), an amino acid transporter (ATB), a peptide transporter (PEPT), or a combination thereof.

18. The method of claim 16, wherein the membrane transporter comprises OCT-1, OCT-2, MCT-1, MCT-3, PEPT-1, PEPT-2, ATB⁰⁺, or a combination thereof.
